SEQUENCE LISTING (153 sequences)

SEQ ID NO: 1 - protein, 15 aa, synthetic construct
CTASGVNIKDTYMYW

SEQ ID NO: 2 - protein, 13 aa, synthetic construct
GRIDPANGYTRYD

SEQ ID NO: 3 - protein, 14 aa, synthetic construct
CAVNDGYFEYFDVW

SEQ ID NO: 4 - protein, 18 aa, synthetic construct
CKSSQSLLDSDGKPYLNW

SEQ ID NO: 5 - protein, 7 aa, synthetic construct
LVSKLDS

SEQ ID NO: 6 - protein, 11 aa, synthetic construct
CWQDTHFPRTF

SEQ ID NO: 7 - protein, 15 aa, synthetic construct
CTDSGFNIKDTYIHW

SEQ ID NO: 8 - protein, 13 aa, synthetic construct
GRIDPANGFTRYD

SEQ ID NO: 9 - protein, 15 aa, synthetic construct
CAREDGYPYYTLDCW

SEQ ID NO: 10 - protein, 18 aa, synthetic construct
CKSSQSLLDVDGKTYLNW

SEQ ID NO: 11 - protein, 7 aa, synthetic construct
LVSKLDS

SEQ ID NO: 12 - protein, 11 aa, synthetic construct
CWQGTHFPRTF

SEQ ID NO: 13 - protein, 119 aa, synthetic construct
EVQLQQSGAELVKPGASVKLSCTASGVNIKDTYMYWVKQRPEQGLEWIGRIDPANGYTRYDPKFQDKATVTADTSSNTAHLQLSSLTSEDTAVYYCAVNDGYFEYFDVWGAGTTVTVSS

SEQ ID NO: 14 - protein, 112 aa, synthetic construct
DVVMTQAPLTLSVTIGQPASISCKSSQSLLDSDGKPYLNWLLQRPGQSPKRLIYLVSKLDSGVPDRFTGSGSGTDFTLKISRVEAEDLGVYYCWQDTHFPRTFGGGTKLEIK

SEQ ID NO: 15 - protein, 120 aa, synthetic construct
EVQLQQSGAELVKPGASVKLSCTDSGFNIKDTYIHWVTQRPQQGLEWIGRIDPANGFTRYDPKFQGKATITSDTSSNTAYLQLSSLTSEDTAVYYCAREDGYPYYTLDCWGQGTSVTVSS

SEQ ID NO: 16 - protein, 112 aa, synthetic construct
DVVMTQSPLTLSLTIGQPASISCKSSQSLLDVDGKTYLNWFLQRPGQSPKRLIYLVSKLDSGVPDRFTGSGSGTDFTLKISRVEAEDLGVYYCWQGTHFPRTFGGGTKLEIK

SEQ ID NO: 17 - protein, 119 aa, synthetic construct
QVQLVQSGAEVKKPGASVKVSCKASGVNIKDTYMYWVRQAPGQGLEWIGRIDPANGYTRYAQKFQGRVTMTADTSTSTVYMELSSLRSEDTAVYYCAVNDGYFEYFDVWGQGTTVTVSS

SEQ ID NO: 18 - protein, 112 aa, synthetic construct
DVVMTQSPLSLPVTLGQPASISCRSSQSLLDSDGKPYLNWLQQRPGQSPRRLIYLVSKRDSGVPDRFSGSGSGTDFTLKISRVEAEGVGVYYCWQDTHFPRTFGQGTKLEIK

SEQ ID NO: 19 - protein, 119 aa, synthetic construct
QVQLVQSGAEVKKPGASVKVSCKASGVNIKDTYMYWVRQAPGQGLEWIGRIDPANGYTRYAQKFQGRATVTADTSTSTVYLELSSLRSEDTAVYYCAVNDGYFEYFDVWGQGTTVTVSS

SEQ ID NO: 20 - protein, 112 aa, synthetic construct; identical to SEQ ID NO: 18

SEQ ID NO: 21 - protein, 119 aa, synthetic construct
QVQLVQSGAEVKKPGSSVKVSCKASGVNIKDTYMYWVRQAPGQGLEWIGRIDPANGYARYAQKFQGRATITADESTSTAYLELSSLRSEDTAVYYCAVNDGYFEYFDVWGQGTTVTVSS

SEQ ID NO: 22 - protein, 112 aa, synthetic construct; identical to SEQ ID NO: 18

SEQ ID NO: 23 - protein, 119 aa, synthetic construct
QVQLVQSGAEVKKPGSSVKVSCKASGVNIKDTYMYWVRQAPGQGLEWIGRIDPANGYARYAQKFQGRATVTADKSTSTAYLELSSLRSEDTAVYYCAVNDGYFEYFDVWGQGTTVTVSS

SEQ ID NO: 24 - protein, 112 aa, synthetic construct; identical to SEQ ID NO: 18

SEQ ID NO: 25 - protein, 119 aa, synthetic construct
EVQLLESGGGLVQPGGSLRLSCAASGVNIKDTYMYWVRQAPGKGLEWIGRIDPANGYTRYADSVKGRFTISADNSKNTLYLQMNSLRAEDTAVYYCAVNDGYFEYFDVWGQGTTVTVSS

SEQ ID NO: 26 - protein, 112 aa, synthetic construct; identical to SEQ ID NO: 18

SEQ ID NO: 27 - protein, 119 aa, synthetic construct
QVQLQESGPGLVKPSGTLSLTCAASGVNIKDTYMYWVRQPPGKGLEWIGRIDPANGYTRYNPSFKSRATVSADKSKNQASLKLSSLTAADTAVYYCAVNDGYFEYFDVWGQGTTVTVSS

SEQ ID NO: 28 - protein, 112 aa, synthetic construct; identical to SEQ ID NO: 18

SEQ ID NO: 29 - protein, 119 aa, synthetic construct
EVQLVQSGAEVKKPGESLKISCKGSGVNIKDTYMYWVRQMPGKGLEWIGRIDPANGYTRYSPSFQGQATVSADKSISTAYLQLSSLKASDTAMYYCAVNDGYFEYFDVWGQGTTVTVSS

SEQ ID NO: 30 - protein, 112 aa, synthetic construct; identical to SEQ ID NO: 18

SEQ ID NO: 31 - protein, 119 aa, synthetic construct; identical to SEQ ID NO: 17

SEQ ID NO: 32 - protein, 112 aa, synthetic construct
EIVLTQSPGTLSLSPGERATLSCRSSQSLLDSDGKPYLNWLQQKPGQAPRRLIYLVSKLDSGIPDRFSGSGSGTDFTLTISRLEPEDLAVYYCWQDTHFPRTFGQGTKLEIK

SEQ ID NO: 33 - protein, 119 aa, synthetic construct; identical to SEQ ID NO: 19

SEQ ID NO: 34 - protein, 112 aa, synthetic construct; identical to SEQ ID NO: 32

SEQ ID NO: 35 - protein, 119 aa, synthetic construct; identical to SEQ ID NO: 21

SEQ ID NO: 36 - protein, 112 aa, synthetic construct; identical to SEQ ID NO: 32

SEQ ID NO: 37 - protein, 119 aa, synthetic construct; identical to SEQ ID NO: 23

SEQ ID NO: 38 - protein, 112 aa, synthetic construct; identical to SEQ ID NO: 32

SEQ ID NO: 39 - protein, 119 aa, synthetic construct; identical to SEQ ID NO: 25

SEQ ID NO: 40 - protein, 112 aa, synthetic construct; identical to SEQ ID NO: 32

SEQ ID NO: 41 - protein, 119 aa, synthetic construct; identical to SEQ ID NO: 27

SEQ ID NO: 42 - protein, 112 aa, synthetic construct; identical to SEQ ID NO: 32

SEQ ID NO: 43 - protein, 119 aa, synthetic construct; identical to SEQ ID NO: 29

SEQ ID NO: 44 - protein, 112 aa, synthetic construct; identical to SEQ ID NO: 32

SEQ ID NO: 45 - protein, 119 aa, synthetic construct; identical to SEQ ID NO: 17

SEQ ID NO: 46 - protein, 112 aa, synthetic construct
DIVMTQSPDSLAVSLGERATINCKSSQSLLDSDGKPYLNWLQQKPGQSPKRLIYLVSKLDSGVPDRFSGSGSGTDFTLTISSVQAEDVAVYYCWQDTHFPRTFGQGTKLEIK

SEQ ID NO: 47 - protein, 119 aa, synthetic construct; identical to SEQ ID NO: 19

SEQ ID NO: 48 - protein, 112 aa, synthetic construct; identical to SEQ ID NO: 46

SEQ ID NO: 49 - protein, 119 aa, synthetic construct; identical to SEQ ID NO: 21

SEQ ID NO: 50 - protein, 112 aa, synthetic construct; identical to SEQ ID NO: 46

SEQ ID NO: 51 - protein, 119 aa, synthetic construct; identical to SEQ ID NO: 23

SEQ ID NO: 52 - protein, 112 aa, synthetic construct; identical to SEQ ID NO: 46

SEQ ID NO: 53 - protein, 119 aa, synthetic construct; identical to SEQ ID NO: 25

SEQ ID NO: 54 - protein, 112 aa, synthetic construct; identical to SEQ ID NO: 46

SEQ ID NO: 55 - protein, 119 aa, synthetic construct; identical to SEQ ID NO: 27

SEQ ID NO: 56 - protein, 112 aa, synthetic construct; identical to SEQ ID NO: 46

SEQ ID NO: 57 - protein, 119 aa, synthetic construct; identical to SEQ ID NO: 29

SEQ ID NO: 58 - protein, 112 aa, synthetic construct; identical to SEQ ID NO: 46

SEQ ID NO: 59 - protein, 120 aa, synthetic construct
QVQLVQSGAEVKKPGASVKVSCKDSGFNIKDTYIHWVRQAPGQGLEWIGRIDPANGFTRYAQKFQGRVTMTSDTSTSTVYMELSSLRSEDTAVYYCAREDGYPYYTLDCWGQGTTVTVSS

SEQ ID NO: 60 - protein, 112 aa, synthetic construct
DVVMTQSPLSLPLTLGQPASISCRSSQSLLDVDGKTYLNWFQQRPGQSPRRLIYLVSKLDSGVPDRFSGSGSGTDFTLKISRVEAEDVGVYYCWQGTHFPRTFGQGTKLEIK

SEQ ID NO: 61 - protein, 120 aa, synthetic construct
QVQLVQSGAEVKKPGASVKVSCKDSGFNIKDTYIHWVRQAPGQGLEWIGRIDPANGFTRYAQKFQGRATITSDTSTSTVYLELSSLRSEDTAVYYCAREDGYPYYTLDCWGQGTTVTVSS

SEQ ID NO: 62 - protein, 112 aa, synthetic construct; identical to SEQ ID NO: 60

SEQ ID NO: 63 - protein, 120 aa, synthetic construct
QVQLVQSGAEVKKPGSSVKVSCKDSGFNIKDTYIHWVRQAPGQGLEWIGRIDPANGFARYAQKFQGRATITSDESTSTAYLELSSLRSEDTAVYYCAREDGYPYYTLDCWGQGTTVTVSS

SEQ ID NO: 64 - protein, 112 aa, synthetic construct; identical to SEQ ID NO: 60

SEQ ID NO: 65 - protein, 120 aa, synthetic construct
QVQLVQSGAEVKKPGSSVKVSCKDSGFNIKDTYIHWVRQAPGQGLEWIGRIDPANGFARYAQKFQGRATITSDKSTSTAYLELSSLRSEDTAVYYCAREDGYPYYTLDCWGQGTTVTVSS

SEQ ID NO: 66 - protein, 112 aa, synthetic construct; identical to SEQ ID NO: 60

SEQ ID NO: 67 - protein, 120 aa, synthetic construct
EVQLVESGGGLVQPGGSLRLSCADSGFNIKDTYIHWVRQAPGKGLEWIGRIDPANGFTRYADSVKGRFTISSDNSKNTLYLQMNSLRAEDTAVYYCAREDGYPYYTLDCWGQGTTVTVSS

SEQ ID NO: 68 - protein, 112 aa, synthetic construct; identical to SEQ ID NO: 60

SEQ ID NO: 69 - protein, 120 aa, synthetic construct
QVQLQESGPGLVKPSGTLSLTCADSGFNIKDTYIHWVRQPPGKGLEWIGRIDPANGFTRYNPSFKSRATISSDKSKNQASLKLSSVTAADTAVYYCAREDGYPYYTLDCWGQGTTVTVSS

SEQ ID NO: 70 - protein, 112 aa, synthetic construct; identical to SEQ ID NO: 60

SEQ ID NO: 71 - protein, 120 aa, synthetic construct
EVQLVQSGAEVKKPGESLKISCKDSGFNIKDTYIHWVRQMPGKGLEWIGRIDPANGFTRYSPSFQGQATISADKSISTAYLQLSSLKASDTAMYYCAREDGYPYYTLDCWGQGTTVTVSS

SEQ ID NO: 72 - protein, 112 aa, synthetic construct; identical to SEQ ID NO: 60

SEQ ID NO: 73 - protein, 120 aa, synthetic construct; identical to SEQ ID NO: 59

SEQ ID NO: 74 - protein, 112 aa, synthetic construct
EIVMTQSPATLSLSPGERATISCRSSQSLLDVDGKTYLNWFQQKPGQAPRRLIYLVSKRDSGVPARFSGSGSGTDFTLTISSVQPEDFAVYYCWQGTHFPRTFGQGTKLEIK

SEQ ID NO: 75 - protein, 120 aa, synthetic construct; identical to SEQ ID NO: 61

SEQ ID NO: 76 - protein, 112 aa, synthetic construct; identical to SEQ ID NO: 74

SEQ ID NO: 77 - protein, 120 aa, synthetic construct; identical to SEQ ID NO: 63

SEQ ID NO: 78 - protein, 112 aa, synthetic construct; identical to SEQ ID NO: 74

SEQ ID NO: 79 - protein, 120 aa, synthetic construct; identical to SEQ ID NO: 65

SEQ ID NO: 80 - protein, 112 aa, synthetic construct; identical to SEQ ID NO: 74

SEQ ID NO: 81 - protein, 120 aa, synthetic construct; identical to SEQ ID NO: 67

SEQ ID NO: 82 - protein, 112 aa, synthetic construct; identical to SEQ ID NO: 74

SEQ ID NO: 83 - protein, 120 aa, synthetic construct; identical to SEQ ID NO: 69

SEQ ID NO: 84 - protein, 112 aa, synthetic construct; identical to SEQ ID NO: 74

SEQ ID NO: 85 - protein, 120 aa, synthetic construct; identical to SEQ ID NO: 71

SEQ ID NO: 86 - protein, 112 aa, synthetic construct; identical to SEQ ID NO: 74

SEQ ID NO: 87 - protein, 120 aa, synthetic construct; identical to SEQ ID NO: 59

SEQ ID NO: 88 - protein, 112 aa, synthetic construct
DIVMTQSPDSLALSLGERATINCKSSQSLLDVDGKTYLNWFQQKPGQPPKRLIYLVSKLDSGVPDRFSGSGSGTDFTLTISSVQAEDVAVYYCWQGTHFPRTFGQGTKLEIK

SEQ ID NO: 89 - protein, 120 aa, synthetic construct; identical to SEQ ID NO: 61

SEQ ID NO: 90 - protein, 112 aa, synthetic construct; identical to SEQ ID NO: 88

SEQ ID NO: 91 - protein, 120 aa, synthetic construct; identical to SEQ ID NO: 63

SEQ ID NO: 92 - protein, 112 aa, synthetic construct; identical to SEQ ID NO: 88

SEQ ID NO: 93 - protein, 120 aa, synthetic construct; identical to SEQ ID NO: 65

SEQ ID NO: 94 - protein, 112 aa, synthetic construct; identical to SEQ ID NO: 88

SEQ ID NO: 95 - protein, 120 aa, synthetic construct; identical to SEQ ID NO: 67

SEQ ID NO: 96 - protein, 112 aa, synthetic construct; identical to SEQ ID NO: 88

SEQ ID NO: 97 - protein, 120 aa, synthetic construct; identical to SEQ ID NO: 69

SEQ ID NO: 98 - protein, 112 aa, synthetic construct; identical to SEQ ID NO: 88

SEQ ID NO: 99 - protein, 120 aa, synthetic construct; identical to SEQ ID NO: 71

SEQ ID NO: 100 - protein, 112 aa, synthetic construct; identical to SEQ ID NO: 88

SEQ ID NO: 101 - protein, 120 aa, synthetic construct
QVQLQESGPGLVKPSGTLSLTCADSGFNIKDTYIHWVRQPPGKGLEWIGRIDPANGFTRYNPSFKSRATISSDKSKNQASLKLSSVTAADTAVYYCAREDGYPYYTLDAWGQGTTVTVSS

SEQ ID NO: 102 - protein, 112 aa, synthetic construct; identical to SEQ ID NO: 60

SEQ ID NO: 103 - protein, 120 aa, synthetic construct
QVQLQESGPGLVKPSGTLSLTCADSGFNIKDTYIHWVRQPPGKGLEWIGRIDPANGFTRYNPSFKSRATISSDKSKNQASLKLSSVTAADTAVYYCAREDGYPYYTLDSWGQGTTVTVSS

SEQ ID NO: 104 - protein, 112 aa, synthetic construct; identical to SEQ ID NO: 60

SEQ ID NO: 105 - protein, 120 aa, synthetic construct
QVQLQESGPGLVKPSGTLSLTCADSGFNIKDTYIHWVRQPPGKGLEWIGRIDPANGFTRYNPSFKSRATISSDKSKNQASLKLSSVTAADTAVYYCAREDGYPYYTLDTWGQGTTVTVSS

SEQ ID NO: 106 - protein, 112 aa, synthetic construct; identical to SEQ ID NO: 60

SEQ ID NO: 107 - protein, 120 aa, synthetic construct
QVQLQESGPGLVKPSGTLSLTCADSGFNIKDTYIHWVRQPPGKGLEWIGRIDPANGFTRYNPSFKSRATISSDKSKNQASLKLSSVTAADTAVYYCAREDGYPYYTLDVWGQGTTVTVSS

SEQ ID NO: 108 - protein, 112 aa, synthetic construct; identical to SEQ ID NO: 60

SEQ ID NO: 109 - protein, 120 aa, synthetic construct; identical to SEQ ID NO: 101

SEQ ID NO: 110 - protein, 112 aa, synthetic construct; identical to SEQ ID NO: 74

SEQ ID NO: 111 - protein, 120 aa, synthetic construct; identical to SEQ ID NO: 103

SEQ ID NO: 112 - protein, 112 aa, synthetic construct; identical to SEQ ID NO: 74

SEQ ID NO: 113 - protein, 120 aa, synthetic construct; identical to SEQ ID NO: 105

SEQ ID NO: 114 - protein, 112 aa, synthetic construct; identical to SEQ ID NO: 74

SEQ ID NO: 115 - protein, 120 aa, synthetic construct; identical to SEQ ID NO: 107

SEQ ID NO: 116 - protein, 112 aa, synthetic construct; identical to SEQ ID NO: 74

SEQ ID NO: 117 - protein, 120 aa, synthetic construct; identical to SEQ ID NO: 101

SEQ ID NO: 118 - protein, 112 aa, synthetic construct; identical to SEQ ID NO: 88

SEQ ID NO: 119 - protein, 120 aa, synthetic construct; identical to SEQ ID NO: 103

SEQ ID NO: 120 - protein, 112 aa, synthetic construct; identical to SEQ ID NO: 88

SEQ ID NO: 121 - protein, 120 aa, synthetic construct; identical to SEQ ID NO: 105

SEQ ID NO: 122 - protein, 112 aa, synthetic construct; identical to SEQ ID NO: 88

SEQ ID NO: 123 - protein, 120 aa, synthetic construct; identical to SEQ ID NO: 107

SEQ ID NO: 124 - protein, 112 aa, synthetic construct; identical to SEQ ID NO: 88

SEQ ID NO: 125 - protein, 120 aa, synthetic construct
EVQLVESGGGLVQPGGSLRLSCADSGFTIKDTYIHWVRQAPGKGLEWVGRIDPANGFTRYADSVKGRFTISSDNSKNTLYLQMNSLRAEDTAVYYCAREDGYPYYTLDAWGQGTTVTVSS

SEQ ID NO: 126 - protein, 112 aa, synthetic construct; identical to SEQ ID NO: 88

SEQ ID NO: 127 - protein, 120 aa, synthetic construct
EVQLVESGGGLVQPGGSLRLSCAASGFTIKDTYIHWVRQAPGKGLEWVGRIDSANGFTRYADSVKGRFTISSDNSKNTLYLQMNSLRAEDTAVYYCAREDGYPYYTLDAWGQGTTVTVSS

SEQ ID NO: 128 - protein, 112 aa, synthetic construct; identical to SEQ ID NO: 88

SEQ ID NO: 129 - protein, 120 aa, synthetic construct
QVQLQESGPGLVKPSGTLSLTCADSGFNIKDTYIHWVRQPPGKGLEWIGRIDPANGFTRYNPSLKSRVTISSDKSKNQASLKLSSVTAADTAVYYCAREDGYPYYTLDAWGQGTTVTVSS

SEQ ID NO: 130 - protein, 112 aa, synthetic construct; identical to SEQ ID NO: 88

SEQ ID NO: 131 - protein, 120 aa, synthetic construct
QVQLQESGPGLVKPSGTLSLTCADSGFSISDTYIHWVRQPPGKGLEWIGRIDPANGFTRYNPSLKSRVTISSDKSKNQASLKLSSVTAADTAVYYCAREDGYPYYTLDAWGQGTTVTVSS

SEQ ID NO: 132 - protein, 112 aa, synthetic construct; identical to SEQ ID NO: 88

SEQ ID NO: 133 - protein, 120 aa, synthetic construct
QVQLQESGPGLVKPSGTLSLTCAVSGFSISDTYIHWVRQPPGKGLEWIGRIDPANGFTRYNPSLKSRVTISVDKSKNQASLKLSSVTAADTAVYYCAREDGYPYYTLDAWGQGTTVTVSS

SEQ ID NO: 134 - protein, 112 aa, synthetic construct; identical to SEQ ID NO: 88

SEQ ID NO: 135 - protein, 120 aa, synthetic construct
QVQLQESGPGLVKPSGTLSLTCAVSGFSISDTYIHWVRQPPGKGLEWIGRIDSANGFTRYNPSLKSRVTISVDKSKNQASLKLSSVTAADTAVYYCAREDGYPYYTLDAWGQGTTVTVSS

SEQ ID NO: 136 - protein, 112 aa, synthetic construct; identical to SEQ ID NO: 88

SEQ ID NO: 137 - protein, 120 aa, synthetic construct
EVQLVESGGGLVQPGGSLRLSCADSGFTIKDTYIHWVRQAPGKGLEWVGRIDPANGFTRYADSVKGRFTISSDNSKNTLYLQMNSLRAEDTAVYYCAREDGYPYYTLDSWGQGTTVTVSS

SEQ ID NO: 138 - protein, 112 aa, synthetic construct; identical to SEQ ID NO: 88

SEQ ID NO: 139 - protein, 120 aa, synthetic construct
EVQLVESGGGLVQPGGSLRLSCAASGFTIKDTYIHWVRQAPGKGLEWVGRIDSANGFTRYADSVKGRFTISSDNSKNTLYLQMNSLRAEDTAVYYCAREDGYPYYTLDSWGQGTTVTVSS

SEQ ID NO: 140 - protein, 112 aa, synthetic construct; identical to SEQ ID NO: 88

SEQ ID NO: 141 - protein, 120 aa, synthetic construct
QVQLQESGPGLVKPSGTLSLTCADSGFNIKDTYIHWVRQPPGKGLEWIGRIDPANGFTRYNPSLKSRVTISSDKSKNQASLKLSSVTAADTAVYYCAREDGYPYYTLDSWGQGTTVTVSS

SEQ ID NO: 142 - protein, 112 aa, synthetic construct; identical to SEQ ID NO: 88

SEQ ID NO: 143 - protein, 120 aa, synthetic construct
QVQLQESGPGLVKPSGTLSLTCADSGFSISDTYIHWVRQPPGKGLEWIGRIDPANGFTRYNPSLKSRVTISSDKSKNQASLKLSSVTAADTAVYYCAREDGYPYYTLDSWGQGTTVTVSS

SEQ ID NO: 144 - protein, 112 aa, synthetic construct; identical to SEQ ID NO: 88

SEQ ID NO: 145 - protein, 120 aa, synthetic construct
QVQLQESGPGLVKPSGTLSLTCAVSGFSISDTYIHWVRQPPGKGLEWIGRIDPANGFTRYNPSLKSRVTISVDKSKNQASLKLSSVTAADTAVYYCAREDGYPYYTLDSWGQGTTVTVSS

SEQ ID NO: 146 - protein, 112 aa, synthetic construct; identical to SEQ ID NO: 88

SEQ ID NO: 147 - protein, 120 aa, synthetic construct
QVQLQESGPGLVKPSGTLSLTCAVSGFSISDTYIHWVRQPPGKGLEWIGRIDSANGFTRYNPSLKSRVTISVDKSKNQASLKLSSVTAADTAVYYCAREDGYPYYTLDSWGQGTTVTVSS

SEQ ID NO: 148 - protein, 112 aa, synthetic construct; identical to SEQ ID NO: 88

SEQ ID NO: 149 - protein, 15 aa, synthetic construct
CAREDGYPYYTLDAW

SEQ ID NO: 150 - protein, 15 aa, synthetic construct
CAREDGYPYYTLDSW

SEQ ID NO: 151 - protein, 15 aa, synthetic construct
CAREDGYPYYTLDTW

SEQ ID NO: 152 - protein, 15 aa, synthetic construct
CAREDGYPYYTLDVW

SEQ ID NO: 153 - protein, 15 aa, synthetic construct
  VARIANT 14: note = X at position 14 can be a C, A, S, T, or V
CAREDGYPYYTLDXW

(19) United States
(12) Patent Application Publication
(10) Pub. No.: US 2025/0304693 A1
(43) Pub. Date: Oct. 2, 2025

(54) ANTI-BTLA ANTIBODIES AND USES THEREOF IN TREATING CANCER

(71) Applicant: HiFiBiO, Inc., Cambridge, MA (US)

(72) Inventors: Juying LI, Cambridge, MA (US); Qian ZHANG, Cambridge, MA (US); Bingqing SHEN, Hangzhou (CN); Francisco ADRIAN, Cambridge, MA (US); Liang SCHWEIZER, Cambridge, MA (US)

(21) Appl. No.: 18/833,858

(22) PCT Filed: Jan. 29, 2023
(86) PCT No.: PCT/CN2023/073700

§ 371 Date: Jul. 26, 2024

(30) Foreign Application Priority Data
Jan. 29, 2022 (WO) PCT/CN2022/075097

(51) Int. Cl. C07K 16/28; A61K 39/00; G01N 33/574

(52) U.S. Cl. CPC C07K 16/2818; G01N 33/57492; A61K 2039/505; C07K 2317/31; C07K 2317/33; C07K 2317/565; C07K 2317/73; C07K 2317/75; C07K 2317/76; G01N 2333/70521

(57) ABSTRACT

Anti-BTLA antibodies and antigen-binding fragments thereof are described. Also described are nucleic acids encoding the antibodies, compositions comprising the antibodies, and methods of producing the antibodies and using the antibodies for treating or preventing cancer in a subject in need thereof.

### CROSS REFERENCE TO RELATED APPLICATIONS

This application is a section 371 of International Application No. PCT/CN2023/073700 filed on Jan. 29, 2023, which was published in the English language on Aug. 3, 2023, under International Publication No. WO 2023/143565 A1, and which claims priority to PCT Patent Application No. PCT/CN2022/075097, filed on Jan. 29, 2022. Each disclosure o is incorporated herein by reference in its entirety.

### FIELD OF THE INVENTION

This invention relates to isolated anti-B- and T-lymphocyte attenuator (BTLA) monoclonal antibodies or antigen-binding fragments thereof, nucleic acids and expression vectors encoding the antibodies, recombinant cells containing the vectors, and compositions comprising the antibodies. Methods of making the antibodies, and methods of using the antibodies to treat diseases including cancer and/or associated complications are also provided.

### REFERENCE TO SEQUENCE LISTING SUBMITTED ELECTRONICALLY

This application contains a sequence listing, which is submitted electronically. The contents of the electronic sequence listing (065798.8US1 Sequence Listing.xml; size: 148,881 bytes; and creation date of Feb. 10, 2025) is herein incorporated by reference in its entirety.

### BACKGROUND OF THE INVENTION

B- and T-lymphocyte attenuator (BTLA) belongs to the CD28 immunoglobulin superfamily. It is a co-inhibitory immune checkpoint molecule and shares structural and functional similarity with PD-1 and CTLA-4. The expression of BTLA is limited to immune cells (T cells, B cell, DCs, NK cell, etc). Upon its ligand, HVEM, binding, the intracellular domain of BTLA signals through two phosphatases, SHPI and SHP2, to inhibit downstream TCR and BCR signaling in T cell and B cells. BTLA has a different expression profile compared to other inhibitory checkpoints during T cell differentiation, suggesting that blocking multiple inhibitory molecules simultaneously or sequentially may improve T-cell based therapy. Therefore, BTLA is an ideal target for cancer immunotherapies to treat and potentially cure BTLA-positive cancers.

### BRIEF SUMMARY OF THE INVENTION

In one general aspect, the invention relates to isolated monoclonal antibodies or antigen-binding fragments thereof that specifically bind B- and T-lymphocyte attenuator (BTLA).

Provided are isolated monoclonal antibodies or antigen-binding fragments thereof comprising a heavy chain complementarity determining region 1 (HCDR1), HCDR2, HCDR3, a light chain complementarity determining region 1 (LCDR1), LCDR2, and LCDR3, having the polypeptide sequences of;
(1) SEQ ID NOs: 7, 8, 153, 10, 11, and 12, respectively: or
(2) SEQ ID NOs: 1, 2, 3, 4, 5, and 6, respectively:

wherein the antibody or antigen-binding fragment thereof specifically binds B- and T-lymphocyte attenuator (BTLA), preferably human BTLA, optionally, said monoclonal antibody or antigen-binding fragment thereof is not naturally occurring. SEQ ID NO:153 is represented by the amino acid sequence CAREDGYPYYTLDX₁W, wherein X₁ is an amino acid selected from C, A, S, T, or V.

In certain embodiments, the isolated monoclonal antibodies or antigen-binding fragments thereof comprise a HCDR1, HCDR2, HCDR3, LCDR1, LCDR2, and LCDR3, having the polypeptide sequences of;
(1) SEQ ID NOs: 7, 8, 9, 10, 11, and 12, respectively: or
(2) SEQ ID NOs: 1, 2, 3, 4, 5, and 6, respectively;
   wherein the antibody or antigen-binding fragment thereof specifically binds B- and T-lymphocyte attenuator (BTLA), preferably human BTLA, optionally, said monoclonal antibody or antigen-binding fragment thereof is not naturally occurring.

In certain embodiments, the isolated monoclonal antibody or antigen-binding fragment thereof comprises a heavy chain variable region having a polypeptide sequence at least 90%, at least 91%, at least 92%, at least 93%, at least 94%, at least 95%, at least 96%, at least 97%, at least 98%, or at least 99% identical to SEQ ID NO:15 or 13, or a light chain variable region having a polypeptide sequence at least 90%, at least 91%, at least 92%, at least 93%, at least 94%, at least 95%, at least 96%, at least 97%, at least 98%, or at least 99% identical to SEQ ID NO:16 or 14.

In certain embodiments, the isolated anti-BTLA monoclonal antibody or antigen-binding fragment thereof comprises;
(1) a heavy chain variable region having the polypeptide sequence of SEQ ID NO:15, and a light chain variable region having the polypeptide sequence of SEQ ID NO:16; or
(2) a heavy chain variable region having the polypeptide sequence of SEQ ID NO:13, and a light chain variable region having the polypeptide sequence of SEQ ID NO:14.

In certain embodiments, the isolated monoclonal antibody or antigen-binding fragment thereof is chimeric or human or humanized.

In certain embodiments, the humanized monoclonal antibody or antigen-binding fragment thereof comprises a heavy chain complementarity determining region 1 (HCDR1), HCDR2, HCDR3, a light chain complementarity determining region 1 (LCDR1), LCDR2, and LCDR3, having the polypeptide sequences of;
(1) SEQ ID NOs: 7, 8, 150, 10, 11, and 12, respectively;
(2) SEQ ID NOs: 1, 2, 3, 4, 5, and 6, respectively;
(3) SEQ ID NOs: 7, 8, 9, 10, 11, and 12, respectively;
(4) SEQ ID NOs: 7, 8, 149, 10, 11, and 12, respectively;
(5) SEQ ID NOs: 7, 8, 151, 10, 11, and 12, respectively: or
(6) SEQ ID NOs: 7, 8, 152, 10, 11, and 12, respectively.

In certain embodiments, the isolated monoclonal antibody or antigen-binding fragment thereof comprises a heavy chain variable region having a polypeptide sequence at least 90%, at least 91%, at least 92%, at least 93%, at least 94%, at least 95%, at least 96%, at least 97%, at least 98%, or at least 99% identical to SEQ ID NO:141, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 143, 145, or 147, or a light chain variable region having a polypeptide sequence at least 90%, at least 91%, at least 92%, at least 93%, at least 94%, at least 95%, at least 96%, at least 97%, at least 98%, or at least 99% identical to SEQ ID NO:142, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 144, 146, or 148.

In certain embodiments, the isolated humanized anti-BTLA monoclonal antibody or antigen-binding fragment thereof comprises;
(1) a heavy chain variable region having the polypeptide sequence of SEQ ID NO:141, and a light chain variable region having the polypeptide sequence of SEQ ID NO:142;
(2) a heavy chain variable region having the polypeptide sequence of SEQ ID NO:17, and a light chain variable region having the polypeptide sequence of SEQ ID NO:18;
(3) a heavy chain variable region having the polypeptide sequence of SEQ ID NO:19, and a light chain variable region having the polypeptide sequence of SEQ ID NO:20;
(4) a heavy chain variable region having the polypeptide sequence of SEQ ID NO:21, and a light chain variable region having the polypeptide sequence of SEQ ID NO:22;
(5) a heavy chain variable region having the polypeptide sequence of SEQ ID NO:23, and a light chain variable region having the polypeptide sequence of SEQ ID NO:24;
(6) a heavy chain variable region having the polypeptide sequence of SEQ ID NO:25, and a light chain variable region having the polypeptide sequence of SEQ ID NO:26;
(7) a heavy chain variable region having the polypeptide sequence of SEQ ID NO:27, and a light chain variable region having the polypeptide sequence of SEQ ID NO:28;
(8) a heavy chain variable region having the polypeptide sequence of SEQ ID NO:29, and a light chain variable region having the polypeptide sequence of SEQ ID NO:30;
(9) a heavy chain variable region having the polypeptide sequence of SEQ ID NO:31, and a light chain variable region having the polypeptide sequence of SEQ ID NO:32;
(10) a heavy chain variable region having the polypeptide sequence of SEQ ID NO:33, and a light chain variable region having the polypeptide sequence of SEQ ID NO:34;
(11) a heavy chain variable region having the polypeptide sequence of SEQ ID NO:35, and a light chain variable region having the polypeptide sequence of SEQ ID NO:36;
(12) a heavy chain variable region having the polypeptide sequence of SEQ ID NO:37, and a light chain variable region having the polypeptide sequence of SEQ ID NO:38;
(13) a heavy chain variable region having the polypeptide sequence of SEQ ID NO:39, and a light chain variable region having the polypeptide sequence of SEQ ID NO:40;
(14) a heavy chain variable region having the polypeptide sequence of SEQ ID NO:41, and a light chain variable region having the polypeptide sequence of SEQ ID NO:42;
(15) a heavy chain variable region having the polypeptide sequence of SEQ ID NO:43, and a light chain variable region having the polypeptide sequence of SEQ ID NO:44;
(16) a heavy chain variable region having the polypeptide sequence of SEQ ID NO:45, and a light chain variable region having the polypeptide sequence of SEQ ID NO:46;
(17) a heavy chain variable region having the polypeptide sequence of SEQ ID NO:47, and a light chain variable region having the polypeptide sequence of SEQ ID NO:48;
(18) a heavy chain variable region having the polypeptide sequence of SEQ ID NO:49, and a light chain variable region having the polypeptide sequence of SEQ ID NO:50;
(19) a heavy chain variable region having the polypeptide sequence of SEQ ID NO:51, and a light chain variable region having the polypeptide sequence of SEQ ID NO:52;
(20) a heavy chain variable region having the polypeptide sequence of SEQ ID NO:53, and a light chain variable region having the polypeptide sequence of SEQ ID NO:54;
(21) a heavy chain variable region having the polypeptide sequence of SEQ ID NO:55, and a light chain variable region having the polypeptide sequence of SEQ ID NO:56;
(22) a heavy chain variable region having the polypeptide sequence of SEQ ID NO:57, and a light chain variable region having the polypeptide sequence of SEQ ID NO:58;
(23) a heavy chain variable region having the polypeptide sequence of SEQ ID NO:59, and a light chain variable region having the polypeptide sequence of SEQ ID NO:60;
(24) a heavy chain variable region having the polypeptide sequence of SEQ ID NO:61, and a light chain variable region having the polypeptide sequence of SEQ ID NO:62;
(25) a heavy chain variable region having the polypeptide sequence of SEQ ID NO:63, and a light chain variable region having the polypeptide sequence of SEQ ID NO:64;
(26) a heavy chain variable region having the polypeptide sequence of SEQ ID NO:65, and a light chain variable region having the polypeptide sequence of SEQ ID NO:66;
(27) a heavy chain variable region having the polypeptide sequence of SEQ ID NO:67, and a light chain variable region having the polypeptide sequence of SEQ ID NO:68;
(28) a heavy chain variable region having the polypeptide sequence of SEQ ID NO:69, and a light chain variable region having the polypeptide sequence of SEQ ID NO:70;
(29) a heavy chain variable region having the polypeptide sequence of SEQ ID NO:71, and a light chain variable region having the polypeptide sequence of SEQ ID NO:72;
(30) a heavy chain variable region having the polypeptide sequence of SEQ ID NO:73, and a light chain variable region having the polypeptide sequence of SEQ ID NO:74;
(31) a heavy chain variable region having the polypeptide sequence of SEQ ID NO:75, and a light chain variable region having the polypeptide sequence of SEQ ID NO:76;
(32) a heavy chain variable region having the polypeptide sequence of SEQ ID NO:77, and a light chain variable region having the polypeptide sequence of SEQ ID NO:78;
(33) a heavy chain variable region having the polypeptide sequence of SEQ ID NO:79, and a light chain variable region having the polypeptide sequence of SEQ ID NO:80;
(34) a heavy chain variable region having the polypeptide sequence of SEQ ID NO:81, and a light chain variable region having the polypeptide sequence of SEQ ID NO:82;
(35) a heavy chain variable region having the polypeptide sequence of SEQ ID NO:83, and a light chain variable region having the polypeptide sequence of SEQ ID NO:84;
(36) a heavy chain variable region having the polypeptide sequence of SEQ ID NO:85, and a light chain variable region having the polypeptide sequence of SEQ ID NO:86;
(37) a heavy chain variable region having the polypeptide sequence of SEQ ID NO:87, and a light chain variable region having the polypeptide sequence of SEQ ID NO:88;
(38) a heavy chain variable region having the polypeptide sequence of SEQ ID NO:89, and a light chain variable region having the polypeptide sequence of SEQ ID NO:90;
(39) a heavy chain variable region having the polypeptide sequence of SEQ ID NO:91, and a light chain variable region having the polypeptide sequence of SEQ ID NO:92;
(40) a heavy chain variable region having the polypeptide sequence of SEQ ID NO:93, and a light chain variable region having the polypeptide sequence of SEQ ID NO:94;
(41) a heavy chain variable region having the polypeptide sequence of SEQ ID NO:95, and a light chain variable region having the polypeptide sequence of SEQ ID NO:96;
(42) a heavy chain variable region having the polypeptide sequence of SEQ ID NO:97, and a light chain variable region having the polypeptide sequence of SEQ ID NO:98;
(43) a heavy chain variable region having the polypeptide sequence of SEQ ID NO:99, and a light chain variable region having the polypeptide sequence of SEQ ID NO:100;
(44) a heavy chain variable region having the polypeptide sequence of SEQ ID NO:101, and a light chain variable region having the polypeptide sequence of SEQ ID NO:102;
(45) a heavy chain variable region having the polypeptide sequence of SEQ ID NO:103, and a light chain variable region having the polypeptide sequence of SEQ ID NO:104;
(46) a heavy chain variable region having the polypeptide sequence of SEQ ID NO:105, and a light chain variable region having the polypeptide sequence of SEQ ID NO:106;
(47) a heavy chain variable region having the polypeptide sequence of SEQ ID NO:107, and a light chain variable region having the polypeptide sequence of SEQ ID NO:108;
(48) a heavy chain variable region having the polypeptide sequence of SEQ ID NO:109, and a light chain variable region having the polypeptide sequence of SEQ ID NO:110;
(49) a heavy chain variable region having the polypeptide sequence of SEQ ID NO:111, and a light chain variable region having the polypeptide sequence of SEQ ID NO:112;
(50) a heavy chain variable region having the polypeptide sequence of SEQ ID NO:113, and a light chain variable region having the polypeptide sequence of SEQ ID NO:114;
(51) a heavy chain variable region having the polypeptide sequence of SEQ ID NO:115, and a light chain variable region having the polypeptide sequence of SEQ ID NO:116;
(52) a heavy chain variable region having the polypeptide sequence of SEQ ID NO:117, and a light chain variable region having the polypeptide sequence of SEQ ID NO:118;
(53) a heavy chain variable region having the polypeptide sequence of SEQ ID NO:119, and a light chain variable region having the polypeptide sequence of SEQ ID NO:120;
(54) a heavy chain variable region having the polypeptide sequence of SEQ ID NO:121, and a light chain variable region having the polypeptide sequence of SEQ ID NO:122;
(55) a heavy chain variable region having the polypeptide sequence of SEQ ID NO:123, and a light chain variable region having the polypeptide sequence of SEQ ID NO:124;
(56) a heavy chain variable region having the polypeptide sequence of SEQ ID NO:125, and a light chain variable region having the polypeptide sequence of SEQ ID NO:126;
(57) a heavy chain variable region having the polypeptide sequence of SEQ ID NO:127, and a light chain variable region having the polypeptide sequence of SEQ ID NO:128;
(58) a heavy chain variable region having the polypeptide sequence of SEQ ID NO:129, and a light chain variable region having the polypeptide sequence of SEQ ID NO:130;
(59) a heavy chain variable region having the polypeptide sequence of SEQ ID NO:131, and a light chain variable region having the polypeptide sequence of SEQ ID NO:132;
(60) a heavy chain variable region having the polypeptide sequence of SEQ ID NO:133, and a light chain variable region having the polypeptide sequence of SEQ ID NO:134;
(61) a heavy chain variable region having the polypeptide sequence of SEQ ID NO:135, and a light chain variable region having the polypeptide sequence of SEQ ID NO:136;
(62) a heavy chain variable region having the polypeptide sequence of SEQ ID NO:137, and a light chain variable region having the polypeptide sequence of SEQ ID NO:138;
(63) a heavy chain variable region having the polypeptide sequence of SEQ ID NO:139, and a light chain variable region having the polypeptide sequence of SEQ ID NO:140;
(64) a heavy chain variable region having the polypeptide sequence of SEQ ID NO:143, and a light chain variable region having the polypeptide sequence of SEQ ID NO:144;
(65) a heavy chain variable region having the polypeptide sequence of SEQ ID NO:145, and a light chain variable region having the polypeptide sequence of SEQ ID NO:146: or
(66) a heavy chain variable region having the polypeptide sequence of SEQ ID NO:147, and a light chain variable region having the polypeptide sequence of SEQ ID NO:148.

In certain embodiments, the isolated monoclonal antibody or antigen-binding fragment thereof specifically binds cynomolgus BTLA.

In certain embodiments, the isolated monoclonal antibody or antigen-binding fragment thereof binds to human BTLA with a K_{D} of less than about 25 nM, 20 nM, 15 nM, 5 nM, 2 nM, 1 nM, or 0.5 nM.

In certain embodiments, the isolated monoclonal antibody or antigen-binding fragment thereof is an agonist of human BTLA and activates downstream signaling from BTLA upon binding to BTLA.

In certain embodiments, the monoclonal antibody or antigen-binding fragment thereof is capable of inhibiting B cell proliferation and/or T cell and/or plasma cell activation. The T-cell can, for example, be selected from the group consisting of a CD4 T-cell, a CD8 T-cell, a Th1 T-cell, a TFH T-cell, an αβ T-cell, and a γδ T-cell.

In certain embodiments, the monoclonal antibody or antigen-binding fragment thereof is capable of blocking or abolishing BTLA binding to herpes virus entry mediator (HVEM).

In certain embodiments, the isolated monoclonal antibody or antigen-binding fragment thereof binds to BTLA and is capable of increasing pro-inflammatory cytokine production, such as, for example, interferon γ (IFNγ), interleukin-2 (IL-2), C—X—C motif chemokine ligand 9 (CXCL9), C—X—C motif chemokine ligand 10 (CXCL10), interleukin 1β (IL1β), and tumor necrosis factor α (TNFα): mediating the activity of conjugated drugs; and/or forming a bispecific antibody with another monoclonal antibody or antigen-binding fragment thereof with cancer-killing effect.

Also provided are isolated bispecific antibodies or antigen-binding fragments thereof comprising the monoclonal antibodies or antigen-binding fragments thereof of the invention.

Also provided are isolated nucleic acids encoding the monoclonal antibodies or antigen-binding fragments thereof or bispecific antibodies or antigen-binding fragments thereof of the invention.

Also provided are vectors comprising the isolated nucleic acids encoding the monoclonal antibodies or antigen-binding fragments thereof or bispecific antibodies or antigen-binding fragments thereof of the invention.

Also provided are host cells comprising the vectors comprising the isolated nucleic acids encoding the monoclonal antibodies or antigen-binding fragments thereof or bispecific antibodies or antigen-binding fragments thereof of the invention.

In certain embodiments, provided is a pharmaceutical composition comprising an isolated monoclonal antibody or antigen-binding fragment thereof or an isolated bispecific antibody or antigen-binding fragment thereof of the invention and a pharmaceutically acceptable carrier.

Also provided are methods of specifically targeting B- and T-lymphocyte attenuator (BTLA) on a cancer cell surface in a subject in need thereof, comprising administering to the subject a pharmaceutical composition of the invention.

Also provided are methods of treating cancer in a subject in need thereof, comprising administering to the subject the pharmaceutical compositions of the invention. In certain embodiments, the cancer is a solid tumor, preferably a solid tumor with infiltrating T cells, more preferably a solid tumor with infiltrating T effector cells, more preferably a solid tumor with T effector cells expressing BTLA, most preferably a solid tumor with infiltrating T effector cells expressing BTLA and BTLA ligand HVEM highly expressing in the tumor microenvironment. The cancer can, for example, be a BTLA-positive cancer. Examples of cancers can, for example, be selected from, but not limited to, melanoma, lung cancer, renal cell carcinoma, and liver cancer.

In certain embodiments, the pharmaceutical composition further comprises a second therapeutic anti-cancer agent. The second therapeutic anti-cancer agent can, for example, be an anti-PD1 antibody or antigen-binding fragment thereof.

Also provided are methods of producing a monoclonal antibody or antigen-binding fragment thereof or bispecific antibody or antigen-binding fragment thereof of the invention. The methods comprise culturing a cell comprising a nucleic acid encoding the monoclonal antibody or antigen-binding fragment thereof or bispecific antibody or antigen-binding fragment thereof under conditions to produce the monoclonal antibody or antigen-binding fragment thereof or bispecific antibody or antigen-binding fragment thereof, and recovering the monoclonal antibody or antigen-binding fragment thereof or bispecific antibody or antigen-binding fragment thereof from the cell or culture.

Also provided are methods of producing a pharmaceutical composition comprising a monoclonal antibody or antigen-binding fragment thereof or bispecific antibody or antigen-binding fragment thereof of the invention. The methods comprise combining the monoclonal antibody or antigen-binding fragment thereof or bispecific antibody or antigen-binding fragment thereof with a pharmaceutically acceptable carrier to obtain the pharmaceutical composition.

Also provided are methods of determining the level of BTLA in a subject. The methods comprise (a) obtaining a sample from the subject: (b) contacting the sample with an anti-BTLA monoclonal antibody or antigen-binding fragment thereof of the invention; and (c) determining the level of BTLA in the subject. In certain embodiments, the sample is a tissue sample. The tissue sample can, for example, be a cancer tissue sample. In certain embodiments, the sample is a blood sample. In certain embodiments, the sample comprise T cells (e.g., CD4, CD8, Th1, TFH, αβ, γδ), B cells, dendritic cells (DCs), and natural killer (NK) cells.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B show graphs demonstrating identification of anti-BTLA blocking antibodies HFB6-2 and HFB6-3 through the HVEM-BTLA blockade reporter assay.

FIGS. 2A-2B show graphs demonstrating that HFB6-2 and HFB6-3 antibodies are capable of binding human and cynomolgus BTLA in HEK293 human BTLA (FIG. 2A) and Expi293 cynomolgus BTLA (FIG. 2B) in FACS binding assays.

FIGS. 3A-3B show graphs demonstrating that HFB6-2 and HFB6-3 antibodies are anti-BTLA blockers in cellular BTLA/soluble HVEM blocking (FIG. 3A) and BTLA-HVEM blockade T cell reporter (FIG. 3B) assay's.

FIGS. 4A-4D show graphs demonstrating HFB6-2 and HFB6-3 antibodies bind to primary T and B cells.

FIG. 5 shows a graph demonstrating HFB6-2 and HFB6-3 antibodies reverse HVEM-mediated suppression on primary CD4+ T-cell proliferation.

FIG. 6 shows graphs demonstrating HFB6-3 increased IFNγ production and synergized with anti-PD1 in a mixed lymphocyte reaction (MLR) assay.

FIGS. 7A-7G show graphs demonstrating HFB6-3 antibodies, alone or in combination with anti-PD1 antibodies, induced IFNγ and pro-inflammatory cytokine production in primary dissociated tumor cultures.

FIGS. 8A-8B show graphs demonstrating desirable PK profiles for HFB6-2 and HFB6-3 antibodies in wild-type C57BL/6 mice and hBTLA knock-in mice.

FIG. 9 shows graphs demonstrating functional profiling of HFB6-2 humanized antibodies in an ELISA binding assay and an HVEM-BTLA blockade reporter assay.

FIGS. 10A-10C show graphs demonstrating the results of humanized HFB6-3 antibodies (including Cys mutants in the HCDR3 of the humanized HFB6-3 antibodies) in the HVEM-BTLA blockade reporter assay.

FIGS. 11A-11B show graphs demonstrating that humanized HFB6-3 antibody variants are capable of binding human and cynomolgus BTLA in HEK293T human BTLA expressing cells (FIG. 11A) and Expi293 cynomolgus BTLA expressing cells (FIG. 11B) in FACS binding assays.

FIGS. 12A-12B show graphs demonstrating that humanized HFB6-3 antibody variants are capable of blocking cellular BTLA and soluble HVEM interaction in FACS blocking assay (FIG. 12A) and in the CHO.TCRa.hHVEM/Jurkat.hBTLA.NFAT-luc blockade reporter assay (FIG. 12B).

FIG. 13 shows a graph demonstrating that humanized HFB6-3 antibody variants with cysteine mutations were capable of reversing HVEM-mediated suppression on primary CD4+ T-cell proliferation.

FIGS. 14A-14C show graphs demonstrating that humanized HFB6-3 antibody variants with cysteine mutations induced IFNγ and pro-inflammatory cytokine production in primary dissociated tumor cultures.

FIGS. 15A-15C show graphs demonstrating that humanized HFB6-3 antibody variants with cysteine mutations induced IFNγ and pro-inflammatory cytokine production in primary dissociated tumor cultures.

### DETAILED DESCRIPTION OF THE INVENTION

Various publications, articles and patents are cited or described in the background and throughout the specification: each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of.” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally.” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity.” in the context of two or more nucleic acids or polypeptide sequences (e.g., anti-BTLA antibodies and polynucleotides that encode them. BTLA polypeptides and BTLA polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman. Adv. Appl. Math. 1981; 2:482, by the homology alignment algorithm of Needleman & Wunsch. J. Mol. Biol. 1970; 48:443, by the search for similarity method of Pearson & Lipman. Proc. Nat'l. Acad. Sci. USA 1988; 85:2444, by computerized implementations of these algorithms (GAP. BESTFIT. FASTA, and TFASTA in the Wisconsin Genetics Software Package. Genetics Computer Group. 575 Science Dr., Madison, WI), or by visual inspection (see generally. Current Protocols in Molecular Biology. F. M. Ausubel et al., eds., Current Protocols, a joint venture between Greene Publishing Associates, Inc, and John Wiley & Sons. Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215: 403-410 and Altschul et al., Nucleic Acids Res. 1997: 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues: always >0) and N (penalty score for mismatching residues: always <0). For amino acid sequences, a scoring matrix is used to calculate the cumulative score. Extension of the word hits in each direction are halted when: the cumulative alignment score falls off by the quantity X from its maximum achieved value: the cumulative score goes to zero or below, due to the accumulation of one or more negative-scoring residue alignments: or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs. i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide.” synonymously referred to as “nucleic acid molecule.” “nucleotides” or “nucleic acids.” refers to any polyribonucleotide or polydeoxyribonucleotide, which can be unmodified RNA or DNA or modified RNA or DNA. “Polynucleotides” include, without limitation single- and double-stranded DNA. DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition. “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus. “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide.” “polypeptide.” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention: for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### Antibodies

The invention generally relates to isolated B- and T-lymphocyte attenuator (BTLA) antibodies, nucleic acids and expression vectors encoding the antibodies, recombinant cells containing the vectors, recombinant cells expressing the antibodies, and compositions comprising the antibodies. Methods of making the antibodies, and methods of using the antibodies to treat diseases, such as cancer, are also disclosed. The antibodies of the invention possess one or more desirable functional properties, including, but not limited to, high-affinity binding to BTLA, high specificity to BTLA, the ability to block binding of herpes virus entry mediator (HVEM) and BTLA, the ability to inhibit B cell proliferation and/or T-cell and/or plasma cell activation, the ability to increase pro-inflammatory cytokine production, and the ability to inhibit tumor growth in subjects and animal models when administered alone or in combination with other anti-cancer therapies. Pro-inflammatory cytokines can include, but are not limited to, interleukin 2 (IL-2). C—X—C motif chemokine ligand 9 (CXCL9). C—X—C motif chemokine ligand 10 (CXCL10), interleukin 1β (IL1β), tumor necrosis factor α (TNFα), and interferon γ. T-cells can include, but are not limited to, CD4 T-cells, CD8 T-cells, Th1 T-cells, TFH T-cells, αβ T-cells, and γδ T-cells.

In a general aspect, the invention relates to isolated monoclonal antibodies or antigen-binding fragments thereof that bind B- and T-lymphocyte attenuator (BTLA).

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

“Complementarity determining regions” (CDR) are antibody regions that bind an antigen. CDRs may be defined using various delineations such as Kabat (Wu et al. J Exp Med 132: 211-50, 1970) (Kabat et al., Sequences of Proteins of Immunological Interest. 5th Ed. Public Health Service, National Institutes of Health. Bethesda. Md., 1991). Chothia (Chothia et al. J Mol Biol 196:901-17, 1987), IMGT (Lefranc et al. Dev Comp Immunol 27: 55-77, 2003) and AbM (Martin and Thornton J Bmol Biol 263:800-15, 1996). The correspondence between the various delineations and variable region numbering are described (see e.g., Lefranc et al. Dev Comp Immunol 27:55-77, 2003: Honegger and Pluckthun, J Mol Biol 309:657-70, 2001; International ImMunoGeneTics (IMGT) database: Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat: Chothia: Martin: Lefranc et al.).

TABLE 1

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

As used herein, the term an “isolated antibody” refers to an antibody which is substantially free of other antibodies having different antigenic specificities (e.g., an isolated antibody that specifically binds to BTLA is substantially free of antibodies that do not bind to BTLA). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids.

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” and/or “humanized antigen binding domain” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multi-specific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., different proteins (or different subunits of a multimeric protein). In an embodiment, a multi-specific antibody comprises a third, fourth, or fifth immunoglobulin variable domain. In an embodiment, a multi-specific antibody is a bispecific antibody molecule, a tri-specific antibody molecule, or a tetra-specific antibody molecule.

As used herein, the term “bispecific antibody” refers to a multi-specific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv. or fragment thereof, having binding specificity for a first epitope, and a scFv, or fragment thereof, having binding specificity for a second epitope. In an embodiment, the first epitope is located on BTLA and the second epitope is located on PD-1, PD-L1, CTLA-4, EGFR, HER-2, CD19, CD20, CD33, CD3, and/or other tumor associated immune suppressors or surface antigens.

As used herein, an antibody that “specifically binds to BTLA” refers to an antibody and/or antigen binding domain that binds to BTLA, preferably human BTLA, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. In certain embodiments, the antibody and/or antigen-binding domain binds to cynomolgus BTLA. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen. In certain embodiments, the isolated monoclonal antibody or antigen-binding fragment thereof binds to human BTLA with a K_{D} of less than about 25 nM, 20 nM, 15 nM, 5 nM, 2 nM, 1 nM, or 0.5 nM.

As used herein the term “IC₅₀” refers to the half maximal inhibitory concentration of a monoclonal or bispecific antibody or antigen-binding fragment thereof of the invention. IC₅₀ is a measure of the potency of the monoclonal or bispecific antibody or antigen-binding fragment thereof of the invention for inhibiting the binding of HVEM or inhibiting the function of BTLA in a cell. In certain embodiments, the monoclonal antibody or antigen-binding fragment thereof or the bispecific antibody or antigen-binding fragment thereof has a KD of less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

As used herein the term “EC₅₀” refers to the half maximal effective concentration of a monoclonal or bispecific antibody or antigen-binding fragment thereof of the invention. EC₅₀ refers to the concentration of a monoclonal or bispecific antibody or antigen-binding fragment thereof for inducing a biological response (i.e., cell death) halfway between the baseline and maximum over a specified exposure time. In certain embodiments, the monoclonal antibody or antigen-binding fragment thereof or the bispecific antibody or antigen-binding fragment thereof has an EC₅₀ of less than about 1 μM, about 1000 nM to about 100 nM, about 100 nM to about 10 nM, about 10 nM to about 1 nM, about 1000 pM to about 500 pM, about 500 pM to about 200 pM, less than about 200 pM, about 200 pM to about 150 pM, about 200 pM to about 100 pM, about 100 pM to about 10 pM, or about 10 pM to about 1 pM.

According to a particular aspect, the invention relates to an isolated monoclonal antibody or antigen-binding fragment thereof, wherein the monoclonal antibody or antigen-binding fragment thereof or antigen binding domain comprises a heavy chain complementarity determining region 1 (HCDR1), a HCDR2, a HCDR3, a light chain complementarity determining region 1 (LCDR1), a LCDR2, and a LCDR3, having the polypeptide sequences of;
(1) SEQ ID NOS: 7, 8, 153, 10, 11, and 12, respectively: or
(2) SEQ ID NOs: 1, 2, 3, 4, 5, and 6, respectively:

wherein the antibody or antigen-binding fragment thereof or antigen binding domain thereof specifically binds B- and T-lymphocyte attenuator (BTLA), preferably human BTLA, optionally, said monoclonal antibody or antigen-binding fragment thereof is not naturally occurring. SEQ ID NO:153 is represented by the amino acid sequence CAREDGYPYYTLDX₁W, wherein X₁ is an amino acid selected from C, A, S, T, or V.

In certain embodiments, the isolated monoclonal antibodies or antigen-binding fragments thereof comprise a HCDR1, HCDR2, HCDR3, LCDR1, LCDR2, and LCDR3, having the polypeptide sequences of;
(1) SEQ ID NOs: 7, 8, 9, 10, 11, and 12, respectively; or
(2) SEQ ID NOs: 1, 2, 3, 4, 5, and 6, respectively;
   wherein the antibody or antigen-binding fragment thereof specifically binds B- and T-lymphocyte attenuator (BTLA), preferably human BTLA, optionally, said monoclonal antibody or antigen-binding fragment thereof is not naturally occurring.

According to another particular aspect, the invention relates to an isolated monoclonal antibody or antigen-binding fragment thereof, wherein the monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:15 or 13, or a light chain variable region having a polypeptide sequence at least 85%, preferably 90%, more preferably 95% or more, such as 95%, 96%, 97%, 98%, or 99% identical to SEQ ID NO:16 or 14. According to one preferred embodiment, the isolated monoclonal antibody or antigen-binding fragment thereof of the invention comprises a heavy chain variable region having the polypeptide sequence at least 85%, preferably 90%, more preferably 95% or more, such as 95%, 96%, 97%, 98%, or 99% identical to SEQ ID NO:13, and a light chain variable region having a polypeptide sequence at least 85%, preferably 90%, more preferably 95% or more, such as 95%, 96%, 97%, 98%, or 99% identical to SEQ ID NO:14, respectively. According to one preferred embodiment, the isolated monoclonal antibody or antigen-binding fragment thereof of the invention comprises a heavy chain variable region having the polypeptide sequence at least 85%, preferably 90%, more preferably 95% or more, such as 95%, 96%, 97%, 98%, or 99% identical to SEQ ID NO:15, and a light chain variable region having a polypeptide sequence at least 85%, preferably 90%, more preferably 95% or more, such as 95%, 96%, 97%, 98%, or 99% identical to SEQ ID NO:16, respectively.

According to a particular embodiment, the isolated anti-BTLA monoclonal antibody or antigen-binding fragment thereof comprises;
(1) a heavy chain variable region having the polypeptide sequence of SEQ ID NO:15, and a light chain variable region having the polypeptide sequence of SEQ ID NO:16; or
(2) a heavy chain variable region having the polypeptide sequence of SEQ ID NO:13, and a light chain variable region having the polypeptide sequence of SEQ ID NO:14.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 3, 4, 5, and 6, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:13, and a light chain variable region having a polypeptide sequence at least 85%, preferably 90%, more preferably 95% or more, such as 95%, 96%, 97%, 98%, or 99% identical to SEQ ID NO:14. Preferably, the isolated monoclonal antibody or antigen-binding fragment thereof comprises a heavy chain variable region having the polypeptide sequence of SEQ ID NO:13; and a light chain variable region having the polypeptide sequence of SEQ ID NO:14.

In one embodiment, the invention relates to an isolated monoclonal antibody or antigen-binding fragment thereof, comprising HCDR1, HCDR2, HCDR3, LCDR1, LCDR2, and LCDR3, having the polypeptide sequences of SEQ ID NOs: 7, 8, 9, 10, 11, and 12, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:15, and a light chain variable region having a polypeptide sequence at least 85%, preferably 90%, more preferably 95% or more, such as 95%, 96%, 97%, 98%, or 99% identical to SEQ ID NO:16. Preferably, the isolated monoclonal antibody or antigen-binding fragment thereof comprises a heavy chain variable region having the polypeptide sequence of SEQ ID NO:15; and a light chain variable region having the polypeptide sequence of SEQ ID NO:16.

According to another particular aspect, the invention relates to an isolated monoclonal antibody or antigen-binding fragment thereof or a bispecific antibody or antigen-binding fragment thereof of the invention, wherein the monoclonal or bispecific antibody or antigen-binding fragment thereof is chimeric.

According to another particular aspect, the invention relates to an isolated monoclonal antibody or antigen-binding fragment thereof or a bispecific antibody or antigen-binding fragment thereof of the invention, wherein the monoclonal or bispecific antibody or antigen-binding fragment thereof is human or humanized.

According to another particular aspect, the invention relates to a humanized monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of;
(1) SEQ ID NOs: 7, 8, 150, 10, 11, and 12, respectively;
(2) SEQ ID NOs: 1, 2, 3, 4, 5, and 6, respectively;
(3) SEQ ID NOs: 7, 8, 9, 10, 11, and 12, respectively;
(4) SEQ ID NOs: 7, 8, 149, 10, 11, and 12, respectively;
(5) SEQ ID NOs: 7, 8, 151, 10, 11, and 12, respectively: or
(6) SEQ ID NOS: 7, 8, 152, 10, 11, and 12, respectively.

According to another particular aspect, the invention relates to a humanized monoclonal antibody or antigen-binding fragment thereof, wherein the monoclonal antibody or antigen-binding fragment thereof comprises a heavy chain variable region having a polypeptide sequence at least 90%, at least 91%, at least 92%, at least 93%, at least 94%, at least 95%, at least 96%, at least 97%, at least 98%, or at least 99% identical to SEQ ID NO:141, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 143, 145, or 147, or a light chain variable region having a polypeptide sequence at least 90%, at least 91%, at least 92%, at least 93%, at least 94%, at least 95%, at least 96%, at least 97%, at least 98%, or at least 99% identical to SEQ ID NO:142, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 144, 146, or 148.

According to another particular aspect, the humanized anti-BTLA monoclonal antibody or antigen-binding fragment thereof comprises;
(1) a heavy chain variable region having the polypeptide sequence of SEQ ID NO:141, and a light chain variable region having the polypeptide sequence of SEQ ID NO:142;
(2) a heavy chain variable region having the polypeptide sequence of SEQ ID NO:17, and a light chain variable region having the polypeptide sequence of SEQ ID NO:18;
(3) a heavy chain variable region having the polypeptide sequence of SEQ ID NO:19, and a light chain variable region having the polypeptide sequence of SEQ ID NO:20;
(4) a heavy chain variable region having the polypeptide sequence of SEQ ID NO:21, and a light chain variable region having the polypeptide sequence of SEQ ID NO:22;
(5) a heavy chain variable region having the polypeptide sequence of SEQ ID NO:23, and a light chain variable region having the polypeptide sequence of SEQ ID NO:24;
(6) a heavy chain variable region having the polypeptide sequence of SEQ ID NO:25, and a light chain variable region having the polypeptide sequence of SEQ ID NO:26;
(7) a heavy chain variable region having the polypeptide sequence of SEQ ID NO:27, and a light chain variable region having the polypeptide sequence of SEQ ID NO:28;
(8) a heavy chain variable region having the polypeptide sequence of SEQ ID NO:29, and a light chain variable region having the polypeptide sequence of SEQ ID NO:30;
(9) a heavy chain variable region having the polypeptide sequence of SEQ ID NO:31, and a light chain variable region having the polypeptide sequence of SEQ ID NO:32;
(10) a heavy chain variable region having the polypeptide sequence of SEQ ID NO:33, and a light chain variable region having the polypeptide sequence of SEQ ID NO:34;
(11) a heavy chain variable region having the polypeptide sequence of SEQ ID NO:35, and a light chain variable region having the polypeptide sequence of SEQ ID NO:36;
(12) a heavy chain variable region having the polypeptide sequence of SEQ ID NO:37, and a light chain variable region having the polypeptide sequence of SEQ ID NO:38;
(13) a heavy chain variable region having the polypeptide sequence of SEQ ID NO:39, and a light chain variable region having the polypeptide sequence of SEQ ID NO:40;
(14) a heavy chain variable region having the polypeptide sequence of SEQ ID NO:41, and a light chain variable region having the polypeptide sequence of SEQ ID NO:42;
(15) a heavy chain variable region having the polypeptide sequence of SEQ ID NO:43, and a light chain variable region having the polypeptide sequence of SEQ ID NO:44;
(16) a heavy chain variable region having the polypeptide sequence of SEQ ID NO:45, and a light chain variable region having the polypeptide sequence of SEQ ID NO:46;
(17) a heavy chain variable region having the polypeptide sequence of SEQ ID NO:47, and a light chain variable region having the polypeptide sequence of SEQ ID NO:48;
(18) a heavy chain variable region having the polypeptide sequence of SEQ ID NO:49, and a light chain variable region having the polypeptide sequence of SEQ ID NO:50;
(19) a heavy chain variable region having the polypeptide sequence of SEQ ID NO:51, and a light chain variable region having the polypeptide sequence of SEQ ID NO:52;
(20) a heavy chain variable region having the polypeptide sequence of SEQ ID NO:53, and a light chain variable region having the polypeptide sequence of SEQ ID NO:54;
(21) a heavy chain variable region having the polypeptide sequence of SEQ ID NO:55, and a light chain variable region having the polypeptide sequence of SEQ ID NO:56;
(22) a heavy chain variable region having the polypeptide sequence of SEQ ID NO:57, and a light chain variable region having the polypeptide sequence of SEQ ID NO:58;
(23) a heavy chain variable region having the polypeptide sequence of SEQ ID NO:59, and a light chain variable region having the polypeptide sequence of SEQ ID NO:60;
(24) a heavy chain variable region having the polypeptide sequence of SEQ ID NO:61, and a light chain variable region having the polypeptide sequence of SEQ ID NO:62;
(25) a heavy chain variable region having the polypeptide sequence of SEQ ID NO:63, and a light chain variable region having the polypeptide sequence of SEQ ID NO:64;
(26) a heavy chain variable region having the polypeptide sequence of SEQ ID NO:65, and a light chain variable region having the polypeptide sequence of SEQ ID NO:66;
(27) a heavy chain variable region having the polypeptide sequence of SEQ ID NO:67, and a light chain variable region having the polypeptide sequence of SEQ ID NO:68;
(28) a heavy chain variable region having the polypeptide sequence of SEQ ID NO:69, and a light chain variable region having the polypeptide sequence of SEQ ID NO:70;
(29) a heavy chain variable region having the polypeptide sequence of SEQ ID NO:71, and a light chain variable region having the polypeptide sequence of SEQ ID NO:72;
(30) a heavy chain variable region having the polypeptide sequence of SEQ ID NO:73, and a light chain variable region having the polypeptide sequence of SEQ ID NO:74;
(31) a heavy chain variable region having the polypeptide sequence of SEQ ID NO:75, and a light chain variable region having the polypeptide sequence of SEQ ID NO:76;
(32) a heavy chain variable region having the polypeptide sequence of SEQ ID NO:77, and a light chain variable region having the polypeptide sequence of SEQ ID NO:78;
(33) a heavy chain variable region having the polypeptide sequence of SEQ ID NO:79, and a light chain variable region having the polypeptide sequence of SEQ ID NO:80;
(34) a heavy chain variable region having the polypeptide sequence of SEQ ID NO:81, and a light chain variable region having the polypeptide sequence of SEQ ID NO:82;
(35) a heavy chain variable region having the polypeptide sequence of SEQ ID NO:83, and a light chain variable region having the polypeptide sequence of SEQ ID NO:84;
(36) a heavy chain variable region having the polypeptide sequence of SEQ ID NO:85, and a light chain variable region having the polypeptide sequence of SEQ ID NO:86;
(37) a heavy chain variable region having the polypeptide sequence of SEQ ID NO:87, and a light chain variable region having the polypeptide sequence of SEQ ID NO:88;
(38) a heavy chain variable region having the polypeptide sequence of SEQ ID NO:89, and a light chain variable region having the polypeptide sequence of SEQ ID NO:90;
(39) a heavy chain variable region having the polypeptide sequence of SEQ ID NO:91, and a light chain variable region having the polypeptide sequence of SEQ ID NO:92;
(40) a heavy chain variable region having the polypeptide sequence of SEQ ID NO:93, and a light chain variable region having the polypeptide sequence of SEQ ID NO:94;
(41) a heavy chain variable region having the polypeptide sequence of SEQ ID NO:95, and a light chain variable region having the polypeptide sequence of SEQ ID NO:96;
(42) a heavy chain variable region having the polypeptide sequence of SEQ ID NO:97, and a light chain variable region having the polypeptide sequence of SEQ ID NO:98;
(43) a heavy chain variable region having the polypeptide sequence of SEQ ID NO:99, and a light chain variable region having the polypeptide sequence of SEQ ID NO:100;
(44) a heavy chain variable region having the polypeptide sequence of SEQ ID NO:101, and a light chain variable region having the polypeptide sequence of SEQ ID NO:102;
(45) a heavy chain variable region having the polypeptide sequence of SEQ ID NO:103, and a light chain variable region having the polypeptide sequence of SEQ ID NO:104;
(46) a heavy chain variable region having the polypeptide sequence of SEQ ID NO:105, and a light chain variable region having the polypeptide sequence of SEQ ID NO:106;
(47) a heavy chain variable region having the polypeptide sequence of SEQ ID NO:107, and a light chain variable region having the polypeptide sequence of SEQ ID NO:108;
(48) a heavy chain variable region having the polypeptide sequence of SEQ ID NO:109, and a light chain variable region having the polypeptide sequence of SEQ ID NO:110;
(49) a heavy chain variable region having the polypeptide sequence of SEQ ID NO:111, and a light chain variable region having the polypeptide sequence of SEQ ID NO:112;
(50)) a heavy chain variable region having the polypeptide sequence of SEQ ID NO:113, and a light chain variable region having the polypeptide sequence of SEQ ID NO:114;
(51) a heavy chain variable region having the polypeptide sequence of SEQ ID NO:115, and a light chain variable region having the polypeptide sequence of SEQ ID NO:116;
(52) a heavy chain variable region having the polypeptide sequence of SEQ ID NO:117, and a light chain variable region having the polypeptide sequence of SEQ ID NO:118;
(53) a heavy chain variable region having the polypeptide sequence of SEQ ID NO:119, and a light chain variable region having the polypeptide sequence of SEQ ID NO:120;
(54) a heavy chain variable region having the polypeptide sequence of SEQ ID NO:121, and a light chain variable region having the polypeptide sequence of SEQ ID NO:122;
(55) a heavy chain variable region having the polypeptide sequence of SEQ ID NO:123, and a light chain variable region having the polypeptide sequence of SEQ ID NO:124;
(56) a heavy chain variable region having the polypeptide sequence of SEQ ID NO:125, and a light chain variable region having the polypeptide sequence of SEQ ID NO:126;
(57) a heavy chain variable region having the polypeptide sequence of SEQ ID NO:127, and a light chain variable region having the polypeptide sequence of SEQ ID NO:128;
(58) a heavy chain variable region having the polypeptide sequence of SEQ ID NO:129, and a light chain variable region having the polypeptide sequence of SEQ ID NO:130;
(59) a heavy chain variable region having the polypeptide sequence of SEQ ID NO:131, and a light chain variable region having the polypeptide sequence of SEQ ID NO:132;
(60) a heavy chain variable region having the polypeptide sequence of SEQ ID NO:133, and a light chain variable region having the polypeptide sequence of SEQ ID NO:134;
(61) a heavy chain variable region having the polypeptide sequence of SEQ ID NO:135, and a light chain variable region having the polypeptide sequence of SEQ ID NO:136;
(62) a heavy chain variable region having the polypeptide sequence of SEQ ID NO:137, and a light chain variable region having the polypeptide sequence of SEQ ID NO:138;
(63) a heavy chain variable region having the polypeptide sequence of SEQ ID NO:139, and a light chain variable region having the polypeptide sequence of SEQ ID NO:140;
(64) a heavy chain variable region having the polypeptide sequence of SEQ ID NO:143, and a light chain variable region having the polypeptide sequence of SEQ ID NO:144;
(65) a heavy chain variable region having the polypeptide sequence of SEQ ID NO:145, and a light chain variable region having the polypeptide sequence of SEQ ID NO:146; or
(66) a heavy chain variable region having the polypeptide sequence of SEQ ID NO:147, and a light chain variable region having the polypeptide sequence of SEQ ID NO:148.

In one embodiment, the invention relates to an isolated humanized monoclonal antibody or antigen-binding fragment thereof, comprising HCDR1, HCDR2, HCDR3, LCDR1, LCDR2, and LCDR3, having the polypeptide sequences of SEQ ID NOs: 1, 2, 3, 4, 5, and 6, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 17, 19, 21, 23, 25, 27, 29, 31, 33, 35, 37, 39, 41, 43, 45, 47, 49, 51, 53, 55, or 57, respectively, and a light chain variable region having a polypeptide sequence at least 85%, preferably 90%, more preferably 95% or more, such as 95%, 96%, 97%, 98%, or 99% identical to SEQ ID NO: 18, 20, 22, 24, 26, 28, 30, 32, 30) 34, 36, 38, 40, 42, 44, 46, 48, 50, 52, 54, 56, or 58, respectively. Preferably, the isolated humanized monoclonal antibody or antigen-binding fragment thereof comprises a heavy chain variable region having the polypeptide sequence of SEQ ID NO: 17, 19, 21, 23, 25, 27. 29, 31, 33, 35, 37, 39, 41, 43, 45, 47, 49, 51, 53, 55, or 57, respectively; and a light chain variable region having the polypeptide sequence of SEQ ID NO: 18, 20, 22, 24, 26, 28, 30, 32, 34, 36, 38, 40, 42, 44, 46, 48, 50, 52, 54, 56, or 58, respectively.

In one embodiment, the invention relates to an isolated humanized monoclonal antibody or antigen-binding fragment thereof, comprising HCDR1, HCDR2, HCDR3, LCDR1, LCDR2, and LCDR3, having the polypeptide sequences of SEQ ID NOs: 7, 8, 9, 10, 11, and 12, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO: 59, 61, 63, 65, 67, 69, 71, 73, 75, 77, 79, 81, 83, 85, 87, 89, 91, 93, 95, 97, or 99, respectively, and a light chain variable region having a polypeptide sequence at least 85%, preferably 90%, more preferably 95% or more, such as 95%, 96%, 97%, 98%, or 99% identical to SEQ ID NO: 60, 62, 64, 66, 68, 70, 72, 74, 76, 78, 80, 82, 84, 86, 88, 90, 92, 94, 96, 98, or 100, respectively. Preferably, the isolated humanized monoclonal antibody or antigen-binding fragment thereof comprises a heavy chain variable region having the polypeptide sequence of SEQ ID NO: 59, 61, 63, 65, 67, 69. 71, 73, 75, 77, 79, 81, 83, 85, 87, 89, 91, 93, 95, 97, or 99, respectively; and a light chain variable region having the polypeptide sequence of SEQ ID NO: 60, 62, 64, 66, 68, 70, 72. 74, 76, 78, 80, 82, 84, 86, 88, 90, 92, 94, 96, 98, or 100, respectively.

In one embodiment, the invention relates to an isolated humanized monoclonal antibody or antigen-binding fragment thereof, comprising HCDR1, HCDR2, HCDR3, LCDR1, LCDR2, and LCDR3, having the polypeptide sequences of SEQ ID NOs: 7, 8, 149, 10, 11, and 12, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:101, 109, 117, 125, 127, 129, 131. 133, or 135, respectively, and a light chain variable region having a polypeptide sequence at least 85%, preferably 90%, more preferably 95% or more, such as 95%, 96%, 97%, 98%, or 99% identical to SEQ ID NO:102, 110, 118, 126, 128, 130, 132, 134, or 136, respectively. Preferably, the isolated humanized monoclonal antibody or antigen-binding fragment thereof comprises a heavy chain variable region having the polypeptide sequence of SEQ ID NO:101, 109, 117, 125, 127, 129, 131, 133, or 135, respectively; and a light chain variable region having the polypeptide sequence of SEQ ID NO:102, 110, 118, 126, 128, 130, 132. 134, or 136, respectively.

In one embodiment, the invention relates to an isolated humanized monoclonal antibody or antigen-binding fragment thereof, comprising HCDR1, HCDR2, HCDR3, LCDR1, LCDR2, and LCDR3, having the polypeptide sequences of SEQ ID NOs: 7, 8, 150, 10, 11, and 12, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:103, 111, 119, 137, 139, 141, 143. 145, or 147, respectively, and a light chain variable region having a polypeptide sequence at least 85%, preferably 90%, more preferably 95% or more, such as 95%, 96%, 97%, 98%, or 99% identical to SEQ ID NO:104, 112, 120, 138, 140, 142, 144, 146, or 148, respectively. Preferably, the isolated humanized monoclonal antibody or antigen-binding fragment thereof comprises a heavy chain variable region having the polypeptide sequence of SEQ ID NO:103, 111, 119, 137, 139, 141, 143, 145, or 147, respectively; and a light chain variable region having the polypeptide sequence of SEQ ID NO:104, 112, 120, 138, 140, 142, 144, 146, or 148, respectively.

In one embodiment, the invention relates to an isolated humanized monoclonal antibody or antigen-binding fragment thereof, comprising HCDR1, HCDR2, HCDR3, LCDR1, LCDR2, and LCDR3, having the polypeptide sequences of SEQ ID NOs: 7, 8, 151, 10, 11, and 12, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:105, 113, or 121, respectively, and a light chain variable region having a polypeptide sequence at least 85%, preferably 90%, more preferably 95% or more, such as 95%, 96%, 97%, 98%, or 99% identical to SEQ ID NO:106, 114, or 122, respectively. Preferably, the isolated humanized monoclonal antibody or antigen-binding fragment thereof comprises a heavy chain variable region having the polypeptide sequence of SEQ ID NO:105, 113, or 121, respectively; and a light chain variable region having the polypeptide sequence of SEQ ID NO:106, 114, or 122, respectively.

In one embodiment, the invention relates to an isolated humanized monoclonal antibody or antigen-binding fragment thereof, comprising HCDR1, HCDR2, HCDR3, LCDR1, LCDR2, and LCDR3, having the polypeptide sequences of SEQ ID NOs: 7, 8, 152, 10, 11, and 12, respectively. In another embodiment, the isolated humanized monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:107, 115, or 123, respectively, and a light chain variable region having a polypeptide sequence at least 85%, preferably 90%, more preferably 95% or more, such as 95%, 96%, 97%, 98%, or 99% identical to SEQ ID NO:108, 116, or 124, respectively. Preferably, the isolated humanized monoclonal antibody or antigen-binding fragment thereof comprises a heavy chain variable region having the polypeptide sequence of SEQ ID NO:107, 115, or 123, respectively; and a light chain variable region having the polypeptide sequence of SEQ ID NO:108, 116, or 124, respectively.

In certain embodiments, the isolated monoclonal antibody or antigen-binding fragment thereof is an agonist of human BTLA and activates downstream signaling from BTLA upon binding to BTLA.

In certain embodiments, the monoclonal antibody or antigen-binding fragment thereof is capable of inhibiting B cell proliferation and/or T cell and/or plasma cell activation. The T-cell can, for example, be selected from the group consisting of a CD4 T-cell, a CD8 T-cell, a Th1 T-cell, a TFH T-cell, an αβ T-cell, and a γδ T-cell.

In certain embodiments, the monoclonal antibody or antigen-binding fragment thereof is capable of blocking or abolishing BTLA binding to herpes virus entry mediator (HVEM).

In certain embodiments, the isolated monoclonal antibody or antigen-binding fragment thereof binds to BTLA and is capable of increasing pro-inflammatory cytokine production, such as, for example, interferon γ (IFNγ), interleukin-2 (IL-2), C—X—C motif chemokine ligand 9 (CXCL9), C—X—C motif chemokine ligand 10 (CXCL10), interleukin 1ß (IL1β), and tumor necrosis factor α (TNFα). In certain embodiments, the isolated monoclonal antibody or antigen-binding fragment thereof is capable of increasing interferon γ (IFNγ), interleukin-2 (IL-2), C—X—C motif chemokine ligand 9 (CXCL9), C—X—C motif chemokine ligand 10 (CXCL10), interleukin 1β (IL1β), and/or tumor necrosis factor α (TNFα) production more than a control anti-BTLA antibody or antigen-binding fragment thereof. As used herein a control anti-BTLA antibody can be an antibody capable of binding BTLA and blocking binding of BTLA to HVEM. An example of a control antibody used herein can include, but is not limited to, the JS004 anti-BTLA antibody.

In another general aspect, the invention relates to an isolated nucleic acid encoding a monoclonal antibody or antigen-binding fragment thereof and/or a bispecific antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies or antigen-binding fragments thereof of the invention can be altered without changing the amino acid sequences of the proteins.

In another general aspect, the invention relates to a vector comprising an isolated nucleic acid encoding a monoclonal antibody or antigen-binding fragment thereof and/or a bispecific antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated nucleic acid encoding a monoclonal antibody or antigen-binding fragment thereof and/or a bispecific antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a monoclonal antibody or antigen-binding fragment thereof and/or a bispecific antibody or antigen-binding fragment thereof of the invention, comprising culturing a cell comprising a nucleic acid encoding the monoclonal antibody or antigen-binding fragment thereof or bispecific antibody or antigen-binding fragment thereof under conditions to produce a monoclonal antibody or antigen-binding fragment thereof or bispecific antibody or antigen-binding fragment thereof of the invention, and recovering the monoclonal and/or bispecific antibody or antigen-binding fragment thereof from the cell or cell culture (e.g., from the supernatant). Expressed monoclonal and/or bispecific antibodies or antigen-binding fragments thereof can be harvested from the cells and purified according to conventional techniques known in the art and as described herein.

### Pharmaceutical Compositions

In another general aspect, the invention relates to a pharmaceutical composition, comprising an isolated monoclonal antibody or antigen-binding fragment thereof, a bispecific antibody or antigen-binding fragment thereof, an isolated polynucleotide, and/or an isolated polypeptide of the invention and a pharmaceutically acceptable carrier.

The term “pharmaceutical composition” as used herein means a product comprising an isolated polynucleotide of the invention, an isolated polypeptide of the invention, an anti-BTLA monoclonal antibody or antigen-binding fragment thereof, and/or a bispecific antibody of the invention together with a pharmaceutically acceptable carrier. Polynucleotides, polypeptides, an anti-BTLA monoclonal antibody or antigen-binding fragment thereof, and/or a bispecific antibody of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g., 21^{st} edition (2005), and any later editions). Non-limiting examples of additional ingredients include buffers, diluents, solvents, tonicity regulating agents, preservatives, stabilizers, and chelating agents. One or more pharmaceutically acceptable carrier(s) can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms may be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris (hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxy benzoate, chlorobutanol, chlorocresol, chlorohexidine, chlorphenesin, o-cresol, m-cresol, p-cresol, ethyl 4-hydroxy benzoate, imidurea, methyl 4-hydroxy benzoate, phenol, 2-phenoxyethanol, 2-phenylethanol, propyl 4-hydroxy benzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g., PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar 30) alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivatives thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a monoclonal antibody or antigen-binding fragment thereof and/or a bispecific antibody or antigen-binding fragment thereof of the invention, comprising combining a monoclonal antibody or antigen-binding fragment thereof and/or a bispecific antibody or antigen-binding fragment thereof with a pharmaceutically acceptable carrier to obtain the pharmaceutical composition.

### Methods of Use

In another general aspect, the invention relates to a method of treating a cancer in a subject in need thereof, comprising administering to the subject the pharmaceutical compositions comprising anti-BTLA monoclonal and/or bispecific antibodies or antigen binding fragments thereof of the invention. In certain embodiments, the cancer is a solid tumor, preferably a solid tumor with infiltrating T cells, more preferably a solid tumor with infiltrating T effector cells, more preferably a solid tumor with T effector cells expressing BTLA, most preferably a solid tumor with infiltrating T effector cells expressing BTLA and BTLA ligand HVEM highly expressing in the tumor microenvironment. The cancer can, for example, be selected from, but not limited to, melanoma, lung cancer, renal cell carcinoma, and liver cancer.

In another general aspect, the invention relates to a method of targeting BTLA on a cancer cell surface in a subject to achieve cell killing, the method comprising administering to the subject an isolated monoclonal antibody or antigen binding fragment thereof and/or bispecific antibody or antigen-binding fragment thereof that specifically binds BTLA or a pharmaceutical composition comprising the isolated monoclonal antibody or antigen binding fragment thereof and/or bispecific antibody or antigen-binding fragment thereof of the invention. Binding of the anti-BTLA monoclonal or bispecific antibody or antigen-binding fragment to BTLA can block binding of herpes virus entry mediator (HVEM) to BTLA; activate downstream signaling from BTLA upon binding to BTLA; can inhibit B-cell proliferation and/or T-cell and/or plasma cell activation; increase pro-inflammatory cytokine production, such as, for example, IL-2, CXCL9, CXCL10, IL1β, and TNFα. The monoclonal or bispecific antibody or antigen binding fragment thereof can, for example, serve to recruit conjugated drugs, and/or can form a bispecific antibody with another monoclonal antibody to mediate the death of the targeted cancer cell.

Binding of the anti-BTLA monoclonal or bispecific antibody or antigen-binding fragment to BTLA can increase pro-inflammatory cytokine production, such as, for example, IL-2, CXCL9, CXCL10, IL1β, and TNFα more than a control anti-BTLA monoclonal or bispecific antibody or antigen-binding fragment thereof. As used herein a control anti-BTLA antibody can be an antibody capable of binding BTLA and blocking binding of BTLA to HVEM. An example of a control antibody used herein can include, but is not limited to, the JS004 anti-BTLA antibody.

The functional activity of antibodies and antigen-binding fragments thereof that bind BTLA can be characterized by methods known in the art and as described herein. Methods for characterizing antibodies and antigen-binding fragments thereof that bind BTLA include, but are not limited to, affinity and specificity assays including Biacore, ELISA, and OctetRed analysis, and detection of the binding of antibodies and antigen-binding fragments to BTLA on cells (either cells transfected with BTLA or cells that naturally express BTLA) by FACS. According to particular embodiments, the methods for characterizing antibodies and antigen-binding fragments thereof that bind BTLA include those described below.

In another general aspect, the invention relates to a method of treating a cancer in a subject in need thereof, comprising administering to the subject an isolated monoclonal antibody or antigen-binding fragment thereof and/or bispecific antibody or antigen-binding fragment thereof that specifically binds BTLA or a pharmaceutical composition of the invention. The cancer can, for example, be selected from the group consisting of melanoma, lung cancer, renal cell carcinoma, and liver cancer.

As used herein with reference to anti-BTLA antibodies or antigen-binding fragments thereof, a therapeutically effective amount means an amount of the anti-BTLA antibody or antigen-binding fragment thereof that modulates an immune response in a subject in need thereof. Also, as used herein with reference to anti-BTLA antibodies or antigen-binding fragments thereof, a therapeutically effective amount means an amount of the anti-BTLA antibody or antigen-binding fragment thereof that results in treatment of a disease, disorder, or condition: prevents or slows the progression of the disease, disorder, or condition: or reduces or completely alleviates symptoms associated with the disease, disorder, or condition.

According to particular embodiments, the disease, disorder or condition to be treated is cancer. The cancer can, for example, be melanoma, lung cancer, renal cell carcinoma, and liver cancer.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith: (ii) reduce the duration of the disease, disorder or condition to be treated, or a symptom associated therewith: (iii) prevent the progression of the disease, disorder or condition to be treated, or a symptom associated therewith: (iv) cause regression of the disease, disorder or condition to be treated, or a symptom associated therewith: (v) prevent the development or onset of the disease, disorder or condition to be treated, or a symptom associated therewith: (vi) prevent the recurrence of the disease, disorder or condition to be treated, or a symptom associated therewith: (vii) reduce hospitalization of a subject having the disease, disorder or condition to be treated, or a symptom associated therewith: (viii) reduce hospitalization length of a subject having the disease, disorder or condition to be treated, or a symptom associated therewith: (ix) increase the survival of a subject with the disease, disorder or condition to be treated, or a symptom associated therewith: (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the provided compositions can be used in combination with another treatment including, but not limited to, a chemotherapy, an anti-CD20 mAb, an anti-EGFR mAb, an anti-HER-2 mAb, an anti-CD19 mAb, an anti-CD33 mAb, an anti-CD47 mAb, an anti-CD73 mAb, an anti-PD-1 mAb, an anti-PD-L1 mAb, an anti-CTLA mAb, an anti-TNFR2 mAb, an anti-OX40) mAb, other immuno-oncology drugs, an antiangiogenic agent, a radiation therapy, an antibody-drug conjugate (ADC), a targeted therapy, other anticancer drugs, and/or treatments targeting immunomodulatory targets including, but not limited to PD1 and PD-LI. Antibodies against BTLA can be used to construct bispecific antibodies with partner mAbs against PD-1, PD-L1, CTLA-4, CTLA, TNFR2, OX40, EGFR, HER-2, CD19, CD20, CD33, CD73, CD47, and/or CD3. Two antibodies that recognize two different epitopes on BTLA can also be used to construct a bispecific antibody to treat cancers/tumors that express BTLA.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In another general aspect, the invention relates to a method of determining a level of a BTLA in a subject. The methods comprise (a) obtaining a sample from the subject: (b) contacting the sample with a monoclonal antibody or antigen-binding fragment thereof of the invention; and (c) determining a level of BTLA in the subject.

As used herein, “sample” refers to a biological sample isolated from a subject and can include, but is not limited to, whole blood, serum, plasma, blood cells, endothelial cells, tissue biopsies (e.g., a cancer tissue), lymphatic fluid, ascites fluid, interstitial fluid, bone marrow; cerebrospinal fluid, saliva, mucous, sputum, sweat, urine, or any other secretion, excretion, or other bodily fluids. A “blood sample” refers to whole blood or any fraction thereof, including blood cells, serum, and plasma. A sample can, for example, comprise Treg cells.

In certain embodiments, the level of BTLA in the subject can be determined utilizing assays selected from, but not limited to, a Western blot assay, immunohistochemistry (IHC) and an ELISA. Relative protein levels can be determined by utilizing Western blot analysis and IHC, and absolute protein levels can be determined by utilizing an ELISA. When determining the relative levels of BTLA, the levels of BTLA can be determined between at least two samples, e.g., between samples from the same subject at different time points, between samples from different tissues in the same subject, and/or between samples from different subjects. Alternatively, when determining absolute levels of BTLA, such as by an ELISA, the absolute level of BTLA in the sample can be determined by creating a standard for the ELISA prior to testing the sample. A person skilled in the art would understand which analytical techniques to utilize to determine the level of BTLA in a sample from the subject utilizing the antibodies or antigen-binding fragments thereof of the invention.

Utilizing methods of determining a level of BTLA in a sample from a subject can lead to the diagnosis of abnormal (elevated, reduced, or insufficient) BTLA levels in a disease and making appropriate therapeutic decisions. Such a disease can be a cancer. Additionally, by monitoring the levels of BTLA in a subject, the risk of developing a disease as indicated above can be determined based on the knowledge of the level of BTLA in a particular disease and/or during the progression of the particular disease.

### EMBODIMENTS

The invention provides also the following non-limiting embodiments.

Embodiment 1 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of;
(1) SEQ ID NOs: 7, 8, 153, 10, 11, and 12, respectively: or
(2) SEQ ID NOs: 1, 2, 3, 4, 5, and 6, respectively:
   wherein the antibody or antigen-binding fragment thereof specifically binds BTLA, preferably specifically binds human BTLA, optionally, said monoclonal antibody or antigen-binding fragment thereof is not naturally occurring.

Embodiment 2 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of;
(1) SEQ ID NOs: 7, 8, 9, 10, 11, and 12, respectively: or
(2) SEQ ID NOs: 1, 2, 3, 4, 5, and 6, respectively:
   wherein the antibody or antigen-binding fragment thereof specifically binds BTLA, preferably specifically binds human BTLA, optionally, said monoclonal antibody or antigen-binding fragment thereof is not naturally occurring.

Embodiment 3 is the isolated monoclonal antibody or antigen-binding fragment thereof of embodiment 1 or 2, comprising a heavy chain variable region having a polypeptide sequence at least 95% identical to SEQ ID NO:15 or 13, or a light chain variable region having a polypeptide sequence at least 95% identical to SEQ ID NO:16 or 14.

Embodiment 4 is the isolated monoclonal antibody or antigen-binding fragment thereof of any one of embodiments 1-3, comprising;
(1) a heavy chain variable region having the polypeptide sequence of SEQ ID NO:15, and a light chain variable region having the polypeptide sequence of SEQ ID NO:16; or
(2) a heavy chain variable region having the polypeptide sequence of SEQ ID NO:13, and a light chain variable region having the polypeptide sequence of SEQ ID NO:14.

Embodiment 5 is the isolated monoclonal antibody or antigen-binding fragment thereof of any one of embodiments 1-4, wherein the antibody or antigen-binding fragment thereof is chimeric and/or human or humanized.

Embodiment 6 is the isolated humanized monoclonal antibody or antigen-binding fragment thereof of embodiment 5, wherein the isolated monoclonal antibody or antigen-binding fragment thereof comprises a heavy chain complementarity determining region 1 (HCDR1), HCDR2, HCDR3, a light chain complementarity determining region 1 (LCDR1), LCDR2, and LCDR3, having the polypeptide sequences of;
(1) SEQ ID NOs: 7, 8, 150, 10, 11, and 12, respectively;
(2) SEQ ID NOS: 1, 2, 3, 4, 5, and 6, respectively;
(3) SEQ ID NOs: 7, 8, 9, 10, 11, and 12, respectively;
(4) SEQ ID NOs: 7, 8, 149, 10, 11, and 12, respectively;
(5) SEQ ID NOs: 7, 8, 151, 10, 11, and 12, respectively: or
(6) SEQ ID NOs: 7, 8, 152, 10, 11, and 12, respectively.

Embodiment 7 is the isolated humanized monoclonal antibody or antigen-binding fragment thereof of embodiment 6, wherein the isolated monoclonal antibody or antigen-binding fragment thereof comprises a heavy chain variable region having a polypeptide sequence at least 95% identical to SEQ ID NO:141, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 143, 145, or 147, or a light chain variable region having a polypeptide sequence at least 95% identical to SEQ ID NO:142, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 144, 146, or 148.

Embodiment 8 is the isolated humanized monoclonal antibody or antigen-binding fragment thereof of embodiment 7, wherein the isolated monoclonal antibody or antigen-binding fragment thereof comprises;
(1) a heavy chain variable region having the polypeptide sequence of SEQ ID NO:141, and a light chain variable region having the polypeptide sequence of SEQ ID NO:142;
(2) a heavy chain variable region having the polypeptide sequence of SEQ ID NO:17, and a light chain variable region having the polypeptide sequence of SEQ ID NO:18;
(3) a heavy chain variable region having the polypeptide sequence of SEQ ID NO:19, and a light chain variable region having the polypeptide sequence of SEQ ID NO:20;
(4) a heavy chain variable region having the polypeptide sequence of SEQ ID NO:21, and a light chain variable region having the polypeptide sequence of SEQ ID NO:22;
(5) a heavy chain variable region having the polypeptide sequence of SEQ ID NO:23, and a light chain variable region having the polypeptide sequence of SEQ ID NO:24;
(6) a heavy chain variable region having the polypeptide sequence of SEQ ID NO:25, and a light chain variable region having the polypeptide sequence of SEQ ID NO:26;
(7) a heavy chain variable region having the polypeptide sequence of SEQ ID NO:27, and a light chain variable region having the polypeptide sequence of SEQ ID NO:28;
(8) a heavy chain variable region having the polypeptide sequence of SEQ ID NO:29, and a light chain variable region having the polypeptide sequence of SEQ ID NO:30;
(9) a heavy chain variable region having the polypeptide sequence of SEQ ID NO:31, and a light chain variable region having the polypeptide sequence of SEQ ID NO:32;
(10) a heavy chain variable region having the polypeptide sequence of SEQ ID NO:33, and a light chain variable region having the polypeptide sequence of SEQ ID NO:34;
(11) a heavy chain variable region having the polypeptide sequence of SEQ ID NO:35, and a light chain variable region having the polypeptide sequence of SEQ ID NO:36;
(12) a heavy chain variable region having the polypeptide sequence of SEQ ID NO:37, and a light chain variable region having the polypeptide sequence of SEQ ID NO:38;
(13) a heavy chain variable region having the polypeptide sequence of SEQ ID NO:39, and a light chain variable region having the polypeptide sequence of SEQ ID NO:40;
(14) a heavy chain variable region having the polypeptide sequence of SEQ ID NO:41, and a light chain variable region having the polypeptide sequence of SEQ ID NO:42;
(15) a heavy chain variable region having the polypeptide sequence of SEQ ID NO:43, and a light chain variable region having the polypeptide sequence of SEQ ID NO:44;
(16) a heavy chain variable region having the polypeptide sequence of SEQ ID NO:45, and a light chain variable region having the polypeptide sequence of SEQ ID NO:46;
(17) a heavy chain variable region having the polypeptide sequence of SEQ ID NO:47, and a light chain variable region having the polypeptide sequence of SEQ ID NO:48;
(18) a heavy chain variable region having the polypeptide sequence of SEQ ID NO:49, and a light chain variable region having the polypeptide sequence of SEQ ID NO:50;
(19) a heavy chain variable region having the polypeptide sequence of SEQ ID NO:51, and a light chain variable region having the polypeptide sequence of SEQ ID NO:52;
(20) a heavy chain variable region having the polypeptide sequence of SEQ ID NO:53, and a light chain variable region having the polypeptide sequence of SEQ ID NO:54;
(21) a heavy chain variable region having the polypeptide sequence of SEQ ID NO:55, and a light chain variable region having the polypeptide sequence of SEQ ID NO:56;
(22) a heavy chain variable region having the polypeptide sequence of SEQ ID NO:57, and a light chain variable region having the polypeptide sequence of SEQ ID NO:58;
(23) a heavy chain variable region having the polypeptide sequence of SEQ ID NO:59, and a light chain variable region having the polypeptide sequence of SEQ ID NO:60;
(24) a heavy chain variable region having the polypeptide sequence of SEQ ID NO:61, and a light chain variable region having the polypeptide sequence of SEQ ID NO:62;
(25) a heavy chain variable region having the polypeptide sequence of SEQ ID NO:63, and a light chain variable region having the polypeptide sequence of SEQ ID NO:64;
(26) a heavy chain variable region having the polypeptide sequence of SEQ ID NO:65, and a light chain variable region having the polypeptide sequence of SEQ ID NO:66;
(27) a heavy chain variable region having the polypeptide sequence of SEQ ID NO:67, and a light chain variable region having the polypeptide sequence of SEQ ID NO:68;
(28) a heavy chain variable region having the polypeptide sequence of SEQ ID NO:69, and a light chain variable region having the polypeptide sequence of SEQ ID NO:70;
(29) a heavy chain variable region having the polypeptide sequence of SEQ ID NO:71, and a light chain variable region having the polypeptide sequence of SEQ ID NO:72;
(30) a heavy chain variable region having the polypeptide sequence of SEQ ID NO:73, and a light chain variable region having the polypeptide sequence of SEQ ID NO:74;
(31) a heavy chain variable region having the polypeptide sequence of SEQ ID NO:75, and a light chain variable region having the polypeptide sequence of SEQ ID NO:76;
(32) a heavy chain variable region having the polypeptide sequence of SEQ ID NO:77, and a light chain variable region having the polypeptide sequence of SEQ ID NO:78;
(33) a heavy chain variable region having the polypeptide sequence of SEQ ID NO:79, and a light chain variable region having the polypeptide sequence of SEQ ID NO:80;
(34) a heavy chain variable region having the polypeptide sequence of SEQ ID NO:81, and a light chain variable region having the polypeptide sequence of SEQ ID NO:82;
(35) a heavy chain variable region having the polypeptide sequence of SEQ ID NO:83, and a light chain variable region having the polypeptide sequence of SEQ ID NO:84;
(36) a heavy chain variable region having the polypeptide sequence of SEQ ID NO:85, and a light chain variable region having the polypeptide sequence of SEQ ID NO:86;
(37) a heavy chain variable region having the polypeptide sequence of SEQ ID NO:87, and a light chain variable region having the polypeptide sequence of SEQ ID NO:88;
(38) a heavy chain variable region having the polypeptide sequence of SEQ ID NO:89, and a light chain variable region having the polypeptide sequence of SEQ ID NO:90;
(39) a heavy chain variable region having the polypeptide sequence of SEQ ID NO:91, and a light chain variable region having the polypeptide sequence of SEQ ID NO:92;
(40) a heavy chain variable region having the polypeptide sequence of SEQ ID NO:93, and a light chain variable region having the polypeptide sequence of SEQ ID NO:94;
(41) a heavy chain variable region having the polypeptide sequence of SEQ ID NO:95, and a light chain variable region having the polypeptide sequence of SEQ ID NO:96;
(42) a heavy chain variable region having the polypeptide sequence of SEQ ID NO:97, and a light chain variable region having the polypeptide sequence of SEQ ID NO:98;
(43) a heavy chain variable region having the polypeptide sequence of SEQ ID NO:99, and a light chain variable region having the polypeptide sequence of SEQ ID NO:100;
(44) a heavy chain variable region having the polypeptide sequence of SEQ ID NO:101, and a light chain variable region having the polypeptide sequence of SEQ ID NO:102;
(45) a heavy chain variable region having the polypeptide sequence of SEQ ID NO:103, and a light chain variable region having the polypeptide sequence of SEQ ID NO:104;
(46) a heavy chain variable region having the polypeptide sequence of SEQ ID NO:105, and a light chain variable region having the polypeptide sequence of SEQ ID NO:106;
(47) a heavy chain variable region having the polypeptide sequence of SEQ ID NO:107, and a light chain variable region having the polypeptide sequence of SEQ ID NO:108;
(48) a heavy chain variable region having the polypeptide sequence of SEQ ID NO:109, and a light chain variable region having the polypeptide sequence of SEQ ID NO:110;
(49) a heavy chain variable region having the polypeptide sequence of SEQ ID NO:111, and a light chain variable region having the polypeptide sequence of SEQ ID NO:112;
(50) a heavy chain variable region having the polypeptide sequence of SEQ ID NO:113, and a light chain variable region having the polypeptide sequence of SEQ ID NO:114;
(51) a heavy chain variable region having the polypeptide sequence of SEQ ID NO:115, and a light chain variable region having the polypeptide sequence of SEQ ID NO:116;
(52) a heavy chain variable region having the polypeptide sequence of SEQ ID NO:117, and a light chain variable region having the polypeptide sequence of SEQ ID NO:118;
(53) a heavy chain variable region having the polypeptide sequence of SEQ ID NO:119, and a light chain variable region having the polypeptide sequence of SEQ ID NO:120;
(54) a heavy chain variable region having the polypeptide sequence of SEQ ID NO:121, and a light chain variable region having the polypeptide sequence of SEQ ID NO:122;
(55) a heavy chain variable region having the polypeptide sequence of SEQ ID NO:123, and a light chain variable region having the polypeptide sequence of SEQ ID NO:124;
(56) a heavy chain variable region having the polypeptide sequence of SEQ ID NO:125, and a light chain variable region having the polypeptide sequence of SEQ ID NO:126;
(57) a heavy chain variable region having the polypeptide sequence of SEQ ID NO:127, and a light chain variable region having the polypeptide sequence of SEQ ID NO:128;
(58) a heavy chain variable region having the polypeptide sequence of SEQ ID NO:129, and a light chain variable region having the polypeptide sequence of SEQ ID NO:130;
(59) a heavy chain variable region having the polypeptide sequence of SEQ ID NO:131, and a light chain variable region having the polypeptide sequence of SEQ ID NO:132;
(60) a heavy chain variable region having the polypeptide sequence of SEQ ID NO:133, and a light chain variable region having the polypeptide sequence of SEQ ID NO:134;
(61) a heavy chain variable region having the polypeptide sequence of SEQ ID NO:135, and a light chain variable region having the polypeptide sequence of SEQ ID NO:136;
(62) a heavy chain variable region having the polypeptide sequence of SEQ ID NO:137, and a light chain variable region having the polypeptide sequence of SEQ ID NO:138;
(63) a heavy chain variable region having the polypeptide sequence of SEQ ID NO:139, and a light chain variable region having the polypeptide sequence of SEQ ID NO:140;
(64) a heavy chain variable region having the polypeptide sequence of SEQ ID NO:143, and a light chain variable region having the polypeptide sequence of SEQ ID NO:144;
(65) a heavy chain variable region having the polypeptide sequence of SEQ ID NO:145, and a light chain variable region having the polypeptide sequence of SEQ ID NO:146; or
(66) a heavy chain variable region having the polypeptide sequence of SEQ ID NO:147, and a light chain variable region having the polypeptide sequence of SEQ ID NO:148.

Embodiment 9 is the isolated monoclonal antibody or antigen-binding fragment thereof of any one of embodiments 1-8, wherein the monoclonal antibody or antigen-binding fragment thereof binds to human BTLA with a K_{D} of less than about 25 nM, 20 nM, 15 nM, 5 nM, 2 nM, 1 nM, or 0.5 nM.

Embodiment 10 is the isolated monoclonal antibody or antigen-binding fragment thereof of any one of embodiments 1-8, wherein the monoclonal antibody or antigen-binding fragment thereof is an agonist of human BTLA and activates downstream signaling from BTLA upon binding to BTLA.

Embodiment 11 is the isolated monoclonal antibody or antigen-binding fragment thereof of any one of embodiments 1-8, wherein the monoclonal antibody or antigen-binding fragment thereof is capable of inhibiting B cell proliferation and/or T cell and/or plasma cell activation.

Embodiment 12 is the isolated monoclonal antibody or antigen-binding fragment thereof of embodiment 11, wherein the T cell is selected from the group consisting of a CD4 T-cell, a CD8 T-cell, a Th1 T-cell, a TFH T-cell, an αβ T-cell, and a γδ T-cell.

Embodiment 13 is the isolated monoclonal antibody or antigen-binding fragment of any one of embodiments 1-8, wherein the monoclonal antibody or antigen-binding fragment thereof is capable of blocking or abolishing BTLA binding to herpes virus entry mediator (HVEM).

Embodiment 14 is the isolated monoclonal antibody or antigen-binding fragment thereof of any one of embodiments 1-8, wherein the monoclonal antibody or antigen-binding fragment thereof is capable of increasing pro-inflammatory cytokine production; and/or mediating the recruitment of conjugated drugs; and/or forming a bispecific antibody with another mAb or antigen-binding fragment thereof with cancer-killing effect.

Embodiment 15 is the isolated monoclonal antibody or antigen-binding fragment thereof of embodiment 14, wherein the pro-inflammatory cytokine is selected from interferon γ (IFNγ), interleukin-2 (IL-2), C—X—C motif chemokine ligand 9 (CXCL9), C—X—C motif chemokine ligand 10 (CXCL10), interleukin 1β (IL1β), and/or tumor necrosis factor a (TNFα).

Embodiment 16 is the isolated monoclonal antibody or antigen-binding fragment thereof of any one of embodiments 1-15, wherein the monoclonal antibody or antigen-binding fragment thereof specifically binds cynomolgus BTLA.

Embodiment 17 is an isolated bispecific antibody or antigen-binding fragment thereof comprising the monoclonal antibody or antigen-binding fragment thereof of any one of embodiments 1-16.

Embodiment 18 is an isolated nucleic acid encoding the monoclonal antibody or antigen-binding fragment of any one of embodiments 1-16.

Embodiment 19 is an isolated nucleic acid encoding the bispecific antibody or antigen-binding fragment thereof of embodiment 17.

Embodiment 20 is a vector comprising the isolated nucleic acid of embodiment 18 or 19.

Embodiment 21 is a host cell comprising the vector of embodiment 20.

Embodiment 22 is a pharmaceutical composition, comprising the isolated monoclonal antibody or antigen-binding fragment of any one of embodiments 1-16 or the bispecific antibody or antigen-binding fragment thereof of embodiment 17 and a pharmaceutically acceptable carrier.

Embodiment 23 is a method of targeting BTLA on a cancer cell surface, and/or treating a cancer in a subject in need thereof, comprising administering to the subject in need thereof the pharmaceutical composition of embodiment 22.

Embodiment 24 is the method of embodiment 23, wherein the cancer is a solid tumor, preferably a solid tumor with infiltrating T cells, more preferably a solid tumor with infiltrating T effector cells, more preferably a solid tumor with T effector cells expressing BTLA, most preferably a solid tumor with infiltrating T effector cells expressing BTLA and BTLA ligand HVEM highly expressing in the tumor microenvironment.

Embodiment 25 is the method of embodiment 23 or 24, wherein the cancer is selected from the group consisting of melanoma, lung cancer, renal cell carcinoma, and liver cancer.

Embodiment 26 is the method of any one of claims 23-25, wherein the subject comprises BTLA-expressing T cells, B cells, dendritic cells (DCs), or natural killer (NK) cells.

Embodiment 27 is the method of any one of embodiments 23-26, wherein the pharmaceutical composition further comprises a second anti-cancer agent.

Embodiment 28 is the method of embodiment 27, wherein the second anti-cancer agent is an anti-PD-1 antibody or antigen-binding fragment thereof.

Embodiment 29 is a method of producing the monoclonal antibody or antigen-binding fragment of any one of embodiments 1-16 or the bispecific antibody or antigen-binding fragment thereof of embodiment 17, comprising culturing a cell comprising a nucleic acid encoding the monoclonal antibody or antigen-binding fragment thereof or bispecific antibody or antigen-binding fragment thereof under conditions to produce the monoclonal antibody or antigen-binding fragment thereof or bispecific antibody or antigen-binding fragment thereof and recovering the monoclonal antibody or antigen-binding fragment thereof or bispecific antibody or antigen-binding fragment thereof from the cell or culture.

Embodiment 30 is a method of producing a pharmaceutical composition comprising the monoclonal antibody or antigen-binding fragment thereof of any one of embodiments 1-16 or the bispecific antibody or antigen-binding fragment thereof of embodiment 17, comprising combining the monoclonal antibody or antigen-binding fragment thereof or bispecific antibody or antigen-binding fragment thereof with a pharmaceutically acceptable carrier to obtain the pharmaceutical composition.

Embodiment 31 is a method of determining the level of BTLA in a subject, the method comprising:
a. obtaining a sample from the subject:
b. contacting the sample with the isolated monoclonal antibody or antigen-binding fragment thereof of any one of embodiments 1-16; and
c. determining the level of the BTLA in the subject.

Embodiment 32 is the method of embodiment 31, wherein the sample is a tissue sample or a blood sample, optionally wherein the tissue sample is a cancer tissue sample.

Embodiment 33 is the method of embodiment 31, wherein the sample comprises T cells, B cells, dendritic cells (DCs), or natural killer (NK) cells.

### EXAMPLES

### Example 1: Identification of Anti-Human BTLA Blocking Antibodies that Cross-React With Cynomolgus Monkey BTLA

This example describes the identification of two mouse lead antibody candidates—HFB6-2 and HFB6-3 (sequences described in Tables 2-5 below)—based on their ability to reverse HVEM-mediated T cell suppression to a similar extent as a benchmark anti-BTLA blocking antibody (referred to herein as JS004).

TABLE 2
CDR regions 1-3 of heavy chain for anti-BTLA
mAbs as determined by IMGT method.

| | | SEQ | | SEQ | | SEQ |
|---|---|---|---|---|---|---|
| | | ID | | ID | | ID |
| VH | HC CDR1 | NO: | HC CDR2 | NO: | HC CDR3 | NO: |
| HFB6-2 | CTASGVNI | 1 | GRIDPAN | 2 | CAVNDGY | 3 |
| | KDTYMYW | | GYTRYD | | FEYFDVW | |
| HFB6-3 | CTDSGFN | 7 | GRIDPAN | 8 | CAREDGY | 9 |
| | IKDTYIHW | | GFTRYD | | PYYTLDCW | |

TABLE 3
CDR regions 1-3 of light chain for anti-
BTLA mAbs as determined by
IMGT method.

| | | SEQ | | SEQ | | SEQ |
|---|---|---|---|---|---|---|
| | | ID | VL | ID | VL | ID |
| VL | VL CDR1 | NO: | CDR2 | NO: | CDR3 | NO: |
| HFB6-2 | CKSSQSLLD | 4 | LVSK | 5 | CWQDTH | 6 |
| | SDGKPYLNW | | LDS | | FPRTF | |
| HFB6-3 | CKSSQSLLD | 10 | LVSK | 11 | CWQGTH | 12 |
| | VDGKTYLNW | | LDS | | FPRTF | |

TABLE 4
Sequences of heavy chain variable regions
for anti-BTLA mAbs.

| | | SEQ |
|---|---|---|
| mAb | | ID |
| clones | VH sequence | NO: |
| HFB6-2 | EVQLQQSGAELVKPGASVKLSCTASGVNIK | 13 |
| | DTYMYWVKQRPEQGLEWIGRIDPANGYTRY | |
| | DPKFQDKATVTADTSSNTAHLQLSSLTSED | |
| | TAVYYCAVNDGYFEYFDVWGAGTTVTVSS | |
| HFB6-3 | EVQLQQSGAELVKPGASVKLSCTDSGFNIK | 15 |
| | DTYIHWVTQRPQQGLEWIGRIDPANGFTRY | |
| | DPKFQGKATITSDTSSNTAYLQLSSLTSED | |
| | TAVYYCAREDGYPYYTLDCWGQGTSVTVSS | |

TABLE 5
Sequences of light chain variable regions
for anti-BTLA mAbs.

| | | SEQ |
|---|---|---|
| mAb | | ID |
| clones | VL sequence | NO: |
| HFB6-2 | DVVMTQAPLTLSVTIGQPASISCKSSQSLLDSDG | 14 |
| | KPYLNWLLQRPGQSPKRLIYLVSKLDSGVPDRFT | |
| | GSGSGTDFTLKISRVEAEDLGVYYCWQDTHFPRT | |
| | FGGGTKLEIK | |
| HFB6-3 | DVVMTQSPLTLSLTIGQPASISCKSSQSLLDVDG | 16 |
| | KTYLNWFLQRPGQSPKRLIYLVSKLDSGVPDRFT | |
| | GSGSGTDFTLKISRVEAEDLGVYYCWQGTHFPRT | |
| | FGGGTKLEIK | |

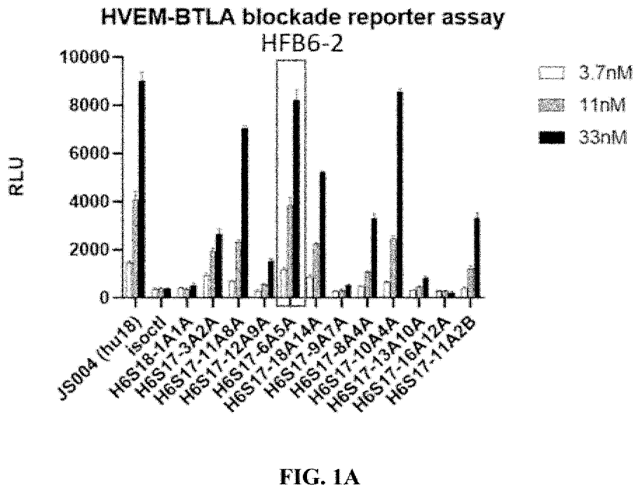

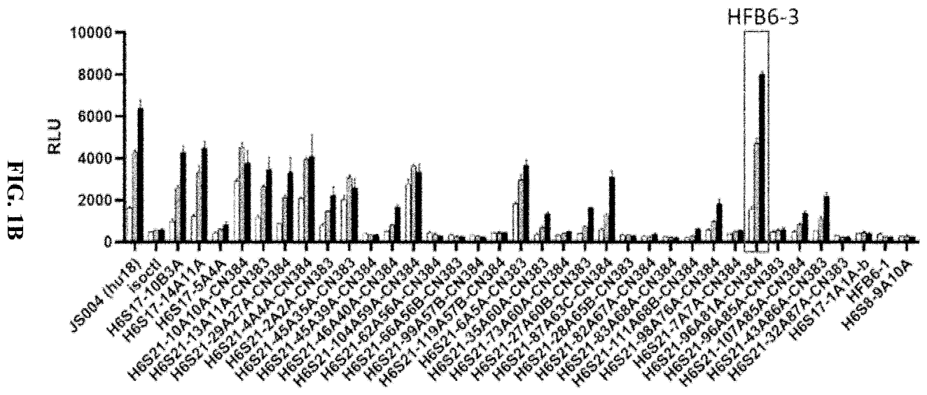

In the HVEM-BTLA blockade reporter assay, a CHO cell line engineered to express a TCR activator (TCRa) and HVEM (the ligand of BTLA) was co-cultured with a Jurkat reporter cell line that expressed BTLA and a luciferase reporter driven by an NFAT binding promoter. A panel of anti-BTLA antibody candidates was screened based on their ability to relieve HVEM-BTLA interaction and restore luciferase reporter signal (FIGS. 1A-1B). HFB6-2 and HFB6-3 were able to restore TCR activation in this reporter assay to a similar extent as benchmark JS004.

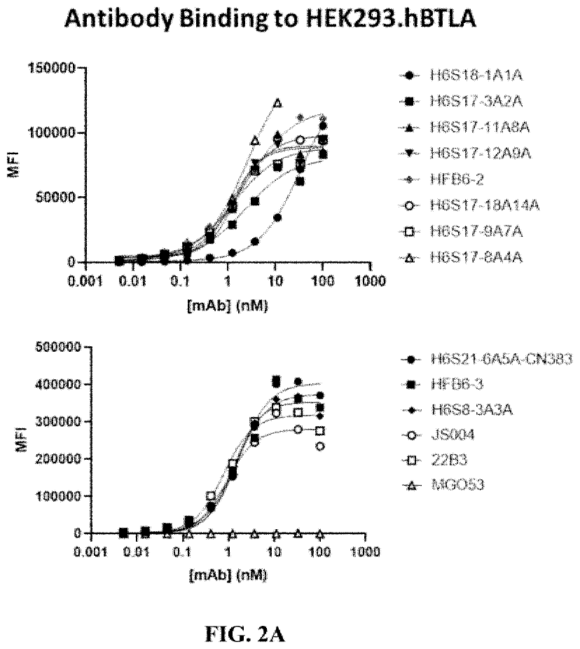

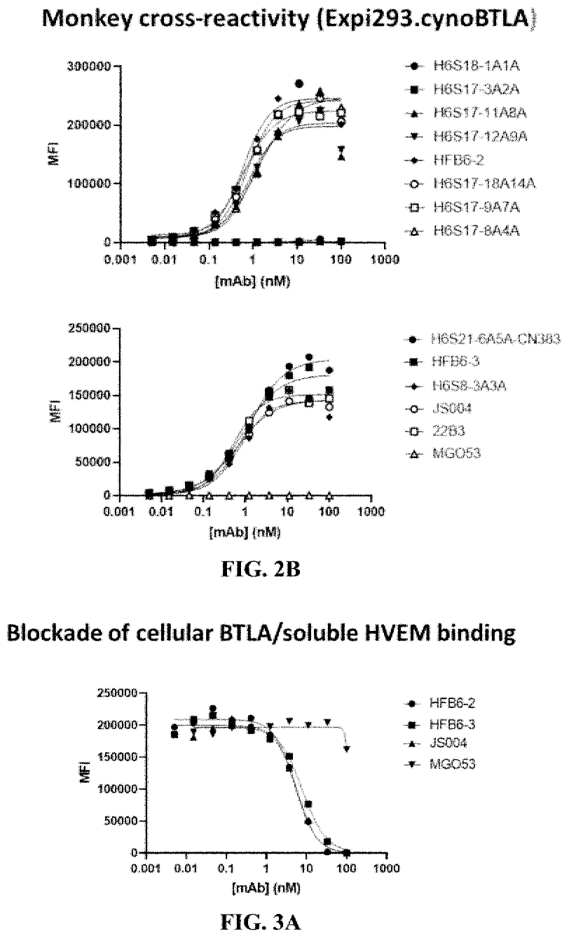

In FIGS. 2A-2B, HEK293 cells overexpressing human BTLA and Expi293 cells overexpressing cynomolgus BTLA were incubated with different concentrations of anti-BTLA parental antibodies. Anti-BTLA antibody binding to target cells was quantified with an AF647-conjugated anti-human IgG Fc 2nd antibody by FACS. HFB6-2 and HFB6-3 were shown to bind to human BTLA and cyno BTLA with single digit nM and sub-nM EC₅₀ (Table 6).

TABLE 6
HFB-2 and HFB-6 binding to human and cyno BTLA

| Antibody | hBTLA binding EC₅₀ (nM) | CynoBTLA binding EC₅₀ (nM) |
|---|---|---|
| HFB6-2 | 2.342 | 0.6135 |
| HFB6-3 | 1.446 | 0.8231 |
| JS004 | 0.8045 | 0.5611 |

### Example 2: HFB6-2 and HFB6-3 are Anti-BTLA Blockers

This example demonstrates that HFB6-2 and HFB6-3 blocked the interaction between recombinant HVEM soluble protein with cellular BTLA and relieved HVEM-mediated TCR inhibition in T cells.

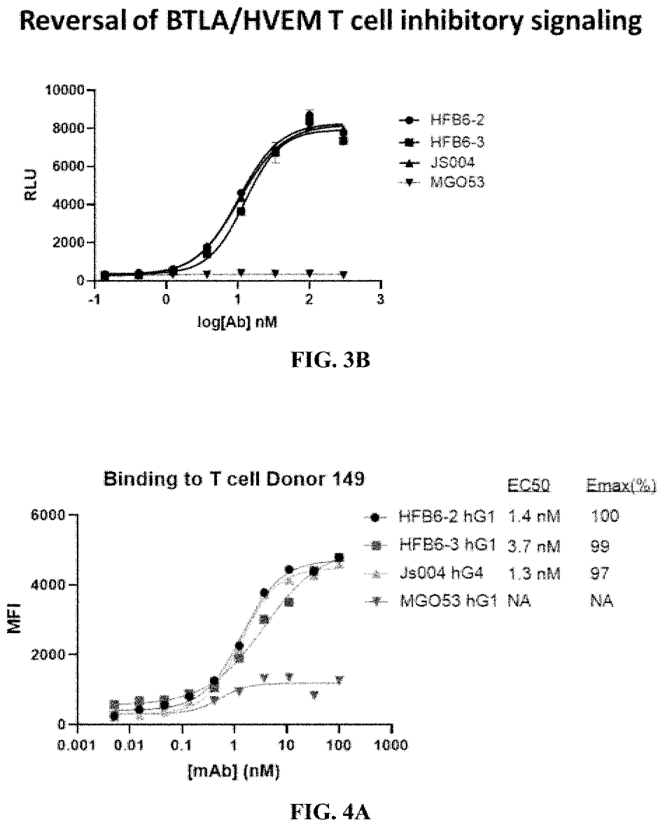

In FIG. 3A, recombinant HVEM soluble protein was conjugated with DyLight 650 (DL650) fluorophore. HEK293 cells overexpressing hBTLA (HEK293.BTLA) were preincubated with increasing concentrations of HFB6-2 and HFB6-3 at 4° C. for 30 minutes and then with DL650-conjugated HVEM for an additional 30 minutes. After washing, soluble HVEM remaining associated with BTLA on HEK293.BTLA cells was measured by FACS. HFB6-2 and HFB6-3 blocked soluble HVEM and cellular BTLA interaction with single digit nM IC₅₀.

In FIG. 3B, the functional blocker activity of HFB6-2 and HFB6-3 was evaluated in a HVEM-BTLA blockade reporter assay. In this assay, a CHO cell line engineered to express a TCR activator (TCRa) and HVEM (the ligand of BTLA) was co-cultured with a Jurkat reporter cell line that expressed BTLA and a luciferase reporter driven by an NFAT binding promoter. Application of HFB6-2 and HFB6-3 restored luciferase reporter signal in a dose-dependent manner similar as benchmark JS004.

TABLE 7
HFB6-2 and HFB6-3 blocking ability

| | | BTLA-HVEM |
|---|---|---|
| | Soluble HVEM-hBTLA blocking, | blockade reporter |
| Antibody | IC₅₀ (nM) | assay, EC50 (nM) |
| HFB6-2 | 5.451 | 10.19 |
| HFB6-3 | 7.706 | 12.47 |
| JS004 | 4.323 | 10.37 |

### Example 3: HFB6-2 and HFB6-3 Bind to Primary T and B Cells

This example demonstrates HFB6-2 and HFB6-3 bound to primary T cells and B cells with single-digit nM EC₅₀ and sub-nM EC₅₀ respectively.

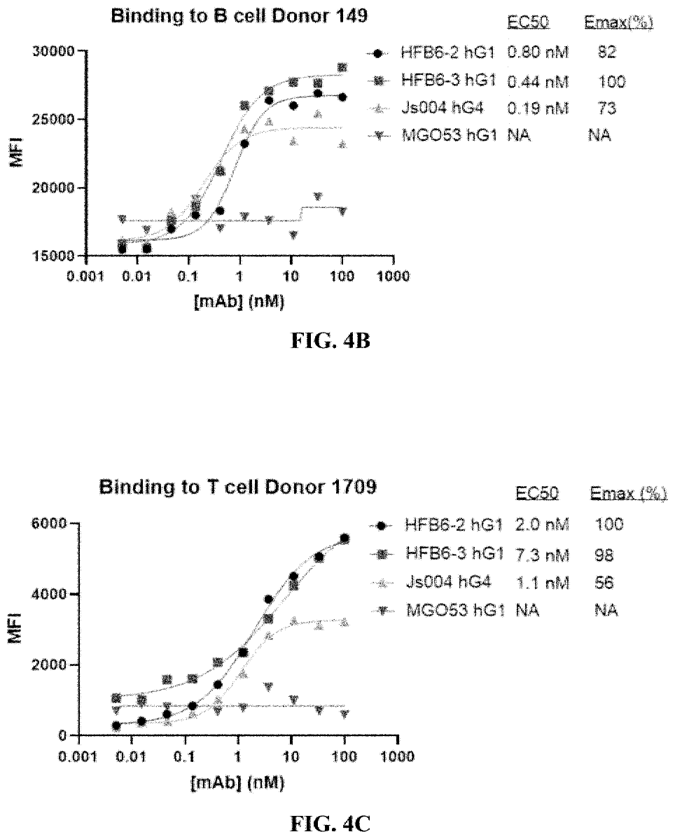

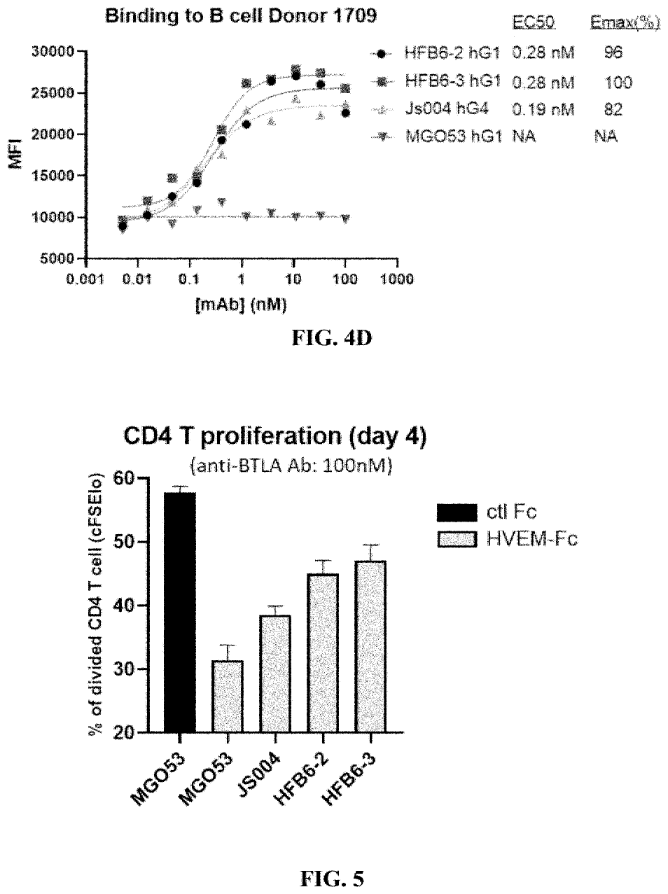

In FIGS. 4A-4D, primary T cells and B cells were isolated from human PBMC with EasySep™ Human T Cell Isolation Kit and B cell isolation kit (STEMCELL Technologies: Vancouver, Canada). Isolated T cells or B cells were incubated with different concentrations of anti-BTLA parental antibodies. Anti-BTLA antibody binding to target cells was quantified with an AF647-conjugated anti-human IgG Fc 2nd antibody by FACS.

### Example 4: HFB6-2 and HFB6-3 Reverse HVEM-Mediated Suppression on Primary CD4+ T-Cell Proliferation

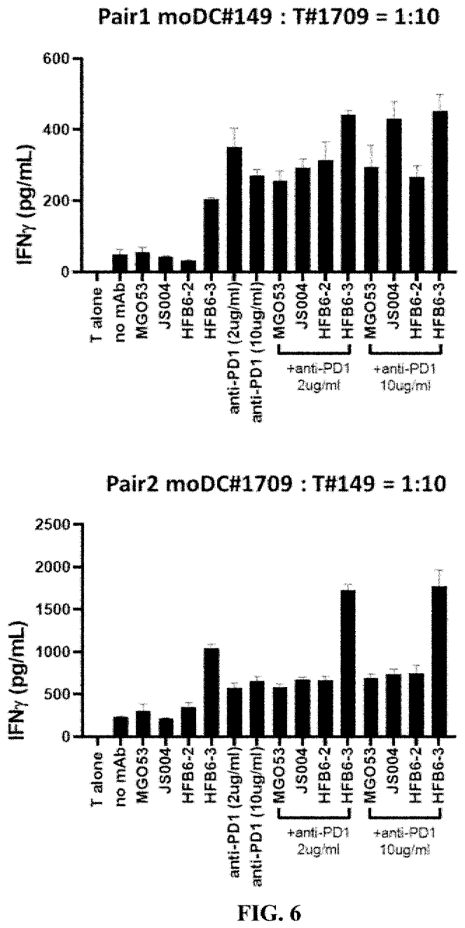

In FIGS. 5A-5B, a cell culture plate was coated with anti-CD3 antibody together with recombinant HVEM-Fc fusion protein or control Fc protein (ctl-Fc) at 4° C. overnight. Primary CD4⁺ T cells were isolated from human PBMC with EasySep™ Human CD4+ T Cell Isolation Kit (STEMCELL Technologies) and labeled with CFSE. CD4+ T cells were added on to anti-CD3 and HVEM-Fc coated plate and incubated with anti-CD28 and anti-BTLA blocking antibodies and isotype-matched control antibody (MGO53). After 4 days. CD4⁺ T cell proliferation was quantified by CFSE dilution (% CFSElo) related to non-stimulated CD4⁺ T cells. HFB6-2 and HFB6-3 reversed HVEM-mediated suppression on primary CD4+T cell proliferation.

### Example 5: HFB6-3 Increased IFNγ Production and Synergized with Anti-PD1 in Mixed Lymphocyte Reaction (MLR) Assay

In the FIG. 6 MLR assay. CD14⁺ monocytes were isolated from one PBMC donor using EasySep™ Human Monocyte Isolation Kit, differentiated into immature dendritic cells with GM-CSF and IL-4 for 5 days and then into mature dendritic cells (moDC) with LPS for additional 2 days. Afterwards, moDCs were mixed with primary T cells from a second donor at 1:10 ratio in the presence of anti-BTLA antibody and/or anti-PD1 antibody. After 5 days. IFNγ in the supernatant was measured by AlphaLISA. In both moDC:T cell pairs tested. HFB6-3 alone enhanced IFNγ production compared with isotype-control (MGO53). HFB6-3 in combination with anti-PD1 further enhanced IFNγ production.

### Example 6: HFB6-3 Alone or in Combination with Anti-PD1 Induced IFNγ and Pro-Inflammatory Cytokine Production in Primary Dissociated Tumor Culture

Primary tumor tissue from lung and melanoma cancer patients was dissociated into single cell suspension with human Tumor Dissociation Kit (Miltenyi Biotec; Bergisch Gladbach. Germany). BTLA and PD1 expression on the tumor infiltrating lymphocytes (TILs) was measured by FACS. 1×10⁵ dissociated tumor cells were cultured with HFB6-3 and/or anti-PD1 (10 μg/ml) in the presence of anti-CD3 (10 ng/ml). Supernatants were harvested at day 3 post treatment and analyzed with Human Cytokine/Chemokine 48-Plex Discovery Assay® (HD48) (Eve Technologies: Calgary. Canada). Supernatants were also harvested on day 7 post treatment and analyzed with IFNγ ELISA (R&D systems: Minneapolis, MN). HFB6-3 showed single agent effect as well as synergistic effect with anti-PD1 in upregulating multiple pro-inflammatory cytokines in multiple primary lung and melanoma tumor cultures.

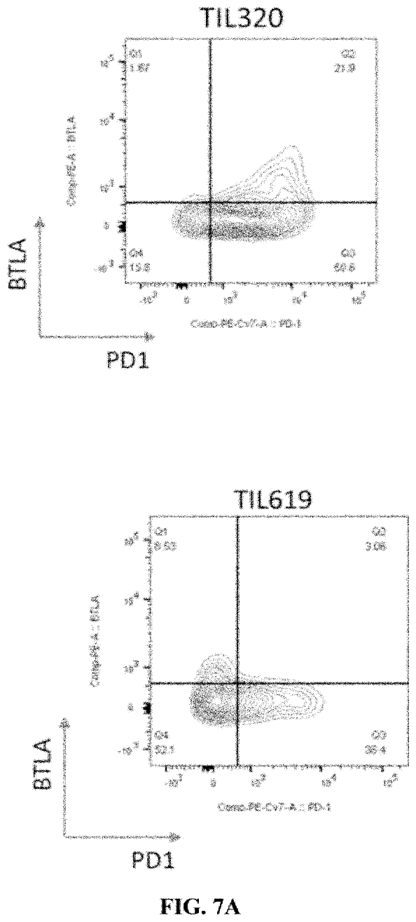

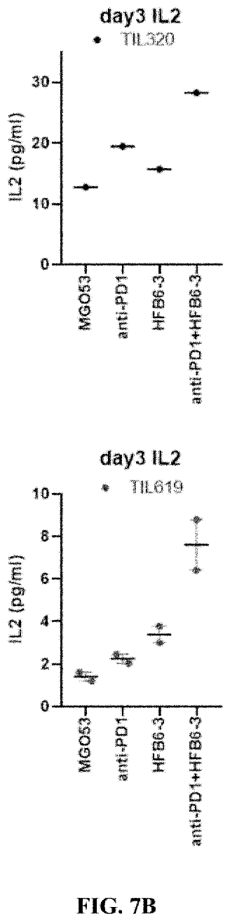

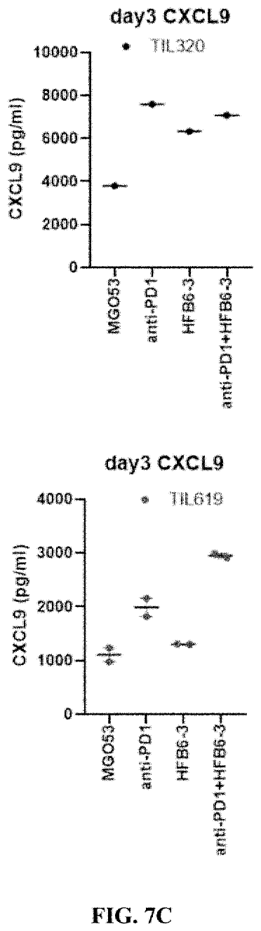

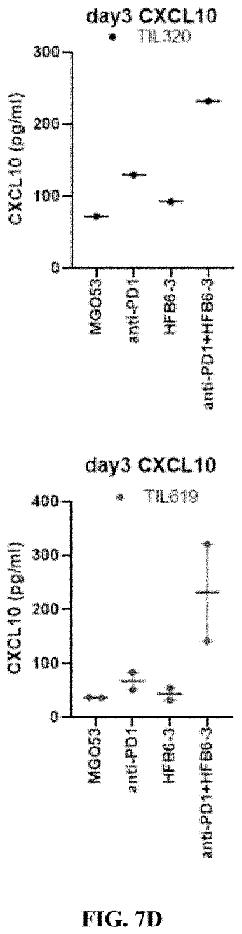

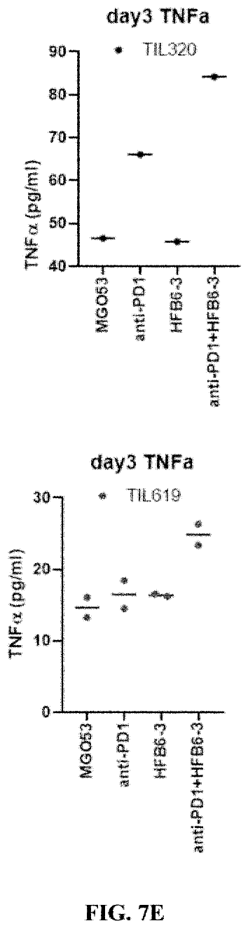

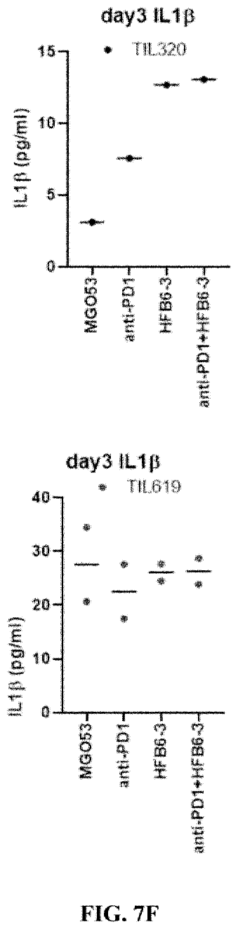

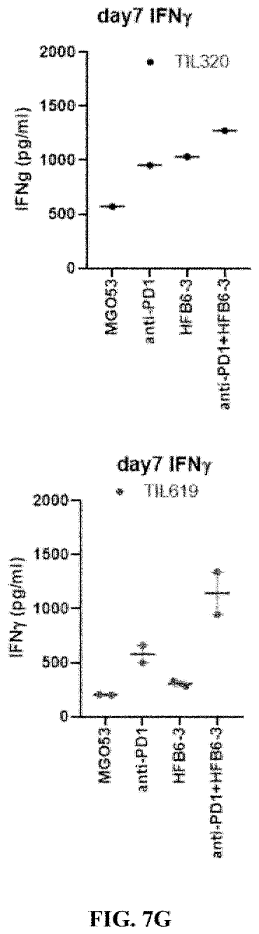

In FIGS. 7A-7G, dissociated tumor tissue from lung patient TIL 320 has higher % of BTLA+ CD4+T cells than TIL619. In TIL320, HFB6-3 treatment alone increased IL-2, CXCL9, IL1β and IFNγ production. Combination of HFB6-3 and anti-PD1 further enhanced IL-2, CXCL10, TNFα and IFNγ production compared with either single agent treatment (FIGS. 7B, 7D, 7E, and 7G). In TIL619, HFB6-3 did not show single agent effect. However. HFB6-3 in combination with anti-PD1 further enhanced IL-2, CXCL9, CXCL10, TNFα and IFNγ production compared with anti-PD1 treatment alone (FIGS. 7B, 7C, 7D, 7E, and 7G).

### Example 7: Desirable PK Profiles of HFB6-2 and HFB6-3 in Wild-Type C57BL/6 Mice and hBTLA Knock-In Mice

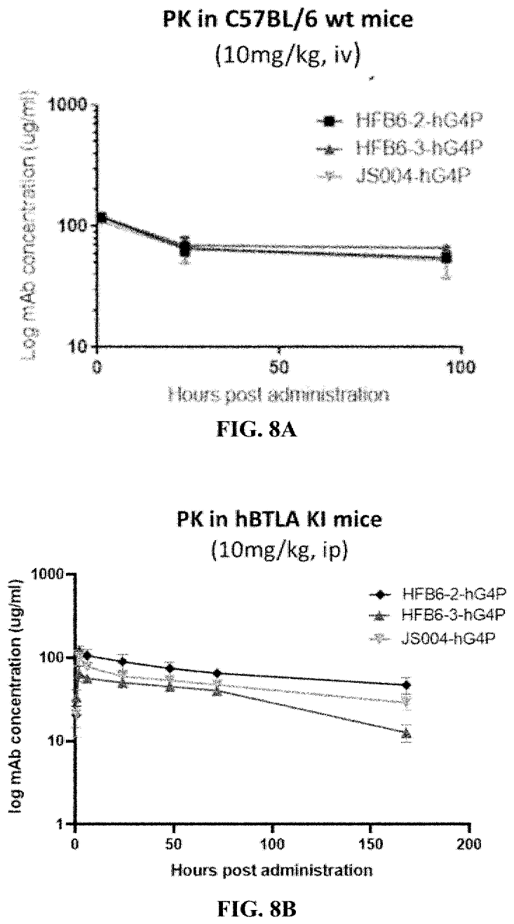

In this experiment (FIGS. 8A and 8B), a single dose (10 mg/kg) of HFB6-2, HFB6-3 and benchmark JS004 was injected into wild type C57BL/6 mice (i.v. route, FIG. 8A) and hBTLA knock-in mice (i.p. route, hBTLA KI, FIG. 8B). At 1 hour, 24 hours, and 72 hours, blood was drawn for the wild type C57BL/6 mice. At 0.5 hours, 6 hours, 24 hours, 48 hours, 72 hours, 96 hours, and 168 hours, blood was drawn from hBTLA KI mice. Plasma concentration of selected antibodies were measured by ELISA.

Single i.v. or i.p, administration of HFB6-2 or HFB6-3 (10 mg/kg) to wt C57BL/6 or hBTLA KI mice resulted in plasma concentrations >40 μg/ml for at least 72 hours (FIGS. 8A and 8B); supportive of q3d or less frequent administration for efficacy evaluation.

### Example 8: Functional Profiling of HFB6-2 and HFB6-3 Humanized Antibodies

21 humanized variants of HFB6-2 (e.g., HFB6-2hz1 to -hz21) were generated based on standard humanization protocols, and the resulting humanized variants were tested to identify high affinity binders which maintain parental HFB6-2 blocking activity.

In FIG. 9, binding of humanized HFB6-2 variants to recombinant human BTLA was evaluated in an ELISA binding assay. All the tested HFB6-2 humanized variants maintain parental HFB6-2 antibody binding affinity in ELISA assay. Among them, HFB6-2hz16, HFB6-2hz18 and HFB6-2hz20 showed the closest functional blocking activity to parental HFB6-2 in the HVEM-BTLA blockade reporter assay (FIG. 9 and Table 8).

TABLE 8
EC₅₀ for humanized HFB6-2 variants
in ELISA and Reporter Assays

| Antibody | ELISA EC₅₀ (nM) | Reporter EC₅₀ (nM) |
|---|---|---|
| JS004-hG4P | 2.428 | 13.12 |
| HFB6-2-hG4P | 2.345 | 11.47 |
| HFB6-2hz8-hG4P | 3.96 | 66.85 |
| HFB6-2hz9-hG4P | 3.292 | 36.67 |
| HFB6-2hz0-hG4P | 3.214 | 46.99 |
| HFB6-2hz11-hG4P | 3.664 | 19.41 |
| HFB6-2hz12-hG4P | 5.053 | 43.21 |
| HFB6-2hz15-hG4P | 2.744 | 19.57 |
| HFB6-2hz16-hG4P | 2.392 | 10.84 |
| HFB6-2hz17-hG4P | 2.404 | 18.22 |
| HFB6-2hz18-hG4P | 2.242 | 10.82 |
| HFB6-2hz19-hG4P | 2.549 | 22.11 |
| HFB6-2hz20-hG4P | 2.507 | 9.774 |

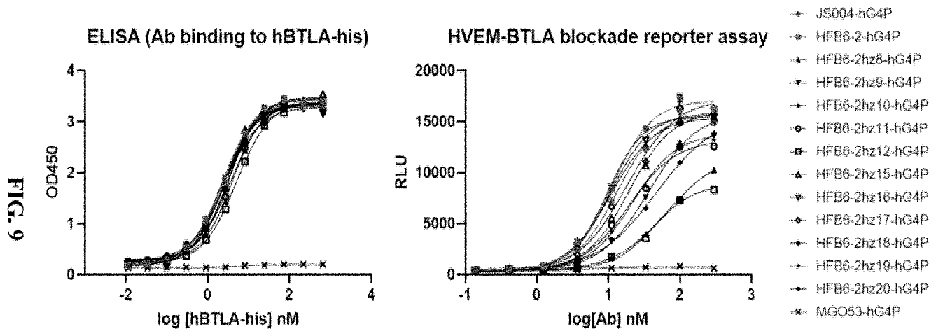

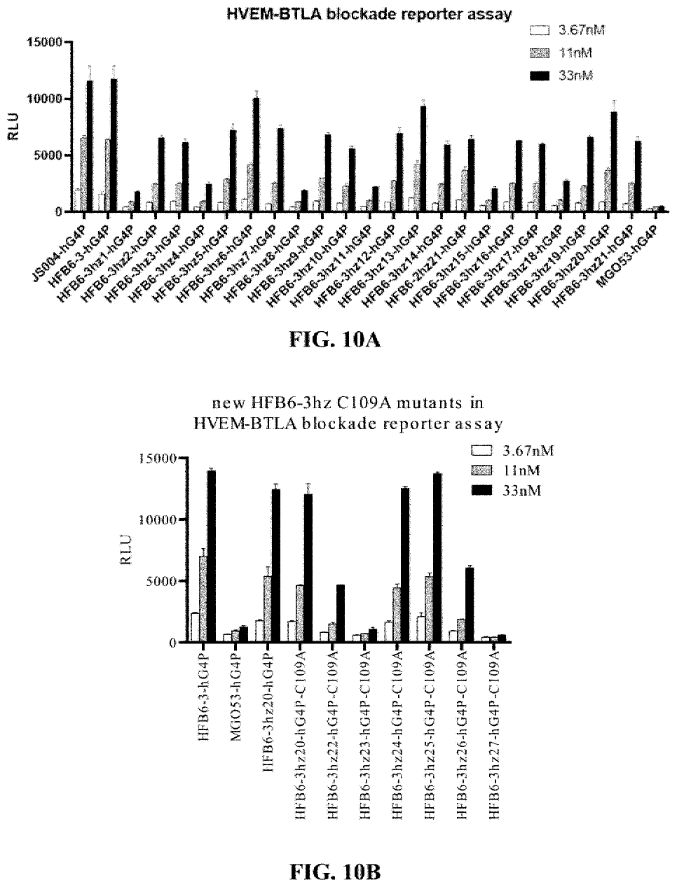

45 humanized variants of HFB6-3 including Cys mutants on HCDR3 (e.g., HFB6-3hz1-hG4P to HFB6-3hz21-hG4P, HFB6-3hz20-hG4P-C109A/S/T/V) were generated based on standard humanization protocols. Since there is an unpaired Cys on HCDR3 of HFB6-3, C109 on several humanized variants were mutated to A/S/T/V. In the HVEM-BTLA blockade reporter assay (FIGS. 10A-10C), multiple humanized HFB6-3 variants, including but not limited to HFB6-3hz6-hG4P, HFB6-3hz13-hG4P, HFB6-3hz20-hG4P, HFB6-3hz20-hG4P-C109A, HFB6-3hz24-hG4P-C109A, HFB6-3hz25-hG4P-C109A, HFB6-3hz24-hG4P-C109S, HFB6-3hz25-hG4P-C109S, restored TCR activation close to the level of parental HFB6-3.

The humanized antibody sequences are provided below in Tables 9-12:

TABLE 9
Sequences of humanized heavy chain variable regions for HFB6-2 and
HFB6-3 mAbs.

| | | SEQ |
|---|---|---|
| | | ID |
| VH | VH sequence | NO: |
| HFB6-2hz1-hG4P | QVQLVQSGAEVKKPGASVKVSCKASGVNIKDTYMYWVRQAPGQGLE | 17 |
| | WIGRIDPANGYTRYAQKFQGRVTMTADTSTSTVYMELSSLRSEDTAV | |
| | YYCAVNDGYFEYFDVWGQGTTVTVSS | |
| HFB6-2hz2-hG4P | QVQLVQSGAEVKKPGASVKVSCKASGVNIKDTYMYWVRQAPGQGLE | 19 |
| | WIGRIDPANGYTRYAQKFQGRATVTADTSTSTVYLELSSLRSEDTAVY | |
| | YCAVNDGYFEYFDVWGQGTTVTVSS | |
| HFB6-2hz3-hG4P | QVQLVQSGAEVKKPGSSVKVSCKASGVNIKDTYMYWVRQAPGQGLE | 21 |
| | WIGRIDPANGYARYAQKFQGRATITADESTSTAYLELSSLRSEDTAVY | |
| | YCAVNDGYFEYFDVWGQGTTVTVSS | |
| HFB6-2hz4-hG4P | QVQLVQSGAEVKKPGSSVKVSCKASGVNIKDTYMYWVRQAPGQGLE | 23 |
| | WIGRIDPANGYARYAQKFQGRATVTADKSTSTAYLELSSLRSEDTAVY | |
| | YCAVNDGYFEYFDVWGQGTTVTVSS | |
| HFB6-2hz5-hG4P | EVQLLESGGGLVQPGGSLRLSCAASGVNIKDTYMYWVRQAPGKGLE | 25 |
| | WIGRIDPANGYTRYADSVKGRFTISADNSKNTLYLQMNSLRAEDTAV | |
| | YYCAVNDGYFEYFDVWGQGTTVTVSS | |
| HFB6-2hz6-hG4P | QVQLQESGPGLVKPSGTLSLTCAASGVNIKDTYMYWVRQPPGKGLEW | 27 |
| | IGRIDPANGYTRYNPSFKSRATVSADKSKNQASLKLSSLTAADTAVY | |
| | YCAVNDGYFEYFDVWGQGTTVTVSS | |
| HFB6-2hz7-hG4P | EVQLVQSGAEVKKPGESLKISCKGSGVNIKDTYMYWVRQMPGKGLEW | 29 |
| | IGRIDPANGYTRYSPSFQGQATVSADKSISTAYLQLSSLKASDTAMY | |
| | YCAVNDGYFEYFDVWGQGTTVTVSS | |
| HFB6-2hz8-hG4P | QVQLVQSGAEVKKPGASVKVSCKASGVNIKDTYMYWVRQAPGQGLE | 31 |
| | WIGRIDPANGYTRYAQKFQGRVTMTADTSTSTVYMELSSLRSEDTA | |
| | VYYCAVNDGYFEYFDVWGQGTTVTVSS | |
| HFB6-2hz9-hG4P | QVQLVQSGAEVKKPGASVKVSCKASGVNIKDTYMYWVRQAPGQGLEW | 33 |
| | IGRIDPANGYTRYAQKFQGRATVTADTSTSTVYLELSSLRSEDTAVY | |
| | YCAVNDGYFEYFDVWGQGTTVTVSS | |
| HFB6-2hz10-hG4P | QVQLVQSGAEVKKPGSSVKVSCKASGVNIKDTYMYWVRQAPGQGLEW | 35 |
| | IGRIDPANGYARYAQKFQGRATITADESTSTAYLELSSLRSEDTAVY | |
| | YCAVNDGYFEYFDVWGQGTTVTVSS | |
| HFB6-2hz11-hG4P | QVQLVQSGAEVKKPGSSVKVSCKASGVNIKDTYMYWVRQAPGQGLEW | 37 |
| | IGRIDPANGYARYAQKFQGRATVTADKSTSTAYLELSSLRSEDTAVY | |
| | YCAVNDGYFEYFDVWGQGTTVTVSS | |
| HFB6-2hz12-hG4P | EVQLLESGGGLVQPGGSLRLSCAASGVNIKDTYMYWVRQAPGKGLE | 39 |
| | WIGRIDPANGYTRYADSVKGRFTISADNSKNTLYLQMNSLRAEDTAV | |
| | YYCAVNDGYFEYFDVWGQGTTVTVSS | |
| HFB6-2hz13-hG4P | QVQLQESGPGLVKPSGTLSLTCAASGVNIKDTYMYWVRQPPGKGLEW | 41 |
| | IGRIDPANGYTRYNPSFKSRATVSADKSKNQASLKLSSLTAADTAVY | |
| | YCAVNDGYFEYFDVWGQGTTVTVSS | |
| HFB6-2hz14-hG4P | EVQLVQSGAEVKKPGESLKISCKGSGVNIKDTYMYWVRQMPGKGLEW | 43 |
| | IGRIDPANGYTRYSPSFQGQATVSADKSISTAYLQLSSLKASDTAMY | |
| | YCAVNDGYFEYFDVWGQGTTVTVSS | |
| HFB6-2hz15-hG4P | QVQLVQSGAEVKKPGASVKVSCKASGVNIKDTYMYWVRQAPGQGLE | 45 |
| | WIGRIDPANGYTRYAQKFQGRVTMTADTSTSTVYMELSSLRSEDTAV | |
| | YYCAVNDGYFEYFDVWGQGTTVTVSS | |
| HFB6-2hz16-hG4P | QVQLVQSGAEVKKPGASVKVSCKASGVNIKDTYMYWVRQAPGQGLEW | 47 |
| | IGRIDPANGYTRYAQKFQGRATVTADTSTSTVYLELSSLRSEDTAVY | |
| | YCAVNDGYFEYFDVWGQGTTVTVSS | |
| HFB6-2hz17-hG4P | QVQLVQSGAEVKKPGSSVKVSCKASGVNIKDTYMYWVRQAPGQGLEW | 49 |
| | IGRIDPANGYARYAQKFQGRATITADESTSTAYLELSSLRSEDTAVY | |
| | YCAVNDGYFEYFDVWGQGTTVTVSS | |
| HFB6-2hz18-hG4P | QVQLVQSGAEVKKPGSSVKVSCKASGVNIKDTYMYWVRQAPGQGLEW | 51 |
| | IGRIDPANGYARYAQKFQGRATVTADKSTSTAYLELSSLRSEDTAVY | |
| | YCAVNDGYFEYFDVWGQGTTVTVSS | |
| HFB6-2hz19-hG4P | EVQLLESGGGLVQPGGSLRLSCAASGVNIKDTYMYWVRQAPGKGLE | 53 |
| | WIGRIDPANGYTRYADSVKGRFTISADNSKNTLYLQMNSLRAEDTAV | |
| | YYCAVNDGYFEYFDVWGQGTTVTVSS | |
| HFB6-2hz20-hG4P | QVQLQESGPGLVKPSGTLSLTCAASGVNIKDTYMYWVRQPPGKGLEW | 55 |
| | IGRIDPANGYTRYNPSFKSRATVSADKSKNQASLKLSSLTAADTAVY | |
| | YCAVNDGYFEYFDVWGQGTTVTVSS | |
| HFB6-2hz21-hG4P | EVQLVQSGAEVKKPGESLKISCKGSGVNIKDTYMYWVRQMPGKGLEW | 57 |
| | IGRIDPANGYTRYSPSFQGQATVSADKSISTAYLQLSSLKASDTAMY | |
| | YCAVNDGYFEYFDVWGQGTTVTVSS | |
| HFB6-3hz1-hG4P | QVQLVQSGAEVKKPGASVKVSCKDSGFNIKDTYIHWVRQAPGQGLEW | 59 |
| | IGRIDPANGFTRYAQKFQGRVTMTSDTSTSTVYMELSSLRSEDTAVY | |
| | YCAREDGYPYYTLDCWGQGTTVTVSS | |
| HFB6-3hz2-hG4P | QVQLVQSGAEVKKPGASVKVSCKDSGFNIKDTYIHWVRQAPGQGLEW | 61 |
| | IGRIDPANGFTRYAQKFQGRATITSDTSTSTVYLELSSLRSEDTAVY | |
| | YCAREDGYPYYTLDCWGQGTTVTVSS | |
| HFB6-3hz3-hG4P | QVQLVQSGAEVKKPGSSVKVSCKDSGFNIKDTYIHWVRQAPGQGLEWI | 63 |
| | GRIDPANGFARYAQKFQGRATITSDESTSTAYLELSSLRSEDTAVYYC | |
| | AREDGYPYYTLDCWGQGTTVTVSS | |
| HFB6-3hz4-hG4P | QVQLVQSGAEVKKPGSSVKVSCKDSGFNIKDTYIHWVRQAPGQGLEWI | 65 |
| | GRIDPANGFARYAQKFQGRATITSDKSTSTAYLELSSLRSEDTAVYYC | |
| | AREDGYPYYTLDCWGQGTTVTVSS | |
| HFB6-3hz5-hG4P | EVQLVESGGGLVQPGGSLRLSCADSGFNIKDTYIHWVRQAPGKGLEWI | 67 |
| | GRIDPANGFTRYADSVKGRFTISSDNSKNTLYLQMNSLRAEDTAVYYC | |
| | AREDGYPYYTLDCWGQGTTVTVSS | |
| HFB6-3hz6-hG4P | QVQLQESGPGLVKPSGTLSLTCADSGFNIKDTYIHWVRQPPGKGLEWI | 69 |
| | GRIDPANGFTRYNPSFKSRATISSDKSKNQASLKLSSVTAADTAVYYC | |
| | AREDGYPYYTLDCWGQGTTVTVSS | |
| HFB6-3hz7-hG4P | EVQLVQSGAEVKKPGESLKISCKDSGFNIKDTYIHWVRQMPGKGLEWI | 71 |
| | GRIDPANGFTRYSPSFQGQATISADKSISTAYLQLSSLKASDTAMYYC | |
| | AREDGYPYYTLDCWGQGTTVTVSS | |
| HFB6-3hz8-hG4P | QVQLVQSGAEVKKPGASVKVSCKDSGFNIKDTYIHWVRQAPGQGLEW | 73 |
| | IGRIDPANGFTRYAQKFQGRVTMTSDTSTSTVYMELSSLRSEDTAVY | |
| | YCAREDGYPYYTLDCWGQGTTVTVSS | |
| HFB6-3hz9-hG4P | QVQLVQSGAEVKKPGASVKVSCKDSGFNIKDTYIHWVRQAPGQGLEW | 75 |
| | IGRIDPANGFTRYAQKFQGRATITSDTSTSTVYLELSSLRSEDTAVY | |
| | YCAREDGYPYYTLDCWGQGTTVTVSS | |
| HFB6-3hz10-hG4P | QVQLVQSGAEVKKPGSSVKVSCKDSGFNIKDTYIHWVRQAPGQGLEW | 77 |
| | IGRIDPANGFARYAQKFQGRATITSDESTSTAYLELSSLRSEDTAVYYC | |
| | AREDGYPYYTLDCWGQGTTVTVSS | |
| HFB6-3hz11-hG4P | QVQLVQSGAEVKKPGSSVKVSCKDSGFNIKDTYIHWVRQAPGQGLEW | 79 |
| | IGRIDPANGFARYAQKFQGRATITSDKSTSTAYLELSSLRSEDTAVYYC | |
| | AREDGYPYYTLDCWGQGTTVTVSS | |
| HFB6-3hz12-hG4P | EVQLVESGGGLVQPGGSLRLSCADSGFNIKDTYIHWVRQAPGKGLEWI | 81 |
| | GRIDPANGFTRYADSVKGRFTISSDNSKNTLYLQMNSLRAEDTAVYYC | |
| | AREDGYPYYTLDCWGQGTTVTVSS | |
| HFB6-3hz13-hG4P | QVQLQESGPGLVKPSGTLSLTCADSGFNIKDTYIHWVRQPPGKGLEWI | 83 |
| | GRIDPANGFTRYNPSFKSRATISSDKSKNQASLKLSSVTAADTAVYYCA | |
| | REDGYPYYTLDCWGQGTTVTVSS | |
| HFB6-3hz14-hG4P | EVQLVQSGAEVKKPGESLKISCKDSGFNIKDTYIHWVRQMPGKGLEWI | 85 |
| | GRIDPANGFTRYSPSFQGQATISADKSISTAYLQLSSLKASDTAMYYCA | |
| | REDGYPYYTLDCWGQGTTVTVSS | |
| HFB6-3hz15-hG4P | QVQLVQSGAEVKKPGASVKVSCKDSGFNIKDTYIHWVRQAPGQGLEW | 87 |
| | IGRIDPANGFTRYAQKFQGRVTMTSDTSTSTVYMELSSLRSEDTAVY | |
| | YCAREDGYPYYTLDCWGQGTTVTVSS | |
| HFB6-3hz16-hG4P | QVQLVQSGAEVKKPGASVKVSCKDSGFNIKDTYIHWVRQAPGQGLEW | 89 |
| | IGRIDPANGFTRYAQKFQGRATITSDTSTSTVYLELSSLRSEDTAVYY | |
| | CAREDGYPYYTLDCWGQGTTVTVSS | |
| HFB6-3hz17-hG4P | QVQLVQSGAEVKKPGSSVKVSCKDSGFNIKDTYIHWVRQAPGQGLEWI | 91 |
| | GRIDPANGFARYAQKFQGRATITSDESTSTAYLELSSLRSEDTAVYYC | |
| | AREDGYPYYTLDCWGQGTTVTVSS | |
| HFB6-3hz18-hG4P | QVQLVQSGAEVKKPGSSVKVSCKDSGFNIKDTYIHWVRQAPGQGLEWI | 93 |
| | GRIDPANGFARYAQKFQGRATITSDKSTSTAYLELSSLRSEDTAVYYC | |
| | AREDGYPYYTLDCWGQGTTVTVSS | |
| HFB6-3hz19-hG4P | EVQLVESGGGLVQPGGSLRLSCADSGFNIKDTYIHWVRQAPGKGLEWI | 95 |
| | GRIDPANGFTRYADSVKGRFTISSDNSKNTLYLQMNSLRAEDTAVYYC | |
| | AREDGYPYYTLDCWGQGTTVTVSS | |
| HFB6-3hz20-hG4P | QVQLQESGPGLVKPSGTLSLTCADSGFNIKDTYIHWVRQPPGKGLEWI | 97 |
| | GRIDPANGFTRYNPSFKSRATISSDKSKNQASLKLSSVTAADTAVYYC | |
| | AREDGYPYYTLDCWGQGTTVTVSS | |
| HFB6-3hz21-hG4P | EVQLVQSGAEVKKPGESLKISCKDSGFNIKDTYIHWVRQMPGKGLEWI | 99 |
| | GRIDPANGFTRYSPSFQGQATISADKSISTAYLQLSSLKASDTAMYYC | |
| | AREDGYPYYTLDCWGQGTTVTVSS | |
| HFB6-3hz6-hG4P- | QVQLQESGPGLVKPSGTLSLTCADSGFNIKDTYIHWVRQPPGKGLEWI | 101 |
| C109A | GRIDPANGFTRYNPSFKSRATISSDKSKNQASLKLSSVTAADTAVYYC | |
| | AREDGYPYYTLDAWGQGTTVTVSS | |
| HFB6-3hz6-hG4P- | QVQLQESGPGLVKPSGTLSLTCADSGFNIKDTYIHWVRQPPGKGLEWI | 103 |
| C109S | GRIDPANGFTRYNPSFKSRATISSDKSKNQASLKLSSVTAADTAVYYC | |
| | AREDGYPYYTLDSWGQGTTVTVSS | |
| HFB6-3hz6-hG4P- | QVQLQESGPGLVKPSGTLSLTCADSGFNIKDTYIHWVRQPPGKGLEWI | 105 |
| C109T | GRIDPANGFTRYNPSFKSRATISSDKSKNQASLKLSSVTAADTAVYYC | |
| | AREDGYPYYTLDTWGQGTTVTVSS | |
| HFB6-3hz6-hG4P- | QVQLQESGPGLVKPSGTLSLTCADSGFNIKDTYIHWVRQPPGKGLEWI | 107 |
| C109V | GRIDPANGFTRYNPSFKSRATISSDKSKNQASLKLSSVTAADTAVYYC | |
| | AREDGYPYYTLDVWGQGTTVTVSS | |
| HFB6-3hz13-hG4P | QVQLQESGPGLVKPSGTLSLTCADSGFNIKDTYIHWVRQPPGKGLEWI | 109 |
| -C109A | GRIDPANGFTRYNPSFKSRATISSDKSKNQASLKLSSVTAADTAVYYC | |
| | AREDGYPYYTLDAWGQGTTVTVSS | |
| HFB6-3hz13-hG4P | QVQLQESGPGLVKPSGTLSLTCADSGFNIKDTYIHWVRQPPGKGLEWI | 111 |
| -C109S | GRIDPANGFTRYNPSFKSRATISSDKSKNQASLKLSSVTAADTAVYYC | |
| | AREDGYPYYTLDSWGQGTTVTVSS | |
| HFB6-3hz13-hG4P | QVQLQESGPGLVKPSGTLSLTCADSGFNIKDTYIHWVRQPPGKGLEWI | 113 |
| -C109T | GRIDPANGFTRYNPSFKSRATISSDKSKNQASLKLSSVTAADTAVYYC | |
| | AREDGYPYYTLDTWGQGTTVTVSS | |
| HFB6-3hz13-hG4P | QVQLQESGPGLVKPSGTLSLTCADSGFNIKDTYIHWVRQPPGKGLEWI | 115 |
| -C109V | GRIDPANGFTRYNPSFKSRATISSDKSKNQASLKLSSVTAADTAVYYC | |
| | AREDGYPYYTLDVWGQGTTVTVSS | |
| HFB6-3hz20-hG4P | QVQLQESGPGLVKPSGTLSLTCADSGFNIKDTYIHWVRQPPGKGLEWI | 117 |
| -C109A | GRIDPANGFTRYNPSFKSRATISSDKSKNQASLKLSSVTAADTAVYYCA | |
| | REDGYPYYTLDAWGQGTTVTVSS | |
| HFB6-3hz20-hG4P | QVQLQESGPGLVKPSGTLSLTCADSGFNIKDTYIHWVRQPPGKGLEWI | 119 |
| -C109S | GRIDPANGFTRYNPSFKSRATISSDKSKNQASLKLSSVTAADTAVYYCA | |
| | REDGYPYYTLDSWGQGTTVTVSS | |
| HFB6-3hz20-hG4P | QVQLQESGPGLVKPSGTLSLTCADSGFNIKDTYIHWVRQPPGKGLEWI | 121 |
| -C109T | GRIDPANGFTRYNPSFKSRATISSDKSKNQASLKLSSVTAADTAVYYCA | |
| | REDGYPYYTLDTWGQGTTVTVSS | |
| HFB6-3hz20-hG4P | QVQLQESGPGLVKPSGTLSLTCADSGFNIKDTYIHWVRQPPGKGLEWI | 123 |
| -C109V | GRIDPANGFTRYNPSFKSRATISSDKSKNQASLKLSSVTAADTAVYYCA | |
| | REDGYPYYTLDVWGQGTTVTVSS | |
| HFB6-3hz22-hG4P | EVQLVESGGGLVQPGGSLRLSCADSGFTIKDTYIHWVRQAPGKGLEW | 125 |
| -C109A | VGRIDPANGFTRYADSVKGRFTISSDNSKNTLYLQMNSLRAEDTAVYY | |
| | CAREDGYPYYTLDAWGQGTTVTVSS | |
| HFB6-3hz23-hG4P | EVQLVESGGGLVQPGGSLRLSCAASGFTIKDTYIHWVRQAPGKGLEW | 127 |
| -C109A | VGRIDSANGFTRYADSVKGRFTISSDNSKNTLYLQMNSLRAEDTAVYY | |
| | CAREDGYPYYTLDAWGQGTTVTVSS | |
| HFB6-3hz24-hG4P | QVQLQESGPGLVKPSGTLSLTCADSGFNIKDTYIHWVRQPPGKGLEWI | 129 |
| -C109A | GRIDPANGFTRYNPSLKSRVTISSDKSKNQASLKLSSVTAADTAVYYC | |
| | AREDGYPYYTLDAWGQGTTVTVSS | |
| HFB6-3hz25-hG4P | QVQLQESGPGLVKPSGTLSLTCADSGFSISDTYIHWVRQPPGKGLEWIG | 131 |
| -C109A | RIDPANGFTRYNPSLKSRVTISSDKSKNQASLKLSSVTAADTAVYYCAR | |
| | EDGYPYYTLDAWGQGTTVTVSS | |
| HFB6-3hz26-hG4P | QVQLQESGPGLVKPSGTLSLTCAVSGFSISDTYIHWVRQPPGKGLEWI | 133 |
| -C109A | GRIDPANGFTRYNPSLKSRVTISVDKSKNQASLKLSSVTAADTAVYYC | |
| | AREDGYPYYTLDAWGQGTTVTVSS | |
| HFB6-3hz27-hG4P | QVQLQESGPGLVKPSGTLSLTCAVSGFSISDTYIHWVRQPPGKGLEWIG | 135 |
| -C109A | RIDSANGFTRYNPSLKSRVTISVDKSKNQASLKLSSVTAADTAVYYCA | |
| | REDGYPYYTLDAWGQGTTVTVSS | |
| HFB6-3hz22-hG4P | EVQLVESGGGLVQPGGSLRLSCADSGFTIKDTYIHWVRQAPGKGLEW | 137 |
| -C109S | VGRIDPANGFTRYADSVKGRFTISSDNSKNTLYLQMNSLRAEDTAVYY | |
| | CAREDGYPYYTLDSWGQGTTVTVSS | |
| HFB6-3hz23-hG4P | EVQLVESGGGLVQPGGSLRLSCAASGFTIKDTYIHWVRQAPGKGLEW | 139 |
| -C109S | VGRIDSANGFTRYADSVKGRFTISSDNSKNTLYLQMNSLRAEDTAVYY | |
| | CAREDGYPYYTLDSWGQGTTVTVSS | |
| HFB6-3hz24-hG4P | QVQLQESGPGLVKPSGTLSLTCADSGFNIKDTYIHWVRQPPGKGLEWI | 141 |
| -C109S | GRIDPANGFTRYNPSLKSRVTISSDKSKNQASLKLSSVTAADTAVYYC | |
| | AREDGYPYYTLDSWGQGTTVTVSS | |
| HFB6-3hz25-hG4P | QVQLQESGPGLVKPSGTLSLTCADSGFSISDTYIHWVRQPPGKGLEWIG | 143 |
| -C109S | RIDPANGFTRYNPSLKSRVTISSDKSKNQASLKLSSVTAADTAVYYCAR | |
| | EDGYPYYTLDSWGQGTTVTVSS | |
| HFB6-3hz26-hG4P | QVQLQESGPGLVKPSGTLSLTCAVSGFSISDTYIHWVRQPPGKGLEWIG | 145 |
| -C109S | RIDPANGFTRYNPSLKSRVTISVDKSKNQASLKLSSVTAADTAVYYCA | |
| | REDGYPYYTLDSWGQGTTVTVSS | |
| HFB6-3hz27- | QVQLQESGPGLVKPSGTLSLTCAVSGFSISDTYIHWVRQPPGKGLEWIG | 147 |
| hG4P-C109S | RIDSANGFTRYNPSLKSRVTISVDKSKNQASLKLSSVTAADTAVYYCA | |
| | REDGYPYYTLDSWGQGTTVTVSS | |

TABLE 10
Sequences of humanized light chain variable regions for HFB6-2 and
HFB6-3 mAbs.

| | | SEQ |
|---|---|---|
| | | ID |
| VL | VL sequence | NO: |
| HFB6-2hz1-hG4P | DVVMTQSPLSLPVTLGQPASISCRSSQSLLDSDGKPYLNWLQQRPGQSP | 18 |
| | RRLIYLVSKRDSGVPDRFSGSGSGTDFTLKISRVEAEGVGVYYCWQDT | |
| | HFPRTFGQGTKLEIK | |
| HFB6-2hz2-hG4P | DVVMTQSPLSLPVTLGQPASISCRSSQSLLDSDGKPYLNWLQQRPGQSP | 20 |
| | RRLIYLVSKRDSGVPDRFSGSGSGTDFTLKISRVEAEGVGVYYCWQDT | |
| | HFPRTFGQGTKLEIK | |
| HFB6-2hz3-hG4P | DVVMTQSPLSLPVTLGQPASISCRSSQSLLDSDGKPYLNWLQQRPGQSP | 22 |
| | RRLIYLVSKRDSGVPDRFSGSGSGTDFTLKISRVEAEGVGVYYCWQDT | |
| | HFPRTFGQGTKLEIK | |
| HFB6-2hz4-hG4P | DVVMTQSPLSLPVTLGQPASISCRSSQSLLDSDGKPYLNWLQQRPGQSP | 24 |
| | RRLIYLVSKRDSGVPDRFSGSGSGTDFTLKISRVEAEGVGVYYCWQDT | |
| | HFPRTFGQGTKLEIK | |
| HFB6-2hz5-hG4P | DVVMTQSPLSLPVTLGQPASISCRSSQSLLDSDGKPYLNWLQQRPGQSP | 26 |
| | RRLIYLVSKRDSGVPDRFSGSGSGTDFTLKISRVEAEGVGVYYCWQDT | |
| | HFPRTFGQGTKLEIK | |
| HFB6-2hz6-hG4P | DVVMTQSPLSLPVTLGQPASISCRSSQSLLDSDGKPYLNWLQQRPGQSP | 28 |
| | RRLIYLVSKRDSGVPDRFSGSGSGTDFTLKISRVEAEGVGVYYCWQDT | |
| | HFPRTFGQGTKLEIK | |
| HFB6-2hz7-hG4P | DVVMTQSPLSLPVTLGQPASISCRSSQSLLDSDGKPYLNWLQQRPGQSP | 30 |
| | RRLIYLVSKRDSGVPDRFSGSGSGTDFTLKISRVEAEGVGVYYCWQDT | |
| | HFPRTFGQGTKLEIK | |
| HFB6-2hz8-hG4P | EIVLTQSPGTLSLSPGERATLSCRSSQSLLDSDGKPYLNWLQQKPGQAP | 32 |
| | RRLIYLVSKLDSGIPDRFSGSGSGTDFTLTISRLEPEDLAVYYCWQDTHF | |
| | PRTFGQGTKLEIK | |
| HFB6-2hz9-hG4P | EIVLTQSPGTLSLSPGERATLSCRSSQSLLDSDGKPYLNWLQQKPGQAP | 34 |
| | RRLIYLVSKLDSGIPDRFSGSGSGTDFTLTISRLEPEDLAVYYCWQDTHF | |
| | PRTFGQGTKLEIK | |
| HFB6-2hz10-hG4P | EIVLTQSPGTLSLSPGERATLSCRSSQSLLDSDGKPYLNWLQQKPGQAP | 36 |
| | RRLIYLVSKLDSGIPDRFSGSGSGTDFTLTISRLEPEDLAVYYCWQDTHF | |
| | PRTFGQGTKLEIK | |
| HFB6-2hz11-hG4P | EIVLTQSPGTLSLSPGERATLSCRSSQSLLDSDGKPYLNWLQQKPGQAP | 38 |
| | RRLIYLVSKLDSGIPDRFSGSGSGTDFTLTISRLEPEDLAVYYCWQDTHF | |
| | PRTFGQGTKLEIK | |
| HFB6-2hz12-hG4P | EIVLTQSPGTLSLSPGERATLSCRSSQSLLDSDGKPYLNWLQQKPGQAP | 40 |
| | RRLIYLVSKLDSGIPDRFSGSGSGTDFTLTISRLEPEDLAVYYCWQDTHF | |
| | PRTFGQGTKLEIK | |
| HFB6-2hz13-hG4P | EIVLTQSPGTLSLSPGERATLSCRSSQSLLDSDGKPYLNWLQQKPGQAP | 42 |
| | RRLIYLVSKLDSGIPDRFSGSGSGTDFTLTISRLEPEDLAVYYCWQDTHF | |
| | PRTFGQGTKLEIK | |
| HFB6-2hz14-hG4P | EIVLTQSPGTLSLSPGERATLSCRSSQSLLDSDGKPYLNWLQQKPGQAP | 44 |
| | RRLIYLVSKLDSGIPDRFSGSGSGTDFTLTISRLEPEDLAVYYCWQDTHF | |
| | PRTFGQGTKLEIK | |
| HFB6-2hz15-hG4P | DIVMTQSPDSLAVSLGERATINCKSSQSLLDSDGKPYLNWLQQKPGQS | 46 |
| | PKRLIYLVSKLDSGVPDRFSGSGSGTDFTLTISSVQAEDVAVYYCWQD | |
| | THFPRTFGQGTKLEIK | |
| HFB6-2hz16-hG4P | DIVMTQSPDSLAVSLGERATINCKSSQSLLDSDGKPYLNWLQQKPGQS | 48 |
| | PKRLIYLVSKLDSGVPDRFSGSGSGTDFTLTISSVQAEDVAVYYCWQD | |
| | THFPRTFGQGTKLEIK | |
| HFB6-2hz17-hG4P | DIVMTQSPDSLAVSLGERATINCKSSQSLLDSDGKPYLNWLQQKPGQS | 50 |
| | PKRLIYLVSKLDSGVPDRFSGSGSGTDFTLTISSVQAEDVAVYYCWQD | |
| | THFPRTFGQGTKLEIK | |
| HFB6-2hz18-hG4P | DIVMTQSPDSLAVSLGERATINCKSSQSLLDSDGKPYLNWLQQKPGQS | 52 |
| | PKRLIYLVSKLDSGVPDRFSGSGSGTDFTLTISSVQAEDVAVYYCWQD | |
| | THFPRTFGQGTKLEIK | |
| HFB6-2hz19-hG4P | DIVMTQSPDSLAVSLGERATINCKSSQSLLDSDGKPYLNWLQQKPGQS | 54 |
| | PKRLIYLVSKLDSGVPDRFSGSGSGTDFTLTISSVQAEDVAVYYCWQD | |
| | THFPRTFGQGTKLEIK | |
| HFB6-2hz20-hG4P | DIVMTQSPDSLAVSLGERATINCKSSQSLLDSDGKPYLNWLQQKPGQS | 56 |
| | PKRLIYLVSKLDSGVPDRFSGSGSGTDFTLTISSVQAEDVAVYYCWQD | |
| | THFPRTFGQGTKLEIK | |
| HFB6-2hz21-hG4P | DIVMTQSPDSLAVSLGERATINCKSSQSLLDSDGKPYLNWLQQKPGQS | 58 |
| | PKRLIYLVSKLDSGVPDRFSGSGSGTDFTLTISSVQAEDVAVYYCWQD | |
| | THFPRTFGQGTKLEIK | |
| HFB6-3hz1-hG4P | DVVMTQSPLSLPLTLGQPASISCRSSQSLLDVDGKTYLNWFQQRPGQSP | 60 |
| | RRLIYLVSKLDSGVPDRFSGSGSGTDFTLKISRVEAEDVGVYYCWQGT | |
| | HFPRTFGQGTKLEIK | |
| HFB6-3hz2-hG4P | DVVMTQSPLSLPLTLGQPASISCRSSQSLLDVDGKTYLNWFQQRPGQSP | 62 |
| | RRLIYLVSKLDSGVPDRFSGSGSGTDFTLKISRVEAEDVGVYYCWQGT | |
| | HFPRTFGQGTKLEIK | |
| HFB6-3hz3-hG4P | DVVMTQSPLSLPLTLGQPASISCRSSQSLLDVDGKTYLNWFQQRPGQSP | 64 |
| | RRLIYLVSKLDSGVPDRFSGSGSGTDFTLKISRVEAEDVGVYYCWQGT | |
| | HFPRTFGQGTKLEIK | |
| HFB6-3hz4-hG4P | DVVMTQSPLSLPLTLGQPASISCRSSQSLLDVDGKTYLNWFQQRPGQSP | 66 |
| | RRLIYLVSKLDSGVPDRFSGSGSGTDFTLKISRVEAEDVGVYYCWQGT | |
| | HFPRTFGQGTKLEIK | |
| HFB6-3hz5-hG4P | DVVMTQSPLSLPLTLGQPASISCRSSQSLLDVDGKTYLNWFQQRPGQSP | 68 |
| | RRLIYLVSKLDSGVPDRFSGSGSGTDFTLKISRVEAEDVGVYYCWQGT | |
| | HFPRTFGQGTKLEIK | |
| HFB6-3hz6-hG4P | DVVMTQSPLSLPLTLGQPASISCRSSQSLLDVDGKTYLNWFQQRPGQSP | 70 |
| | RRLIYLVSKLDSGVPDRFSGSGSGTDFTLKISRVEAEDVGVYYCWQGT | |
| | HFPRTFGQGTKLEIK | |
| HFB6-3hz7-hG4P | DVVMTQSPLSLPLTLGQPASISCRSSQSLLDVDGKTYLNWFQQRPGQSP | 72 |
| | RRLIYLVSKLDSGVPDRFSGSGSGTDFTLKISRVEAEDVGVYYCWQGT | |
| | HFPRTFGQGTKLEIK | |
| HFB6-3hz8-hG4P | EIVMTQSPATLSLSPGERATISCRSSQSLLDVDGKTYLNWFQQKPGQAP | 74 |
| | RRLIYLVSKRDSGVPARFSGSGSGTDFTLTISSVQPEDFAVYYCWQGTH | |
| | FPRTFGQGTKLEIK | |
| HFB6-3hz9-hG4P | EIVMTQSPATLSLSPGERATISCRSSQSLLDVDGKTYLNWFQQKPGQAP | 76 |
| | RRLIYLVSKRDSGVPARFSGSGSGTDFTLTISSVQPEDFAVYYCWQGTH | |
| | FPRTFGQGTKLEIK | |
| HFB6-3hz10-hG4P | EIVMTQSPATLSLSPGERATISCRSSQSLLDVDGKTYLNWFQQKPGQAP | 78 |
| | RRLIYLVSKRDSGVPARFSGSGSGTDFTLTISSVQPEDFAVYYCWQGTH | |
| | FPRTFGQGTKLEIK | |
| HFB6-3hz11-hG4P | EIVMTQSPATLSLSPGERATISCRSSQSLLDVDGKTYLNWFQQKPGQAP | 80 |
| | RRLIYLVSKRDSGVPARFSGSGSGTDFTLTISSVQPEDFAVYYCWQGTH | |
| | FPRTFGQGTKLEIK | |
| HFB6-3hz12-hG4P | EIVMTQSPATLSLSPGERATISCRSSQSLLDVDGKTYLNWFQQKPGQAP | 82 |
| | RRLIYLVSKRDSGVPARFSGSGSGTDFTLTISSVQPEDFAVYYCWQGTH | |
| | FPRTFGQGTKLEIK | |
| HFB6-3hz13-hG4P | EIVMTQSPATLSLSPGERATISCRSSQSLLDVDGKTYLNWFQQKPGQAP | 84 |
| | RRLIYLVSKRDSGVPARFSGSGSGTDFTLTISSVQPEDFAVYYCWQGTH | |
| | FPRTFGQGTKLEIK | |
| HFB6-3hz14-hG4P | EIVMTQSPATLSLSPGERATISCRSSQSLLDVDGKTYLNWFQQKPGQAP | 86 |
| | RRLIYLVSKRDSGVPARFSGSGSGTDFTLTISSVQPEDFAVYYCWQGTH | |
| | FPRTFGQGTKLEIK | |
| HFB6-3hz15-hG4P | DIVMTQSPDSLALSLGERATINCKSSQSLLDVDGKTYLNWFQQKPGQP | 88 |
| | PKRLIYLVSKLDSGVPDRFSGSGSGTDFTLTISSVQAEDVAVYYCWQG | |
| | THFPRTFGQGTKLEIK | |
| HFB6-3hz16-hG4P | DIVMTQSPDSLALSLGERATINCKSSQSLLDVDGKTYLNWFQQKPGQP | 90 |
| | PKRLIYLVSKLDSGVPDRFSGSGSGTDFTLTISSVQAEDVAVYYCWQG | |
| | THFPRTFGQGTKLEIK | |
| HFB6-3hz17-hG4P | DIVMTQSPDSLALSLGERATINCKSSQSLLDVDGKTYLNWFQQKPGQP | 92 |
| | PKRLIYLVSKLDSGVPDRFSGSGSGTDFTLTISSVQAEDVAVYYCWQG | |
| | THFPRTFGQGTKLEIK | |
| HFB6-3hz18-hG4P | DIVMTQSPDSLALSLGERATINCKSSQSLLDVDGKTYLNWFQQKPGQP | 94 |
| | PKRLIYLVSKLDSGVPDRFSGSGSGTDFTLTISSVQAEDVAVYYCWQG | |
| | THFPRTFGQGTKLEIK | |
| HFB6-3hz19-hG4P | DIVMTQSPDSLALSLGERATINCKSSQSLLDVDGKTYLNWFQQKPGQP | 96 |
| | PKRLIYLVSKLDSGVPDRFSGSGSGTDFTLTISSVQAEDVAVYYCWQG | |
| | THFPRTFGQGTKLEIK | |
| HFB6-3hz20-hG4P | DIVMTQSPDSLALSLGERATINCKSSQSLLDVDGKTYLNWFQQKPGQP | 98 |
| | PKRLIYLVSKLDSGVPDRFSGSGSGTDFTLTISSVQAEDVAVYYCWQG | |
| | THFPRTFGQGTKLEIK | |
| HFB6-3hz21-hG4P | DIVMTQSPDSLALSLGERATINCKSSQSLLDVDGKTYLNWFQQKPGQP | 100 |
| | PKRLIYLVSKLDSGVPDRFSGSGSGTDFTLTISSVQAEDVAVYYCWQG | |
| | THFPRTFGQGTKLEIK | |
| HFB6-3hz6-hG4P- | DVVMTQSPLSLPLTLGQPASISCRSSQSLLDVDGKTYLNWFQQRPGQSP | 102 |
| C109A | RRLIYLVSKLDSGVPDRFSGSGSGTDFTLKISRVEAEDVGVYYCWQGT | |
| | HFPRTFGQGTKLEIK | |
| HFB6-3hz6-hG4P- | DVVMTQSPLSLPLTLGQPASISCRSSQSLLDVDGKTYLNWFQQRPGQSP | 104 |
| C109S | RRLIYLVSKLDSGVPDRFSGSGSGTDFTLKISRVEAEDVGVYYCWQGT | |
| | HFPRTFGQGTKLEIK | |
| HFB6-3hz6-hG4P- | DVVMTQSPLSLPLTLGQPASISCRSSQSLLDVDGKTYLNWFQQRPGQSP | 106 |
| C109T | RRLIYLVSKLDSGVPDRFSGSGSGTDFTLKISRVEAEDVGVYYCWQGT | |
| | HFPRTFGQGTKLEIK | |
| HFB6-3hz6-hG4P- | DVVMTQSPLSLPLTLGQPASISCRSSQSLLDVDGKTYLNWFQQRPGQSP | 108 |
| C109V | RRLIYLVSKLDSGVPDRFSGSGSGTDFTLKISRVEAEDVGVYYCWQGT | |
| | HFPRTFGQGTKLEIK | |
| HFB6-3hz13-hG4P | EIVMTQSPATLSLSPGERATISCRSSQSLLDVDGKTYLNWFQQKPGQAP | 110 |
| -C109A | RRLIYLVSKRDSGVPARFSGSGSGTDFTLTISSVQPEDFAVYYCWQGTH | |
| | FPRTFGQGTKLEIK | |
| HFB6-3hz13-hG4P | EIVMTQSPATLSLSPGERATISCRSSQSLLDVDGKTYLNWFQQKPGQAP | 112 |
| -C109S | RRLIYLVSKRDSGVPARFSGSGSGTDFTLTISSVQPEDFAVYYCWQGTH | |
| | FPRTFGQGTKLEIK | |
| HFB6-3hz13-hG4P | EIVMTQSPATLSLSPGERATISCRSSQSLLDVDGKTYLNWFQQKPGQAP | 114 |
| -C109T | RRLIYLVSKRDSGVPARFSGSGSGTDFTLTISSVQPEDFAVYYCWQGTH | |
| | FPRTFGQGTKLEIK | |
| HFB6-3hz13-hG4P | EIVMTQSPATLSLSPGERATISCRSSQSLLDVDGKTYLNWFQQKPGQAP | 116 |
| -C109V | RRLIYLVSKRDSGVPARFSGSGSGTDFTLTISSVQPEDFAVYYCWQGTH | |
| | FPRTFGQGTKLEIK | |
| HFB6-3hz20-hG4P | DIVMTQSPDSLALSLGERATINCKSSQSLLDVDGKTYLNWFQQKPGQP | 118 |
| -C109A | PKRLIYLVSKLDSGVPDRFSGSGSGTDFTLTISSVQAEDVAVYYCWQG | |
| | THFPRTFGQGTKLEIK | |
| HFB6-3hz20-hG4P | DIVMTQSPDSLALSLGERATINCKSSQSLLDVDGKTYLNWFQQKPGQP | 120 |
| -C109S | PKRLIYLVSKLDSGVPDRFSGSGSGTDFTLTISSVQAEDVAVYYCWQG | |
| | THFPRTFGQGTKLEIK | |
| HFB6-3hz20-hG4P | DIVMTQSPDSLALSLGERATINCKSSQSLLDVDGKTYLNWFQQKPGQP | 122 |
| -C109T | PKRLIYLVSKLDSGVPDRFSGSGSGTDFTLTISSVQAEDVAVYYCWQG | |
| | THFPRTFGQGTKLEIK | |
| HFB6-3hz20-hG4P | DIVMTQSPDSLALSLGERATINCKSSQSLLDVDGKTYLNWFQQKPGQP | 124 |
| -C109V | PKRLIYLVSKLDSGVPDRFSGSGSGTDFTLTISSVQAEDVAVYYCWQG | |
| | THFPRTFGQGTKLEIK | |
| HFB6-3hz22-hG4P | DIVMTQSPDSLALSLGERATINCKSSQSLLDVDGKTYLNWFQQKPGQP | 126 |
| -C109A | PKRLIYLVSKLDSGVPDRFSGSGSGTDFTLTISSVQAEDVAVYYCWQG | |
| | THFPRTFGQGTKLEIK | |
| HFB6-3hz23-hG4P | DIVMTQSPDSLALSLGERATINCKSSQSLLDVDGKTYLNWFQQKPGQP | 128 |
| -C109A | PKRLIYLVSKLDSGVPDRFSGSGSGTDFTLTISSVQAEDVAVYYCWQG | |
| | THFPRTFGQGTKLEIK | |
| HFB6-3hz24-hG4P | DIVMTQSPDSLALSLGERATINCKSSQSLLDVDGKTYLNWFQQKPGQP | 130 |
| -C109A | PKRLIYLVSKLDSGVPDRFSGSGSGTDFTLTISSVQAEDVAVYYCWQG | |
| | THFPRTFGQGTKLEIK | |
| HFB6-3hz25-hG4P | DIVMTQSPDSLALSLGERATINCKSSQSLLDVDGKTYLNWFQQKPGQP | 132 |
| -C109A | PKRLIYLVSKLDSGVPDRFSGSGSGTDFTLTISSVQAEDVAVYYCWQG | |
| | THFPRTFGQGTKLEIK | |
| HFB6-3hz26-hG4P | DIVMTQSPDSLALSLGERATINCKSSQSLLDVDGKTYLNWFQQKPGQP | 134 |
| -C109A | PKRLIYLVSKLDSGVPDRFSGSGSGTDFTLTISSVQAEDVAVYYCWQG | |
| | THFPRTFGQGTKLEIK | |
| HFB6-3hz27-hG4P | DIVMTQSPDSLALSLGERATINCKSSQSLLDVDGKTYLNWFQQKPGQP | 136 |
| -C109A | PKRLIYLVSKLDSGVPDRFSGSGSGTDFTLTISSVQAEDVAVYYCWQG | |
| | THFPRTFGQGTKLEIK | |
| HFB6-3hz22-hG4P | DIVMTQSPDSLALSLGERATINCKSSQSLLDVDGKTYLNWFQQKPGQP | 138 |
| -C109S | PKRLIYLVSKLDSGVPDRFSGSGSGTDFTLTISSVQAEDVAVYYCWQG | |
| | THFPRTFGQGTKLEIK | |
| HFB6-3hz23-hG4P | DIVMTQSPDSLALSLGERATINCKSSQSLLDVDGKTYLNWFQQKPGQP | 140 |
| -C109S | PKRLIYLVSKLDSGVPDRFSGSGSGTDFTLTISSVQAEDVAVYYCWQG | |
| | THFPRTFGQGTKLEIK | |
| HFB6-3hz24-hG4P | DIVMTQSPDSLALSLGERATINCKSSQSLLDVDGKTYLNWFQQKPGQP | 142 |
| -C109S | PKRLIYLVSKLDSGVPDRFSGSGSGTDFTLTISSVQAEDVAVYYCWQG | |
| | THFPRTFGQGTKLEIK | |
| HFB6-3hz25-hG4P | DIVMTQSPDSLALSLGERATINCKSSQSLLDVDGKTYLNWFQQKPGQP | 144 |
| -C109S | PKRLIYLVSKLDSGVPDRFSGSGSGTDFTLTISSVQAEDVAVYYCWQG | |
| | THFPRTFGQGTKLEIK | |
| HFB6-3hz26-hG4P | DIVMTQSPDSLALSLGERATINCKSSQSLLDVDGKTYLNWFQQKPGQP | 146 |
| -C109S | PKRLIYLVSKLDSGVPDRFSGSGSGTDFTLTISSVQAEDVAVYYCWQG | |
| | THFPRTFGQGTKLEIK | |
| HFB6-3hz27- | DIVMTQSPDSLALSLGERATINCKSSQSLLDVDGKTYLNWFQQKPGQP | 148 |
| hG4P-C109S | PKRLIYLVSKLDSGVPDRFSGSGSGTDFTLTISSVQAEDVAVYYCWQG | |
| | THFPRTFGQGTKLEIK | |

TABLE 11
CDR regions 1-3 of humanized heavy chain for humanized anti-BTLA
mAbs as determined by IMGT method.

| | | SEQ | | SEQ | | SEQ |
|---|---|---|---|---|---|---|
| | | ID | | ID | | ID |
| VH | HC CDR1 | NO: | HC CDR2 | NO: | HC CDR3 | NO: |
| HFB6-2hz1-hG4P | CTASGVNIKDTYMY | 1 | GRIDPANGYTRYD | 2 | CAVNDGYFEYFDVW | 3 |
| through | W | | | | | |
| HFB6-2hz21-hG4P | | | | | | |
| HFB6-3hz1-hG4P | CTDSGFNIKDTYIHW | 7 | GRIDPANGFTRYD | 8 | CAREDGYPYYTLDCW | 9 |
| through | | | | | | |
| HFB6-3hz21-hG4P | | | | | | |
| HFB6-3hz6-hG4P- | CTDSGFNIKDTYIHW | 7 | GRIDPANGFTRYD | 8 | CAREDGYPYYTLDAW | 149 |
| C109A | | | | | | |
| HFB6-3hz6-hG4P- | CTDSGFNIKDTYIHW | 7 | GRIDPANGFTRYD | 8 | CAREDGYPYYTLDSW | 150 |
| C109S | | | | | | |
| HFB6-3hz6-hG4P- | CTDSGFNIKDTYIHW | 7 | GRIDPANGFTRYD | 8 | CAREDGYPYYTLDTW | 151 |
| C109T | | | | | | |
| HFB6-3hz6-hG4P- | CTDSGFNIKDTYIHW | 7 | GRIDPANGFTRYD | 8 | CAREDGYPYYTLDVW | 152 |
| C109V | | | | | | |
| HFB6-3hz13- | CTDSGFNIKDTYIHW | 7 | GRIDPANGFTRYD | 8 | CAREDGYPYYTLDAW | 149 |
| hG4P-C109A | | | | | | |
| HFB6-3hz13- | CTDSGFNIKDTYIHW | 7 | GRIDPANGFTRYD | 8 | CAREDGYPYYTLDSW | 150 |
| hG4P-C109S | | | | | | |
| HFB6-3hz13- | CTDSGFNIKDTYIHW | 7 | GRIDPANGFTRYD | 8 | CAREDGYPYYTLDTW | 151 |
| hG4P-C109T | | | | | | |
| HFB6-3hz13- | CTDSGFNIKDTYIHW | 7 | GRIDPANGFTRYD | 8 | CAREDGYPYYTLDVW | 152 |
| hG4P-C109V | | | | | | |
| HFB6-3hz20- | CTDSGFNIKDTYIHW | 7 | GRIDPANGFTRYD | 8 | CAREDGYPYYTLDAW | 149 |
| hG4P-C109A | | | | | | |
| HFB6-3hz20- | CTDSGFNIKDTYIHW | 7 | GRIDPANGFTRYD | 8 | CAREDGYPYYTLDSW | 150 |
| hG4P-C109S | | | | | | |
| HFB6-3hz20- | CTDSGFNIKDTYIHW | 7 | GRIDPANGFTRYD | 8 | CAREDGYPYYTLDTW | 151 |
| hG4P-C109T | | | | | | |
| HFB6-3hz20- | CTDSGFNIKDTYIHW | 7 | GRIDPANGFTRYD | 8 | CAREDGYPYYTLDVW | 152 |
| hG4P-C109V | | | | | | |
| HFB6-3hz22- | CTDSGFNIKDTYIHW | 7 | GRIDPANGFTRYD | 8 | CAREDGYPYYTLDAW | 149 |
| hG4P-C109A | | | | | | |
| HFB6-3hz23- | CTDSGFNIKDTYIHW | 7 | GRIDPANGFTRYD | 8 | CAREDGYPYYTLDAW | 149 |
| hG4P-C109A | | | | | | |
| HFB6-3hz24- | CTDSGFNIKDTYIHW | 7 | GRIDPANGFTRYD | 8 | CAREDGYPYYTLDAW | 149 |
| hG4P-C109A | | | | | | |
| HFB6-3hz25- | CTDSGFNIKDTYIHW | 7 | GRIDPANGFTRYD | 8 | CAREDGYPYYTLDAW | 149 |
| hG4P-C109A | | | | | | |
| HFB6-3hz26- | CTDSGFNIKDTYIHW | 7 | GRIDPANGFTRYD | 8 | CAREDGYPYYTLDAW | 149 |
| hG4P-C109A | | | | | | |
| HFB6-3hz27- | CTDSGFNIKDTYIHW | 7 | GRIDPANGFTRYD | 8 | CAREDGYPYYTLDAW | 149 |
| hG4P-C109A | | | | | | |
| HFB6-3hz22- | CTDSGFNIKDTYIHW | 7 | GRIDPANGFTRYD | 8 | CAREDGYPYYTLDSW | 150 |
| hG4P-C109S | | | | | | |
| HFB6-3hz23- | CTDSGFNIKDTYIHW | 7 | GRIDPANGFTRYD | 8 | CAREDGYPYYTLDSW | 150 |
| hG4P-C109S | | | | | | |
| HFB6-3hz24- | CTDSGFNIKDTYIHW | 7 | GRIDPANGFTRYD | 8 | CAREDGYPYYTLDSW | 150 |
| hG4P-C109S | | | | | | |
| HFB6-3hz25- | CTDSGFNIKDTYIHW | 7 | GRIDPANGFTRYD | 8 | CAREDGYPYYTLDSW | 150 |
| hG4P-C109S | | | | | | |
| HFB6-3hz26- | CTDSGFNIKDTYIHW | 7 | GRIDPANGFTRYD | 8 | CAREDGYPYYTLDSW | 150 |
| hG4P-C109S | | | | | | |
| HFB6-3hz27- | CTDSGFNIKDTYIHW | 7 | GRIDPANGFTRYD | 8 | CAREDGYPYYTLDSW | 150 |
| hG4P-C109S | | | | | | |

TABLE 12
CDR regions 1-3 of humanized light
chain for humanized anti-BTLA
mAbs as determined by IMGT method.

| | | SEQ | | SEQ | | SEQ |
|---|---|---|---|---|---|---|
| | LC | ID | LC | ID | LC | ID |
| VL | CDR1 | NO: | CDR2 | NO: | CDR3 | NO: |
| HFB6-2hz1- | CKSSQ | 4 | LVSK | 5 | CWQDT | 6 |
| hG4P | SLLDS | | LDS | | HFPRT | |
| through | DGKPY | | | | F | |
| HFB6-2hz21- | LNW | | | | | |
| hG4P | | | | | | |
| HFB6-3hz1- | CKSSQ | 10 | LVSK | 11 | CWQGT | 12 |
| hG4P | SLLDV | | LDS | | HFPRT | |
| through | DGKTY | | | | F | |
| HFB6-3hz27- | LNW | | | | | |
| hG4P- | | | | | | |
| C109S | | | | | | |

### Example 9: Binding and Cross-Reactivity of HFB6-3 Humanized Variants with Cysteine Mutation

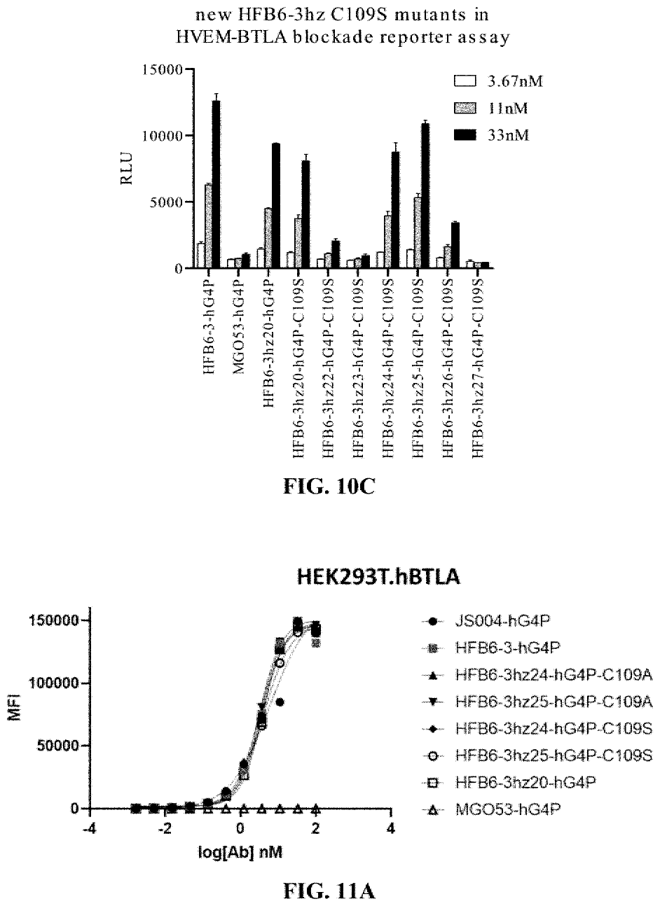

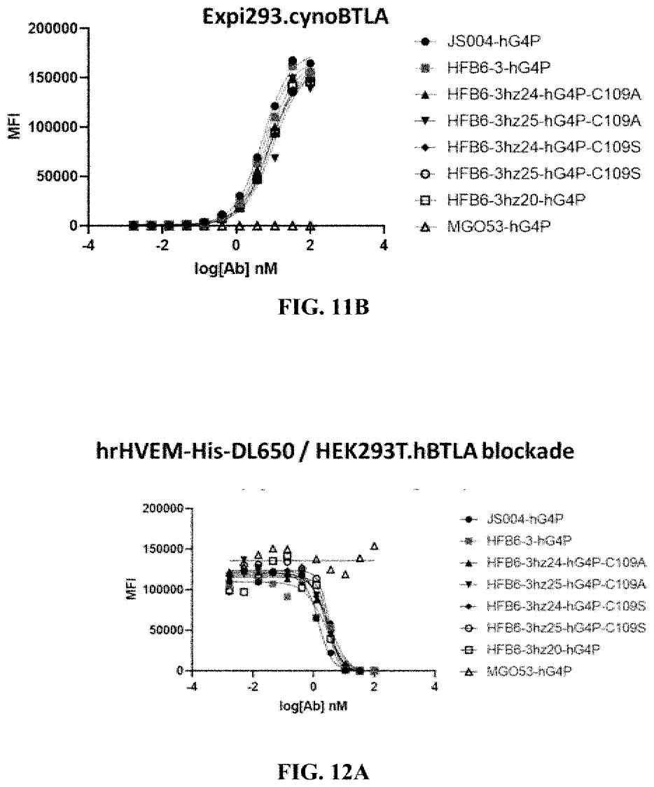

In FIGS. 11A and 11B, HEK293 overexpressing human BTLA (FIG. 11A) and Expi293 cells overexpressing cynomolgus BTLA (FIG. 11B) were incubated with different concentrations of selected HFB6-3 humanized variants with cysteine mutations. Anti-BTLA antibodies binding to target cells were quantified with an AF647-conjugated anti-human IgG Fc 2nd antibody by FACS. HFB6-3hz24-hG4P-C109A, HFB6-3hz25-hG4P-C109A, HFB6-3hz24-hG4P-C109S and HFB6-3hz25-hG4P-C109S bound to human BTLA and cyno BTLA with single digit nM and sub-nM EC₅₀, similar as parental HFB6-3 (FIGS. 11A and 11B and Table 13).

TABLE 13
HFB6-3 humanized antibodies were capable
of binding human and cyno BTLA.

| | HEK293T.hBTLA | Expi293.cynoBTLA |
|---|---|---|
| | binding EC₅₀ | binding EC₅₀ |
| Antibody | (nM) | (nM) |
| JS004-hG4P | 5.971 | 5.245 |
| HFB6-3-hG4P | 3.206 | 5.695 |
| HFB6-3hz24-hG4P-C109A | 3.265 | 6.895 |
| HFB6-3hz25-hG4P-C-109A | 3.196 | 9.686 |
| HFB6-3hz24-hG4P-C109S | 3.595 | 7.828 |
| HFB6-3hz25-hG4P-C109S | 4.037 | 8.828 |
| HFB6-3z20-hG4P | 3.844 | 7.375 |

### Example 10: Blocking Activity of HFB6-3 Humanized Variants with Cysteine Mutation

This example demonstrates that selected HFB6-3 humanized variants with cysteine mutation blocked the interaction between recombinant HVEM soluble protein with cellular BTLA and relieved HVEM-mediated TCR inhibition in T cells, comparable to parental HFB6-3 antibody.

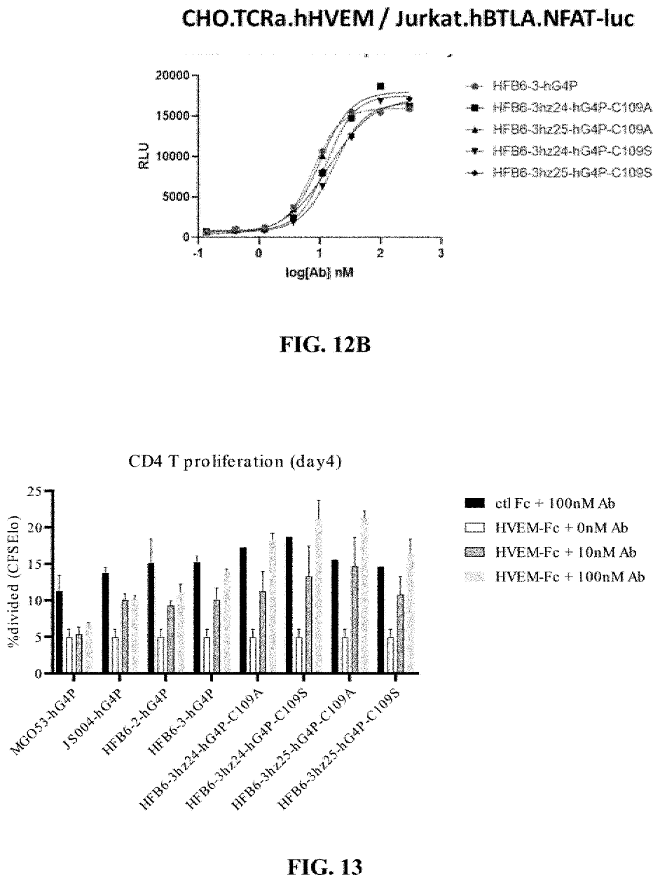

In FIG. 12A, recombinant HVEM soluble protein was conjugated with DyLight 650 (DL650) fluorophore. HEK293 cells overexpressing hBTLA (HEK293.BTLA) were preincubated with increasing concentrations of selected HFB6-3 humanized variants with cysteine mutants or parental HFB6-3 at 4° C. for 30 minutes and then with DL650-conjugated HVEM for additional 30 minutes. After washing, soluble HVEM remaining associated with BTLA on HEK293.BTLA cells was measured by FACS. HFB6-3hz24-hG4P-C109A, HFB6-3hz25-hG4P-C109A, HFB6-3hz24-hG4P-C109S and HFB6-3hz25-hG4P-C109S blocked soluble HVEM and cellular BTLA interaction with single digit nM IC₅₀, which was comparable to parental HFB6-3 (FIG. 12A and Table 14).

TABLE 14
HFB6-3 humanized antibodies blocked soluble
HVEM and cellular BTLA interaction.

| | hrHVEM-His-DL650/HEK293T.hBTLA |
|---|---|
| Antibody | blockade (IC₅₀ (nM)) |
| JS004-hG4P | 1.472 |
| HFB6-3-hG4P | 2.690 |
| HFB6-3hz24-hG4P-C109A | 2.636 |
| HFB6-3hz25-hG4P-C109A | 2.611 |
| HFB6-3hz24-hG4P-C109S | 3.216 |
| HFB6-3hz25-hG4P-C109S | 3.203 |
| HFB6-3hz20-hG4P | 2.906 |

In FIG. 12B, the functional blocking activity of selected HFB6-3 humanized variants with cysteine mutation was evaluated in a HVEM-BTLA blockade reporter assay. In this assay, a CHO cell line engineered to express a TCR activator (TCRa) and HVEM (the ligand of BTLA) was co-cultured with a Jurkat reporter cell line that expresses BTLA and a luciferase reporter driven by NFAT binding promoter. Application of HFB6-3hz24-hG4P-C109A, HFB6-3hz25-hG4P-C109A, HFB6-3hz24-hG4P-C109S and HFB6-3hz25-hG4P-C109S restored luciferase reporter signal in a dose-dependent manner similar as parental HFB6-3 (FIG. 12B and Table 15).

TABLE 15
HFB6-3 humanized antibodies blocked HVEM/BTLA interaction.

| | CHO.TCRa.hHVEM/ |
|---|---|
| | Jurkat.hBTLA.NFAT-luc |
| Antibody | (EC₅₀ (nM)) |
| HFB6-3-hG4P | 7.896 |
| HFB6-3hz24-hG4P-C109A | 13.04 |
| HFB6-3hz25-hG4P-C109A | 10.16 |
| HFB6-3hz24-hG4P-C109S | 16.60 |
| HFB6-3hz25-hG4P-C109S | 14.40 |

### Example 11: HFB6-3 Humanized Variants with Cysteine Mutation Reverse-Mediated Suppression on Primary CD4+ T-Cell Proliferation

In FIG. 13, selected HFB6-3 humanized variants with cysteine mutations were evaluated in the same assay as in example 4. HFB6-3hz24-hG4P-C109A, HFB6-3hz25-hG4P-C109A, HFB6-3hz24-hG4P-C109S and HFB6-3hz25-hG4P-C109S reversed HVEM-mediated suppression on primary CD4+ T cell proliferation similar to the parental HFB6-3 antibody.

### Example 12: HFB6-3 Humanized Variants with Cysteine Mutations Induced IFNγ And Pro-Inflammatory Cytokine Production in Primary Dissociated Tumor Culture

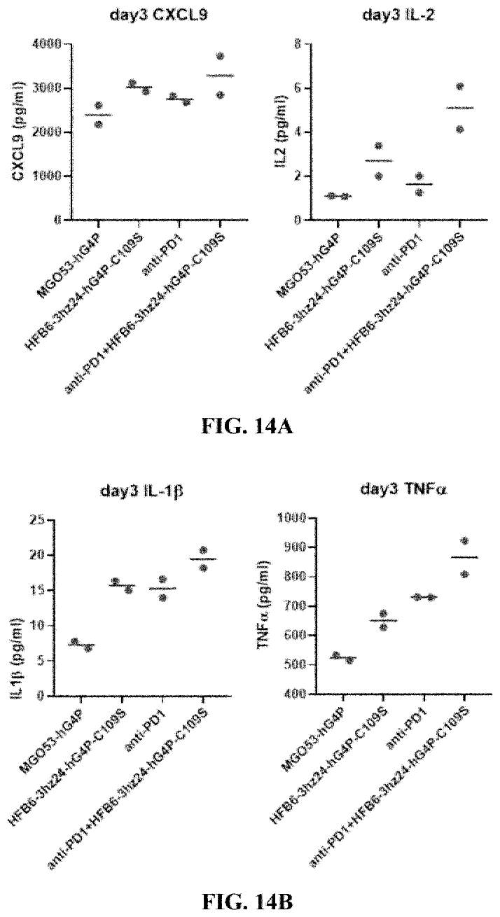

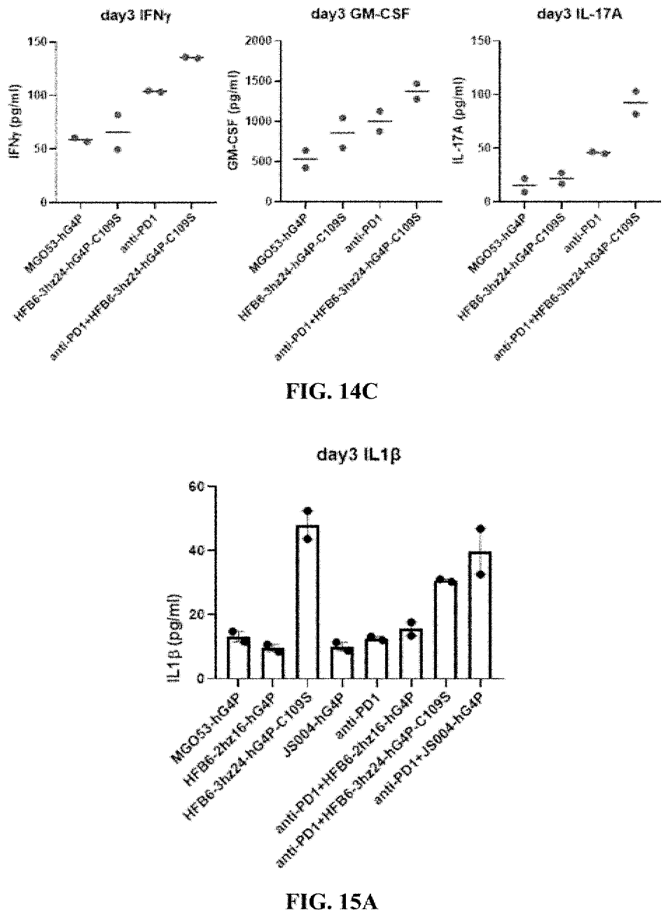

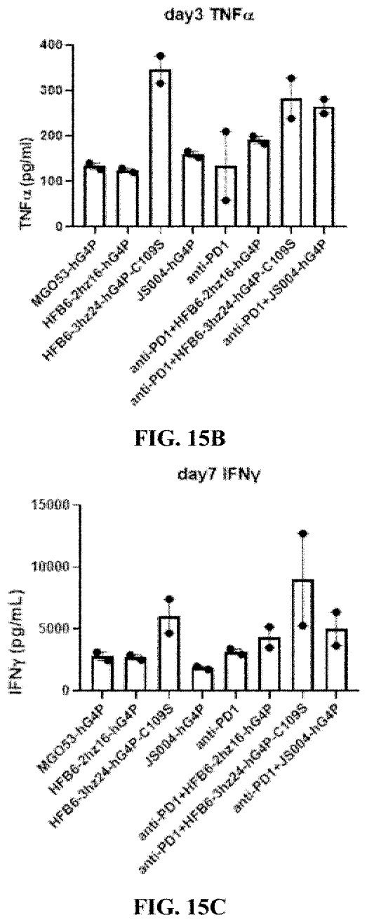

In FIGS. 14A-14C and FIGS. 15A-15C, the assay is the same as in example 6. TIL F and TIL O are dissociated tumor samples from lung cancer patients. In FIGS. 14A-14C, treatment of HFB6-3hz24-hG4P-C109S in combination with anti-PD1 enhanced CXCL9, IL2, IL1β, TNFα, IFNγ, GM-CSF and IL-17A production compared with single agent treatment alone in TIL F. In FIGS. 15A-15C, HFB6-3hz24-hG4P-C109S treatment alone enhanced IL1β, TNFα and IFNγ production to a stronger extent than anti-PD1 or benchmark JS004 in TIL O.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the scope of the present invention as defined by the present description.

1. An isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 7, 8, 153, 10, 11, and 12, respectively;
(2) SEQ ID NOs: 1, 2, 3, 4, 5, and 6, respectively;
(3) SEQ ID NOs: 7, 8, 150, 10, 11, and 12, respectively;
(4) SEQ ID NOs: 1, 2, 3, 4, 5, and 6, respectively;
(5) SEQ ID NOs: 7, 8, 9, 10, 11, and 12, respectively;
(6) SEQ ID NOs: 7, 8, 149, 10, 11, and 12, respectively;
(7) SEQ ID NOs: 7, 8, 151, 10, 11, and 12, respectively; or
(8) SEQ ID NOs: 7, 8, 152, 10, 11, and 12, respectively;
wherein the antibody or antigen-binding fragment thereof specifically binds B- and T-lymphocyte attenuator (BTLA), preferably human or cynomolgus BTLA, optionally, said monoclonal antibody or antigen-binding fragment thereof is not naturally occurring.

2. The isolated monoclonal antibody or antigen-binding fragment thereof of claim 1, comprising a heavy chain variable region having a polypeptide sequence at least 95% identical to SEQ ID NO: 15, 13, 141, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 143, 145, or 147, or a light chain variable region having a polypeptide sequence at least 96% identical to SEQ ID NO: 16, 14, 142, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130 132, 134, 136, 138, 140, 144, 146, or 148.

3. The isolated monoclonal antibody or antigen-binding fragment thereof of claim 1, comprising;
(1) a heavy chain variable region having the polypeptide sequence of SEQ ID NO:15, and a light chain variable region having the polypeptide sequence of SEQ ID NO:16;
(2) a heavy chain variable region having the polypeptide sequence of SEQ ID NO:13, and a light chain variable region having the polypeptide sequence of SEQ ID NO:14;
(3) a heavy chain variable region having the polypeptide sequence of SEQ ID NO:141, and a light chain variable region having the polypeptide sequence of SEQ ID NO:142;
(4) a heavy chain variable region having the polypeptide sequence of SEQ ID NO:17, and a light chain variable region having the polypeptide sequence of SEQ ID NO:18;
(5) a heavy chain variable region having the polypeptide sequence of SEQ ID NO:19, and a light chain variable region having the polypeptide sequence of SEQ ID NO:20;
(6) a heavy chain variable region having the polypeptide sequence of SEQ ID NO:21, and a light chain variable region having the polypeptide sequence of SEQ ID NO:22;
(7) a heavy chain variable region having the polypeptide sequence of SEQ ID NO:23, and a light chain variable region having the polypeptide sequence of SEQ ID NO:24;
(8) a heavy chain variable region having the polypeptide sequence of SEQ ID NO:25, and a light chain variable region having the polypeptide sequence of SEQ ID NO:26;
(9) a heavy chain variable region having the polypeptide sequence of SEQ ID NO:27, and a light chain variable region having the polypeptide sequence of SEQ ID NO:28;
(10) a heavy chain variable region having the polypeptide sequence of SEQ ID NO:29, and a light chain variable region having the polypeptide sequence of SEQ ID NO:30;
(11) a heavy chain variable region having the polypeptide sequence of SEQ ID NO:31, and a light chain variable region having the polypeptide sequence of SEQ ID NO:32;
(12) a heavy chain variable region having the polypeptide sequence of SEQ ID NO:33, and a light chain variable region having the polypeptide sequence of SEQ ID NO:34;
(13) a heavy chain variable region having the polypeptide sequence of SEQ ID NO:35, and a light chain variable region having the polypeptide sequence of SEQ ID NO:36;
(14) a heavy chain variable region having the polypeptide sequence of SEQ ID NO:37, and a light chain variable region having the polypeptide sequence of SEQ ID NO:38;
(15) a heavy chain variable region having the polypeptide sequence of SEQ ID NO:39, and a light chain variable region having the polypeptide sequence of SEQ ID NO:40;
(16) a heavy chain variable region having the polypeptide sequence of SEQ ID NO:41, and a light chain variable region having the polypeptide sequence of SEQ ID NO:42;
(17) a heavy chain variable region having the polypeptide sequence of SEQ ID NO:43, and a light chain variable region having the polypeptide sequence of SEQ ID NO:44;
(18) a heavy chain variable region having the polypeptide sequence of SEQ ID NO:45, and a light chain variable region having the polypeptide sequence of SEQ ID NO:46;
(19) a heavy chain variable region having the polypeptide sequence of SEQ ID NO:47, and a light chain variable region having the polypeptide sequence of SEQ ID NO:48;
(20) a heavy chain variable region having the polypeptide sequence of SEQ ID NO:49, and a light chain variable region having the polypeptide sequence of SEQ ID NO:50;
(21) a heavy chain variable region having the polypeptide sequence of SEQ ID NO:51, and a light chain variable region having the polypeptide sequence of SEQ ID NO:52;
(22) a heavy chain variable region having the polypeptide sequence of SEQ ID NO:53, and a light chain variable region having the polypeptide sequence of SEQ ID NO:54;
(23) a heavy chain variable region having the polypeptide sequence of SEQ ID NO:55, and a light chain variable region having the polypeptide sequence of SEQ ID NO:56;
(24) a heavy chain variable region having the polypeptide sequence of SEQ ID NO:57, and a light chain variable region having the polypeptide sequence of SEQ ID NO:58;
(25) a heavy chain variable region having the polypeptide sequence of SEQ ID NO:59, and a light chain variable region having the polypeptide sequence of SEQ ID NO:60;
(26) a heavy chain variable region having the polypeptide sequence of SEQ ID NO:61, and a light chain variable region having the polypeptide sequence of SEQ ID NO:62;
(27) a heavy chain variable region having the polypeptide sequence of SEQ ID NO:63, and a light chain variable region having the polypeptide sequence of SEQ ID NO:64;
(28) a heavy chain variable region having the polypeptide sequence of SEQ ID NO:65, and a light chain variable region having the polypeptide sequence of SEQ ID NO:66;
(29) a heavy chain variable region having the polypeptide sequence of SEQ ID NO:67, and a light chain variable region having the polypeptide sequence of SEQ ID NO:68;
(30) a heavy chain variable region having the polypeptide sequence of SEQ ID NO:69, and a light chain variable region having the polypeptide sequence of SEQ ID NO:70;
(31) a heavy chain variable region having the polypeptide sequence of SEQ ID NO:71, and a light chain variable region having the polypeptide sequence of SEQ ID NO:72;
(32) a heavy chain variable region having the polypeptide sequence of SEQ ID NO:73, and a light chain variable region having the polypeptide sequence of SEQ ID NO:74;
(33) a heavy chain variable region having the polypeptide sequence of SEQ ID NO:75, and a light chain variable region having the polypeptide sequence of SEQ ID NO:76;
(34) a heavy chain variable region having the polypeptide sequence of SEQ ID NO:77, and a light chain variable region having the polypeptide sequence of SEQ ID NO:78;
(35) a heavy chain variable region having the polypeptide sequence of SEQ ID NO:79, and a light chain variable region having the polypeptide sequence of SEQ ID NO:80;
(36) a heavy chain variable region having the polypeptide sequence of SEQ ID NO:81, and a light chain variable region having the polypeptide sequence of SEQ ID NO:82;
(37) a heavy chain variable region having the polypeptide sequence of SEQ ID NO:83, and a light chain variable region having the polypeptide sequence of SEQ ID NO:84;
(38) a heavy chain variable region having the polypeptide sequence of SEQ ID NO:85, and a light chain variable region having the polypeptide sequence of SEQ ID NO:86;
(39) a heavy chain variable region having the polypeptide sequence of SEQ ID NO:87, and a light chain variable region having the polypeptide sequence of SEQ ID NO:88;
(40) a heavy chain variable region having the polypeptide sequence of SEQ ID NO:89, and a light chain variable region having the polypeptide sequence of SEQ ID NO:90;
(41) a heavy chain variable region having the polypeptide sequence of SEQ ID NO:91, and a light chain variable region having the polypeptide sequence of SEQ ID NO:92;
(42) a heavy chain variable region having the polypeptide sequence of SEQ ID NO:93, and a light chain variable region having the polypeptide sequence of SEQ ID NO:94;
(43) a heavy chain variable region having the polypeptide sequence of SEQ ID NO:95, and a light chain variable region having the polypeptide sequence of SEQ ID NO:96;
(44) a heavy chain variable region having the polypeptide sequence of SEQ ID NO:97, and a light chain variable region having the polypeptide sequence of SEQ ID NO:98;
(45) a heavy chain variable region having the polypeptide sequence of SEQ ID NO:99, and a light chain variable region having the polypeptide sequence of SEQ ID NO:100;
(46) a heavy chain variable region having the polypeptide sequence of SEQ ID NO:101, and a light chain variable region having the polypeptide sequence of SEQ ID NO:102;
(47) a heavy chain variable region having the polypeptide sequence of SEQ ID NO:103, and a light chain variable region having the polypeptide sequence of SEQ ID NO:104;
(48) a heavy chain variable region having the polypeptide sequence of SEQ ID NO:105, and a light chain variable region having the polypeptide sequence of SEQ ID NO:106;
(49) a heavy chain variable region having the polypeptide sequence of SEQ ID NO:107, and a light chain variable region having the polypeptide sequence of SEQ ID NO:108;
(50) a heavy chain variable region having the polypeptide sequence of SEQ ID NO:109, and a light chain variable region having the polypeptide sequence of SEQ ID NO:110;
(51) a heavy chain variable region having the polypeptide sequence of SEQ ID NO:111, and a light chain variable region having the polypeptide sequence of SEQ ID NO:112;
(52) a heavy chain variable region having the polypeptide sequence of SEQ ID NO:113, and a light chain variable region having the polypeptide sequence of SEQ ID NO:114;
(53) a heavy chain variable region having the polypeptide sequence of SEQ ID NO:115, and a light chain variable region having the polypeptide sequence of SEQ ID NO:116;
(54) a heavy chain variable region having the polypeptide sequence of SEQ ID NO:117, and a light chain variable region having the polypeptide sequence of SEQ ID NO:118;
(55) a heavy chain variable region having the polypeptide sequence of SEQ ID NO:119, and a light chain variable region having the polypeptide sequence of SEQ ID NO:120;
(56) a heavy chain variable region having the polypeptide sequence of SEQ ID NO:121, and a light chain variable region having the polypeptide sequence of SEQ ID NO:122;
(57) a heavy chain variable region having the polypeptide sequence of SEQ ID NO:123, and a light chain variable region having the polypeptide sequence of SEQ ID NO:124;
(58) a heavy chain variable region having the polypeptide sequence of SEQ ID NO:125, and a light chain variable region having the polypeptide sequence of SEQ ID NO:126;
(59) a heavy chain variable region having the polypeptide sequence of SEQ ID NO:127, and a light chain variable region having the polypeptide sequence of SEQ ID NO:128;
(60) a heavy chain variable region having the polypeptide sequence of SEQ ID NO:129, and a light chain variable region having the polypeptide sequence of SEQ ID NO:130;
(61) a heavy chain variable region having the polypeptide sequence of SEQ ID NO:131, and a light chain variable region having the polypeptide sequence of SEQ ID NO:132;
(62) a heavy chain variable region having the polypeptide sequence of SEQ ID NO:133, and a light chain variable region having the polypeptide sequence of SEQ ID NO:134;
(63) a heavy chain variable region having the polypeptide sequence of SEQ ID NO:135, and a light chain variable region having the polypeptide sequence of SEQ ID NO:136;
(64) a heavy chain variable region having the polypeptide sequence of SEQ ID NO:137, and a light chain variable region having the polypeptide sequence of SEQ ID NO:138;
(65) a heavy chain variable region having the polypeptide sequence of SEQ ID NO:139, and a light chain variable region having the polypeptide sequence of SEQ ID NO:140;
(66) a heavy chain variable region having the polypeptide sequence of SEQ ID NO:143, and a light chain variable region having the polypeptide sequence of SEQ ID NO:144;
(67) a heavy chain variable region having the polypeptide sequence of SEQ ID NO:145, and a light chain variable region having the polypeptide sequence of SEQ ID NO:146; or
(68) a heavy chain variable region having the polypeptide sequence of SEQ ID NO:147, and a light chain variable region having the polypeptide sequence of SEQ ID NO:148.

4.-7. (canceled)

8. The isolated monoclonal antibody or antigen-binding fragment thereof of claim 1, wherein the monoclonal antibody or antigen-binding fragment thereof binds to human BTLA with a K_{D} of less than about 25 nM, 20 nM, 15 nM, 5 nM, 2 nM, 1 nM, or 0.5 nM.

9. The isolated monoclonal antibody or antigen-binding fragment thereof of claim 1, wherein the monoclonal antibody or antigen-binding fragment thereof;
(1) is an agonist of human BTLA and activates downstream signaling from BTLA upon binding to BTLA;
(2) is capable of inhibiting B cell proliferation and/or T cell and/or plasma cell activation;
(3) is capable of blocking or abolishing BTLA binding to herpes virus entry mediator (HVEM); and/or
(4) is capable of increasing pro-inflammatory cytokine production; mediating recruitment of conjugated drugs; and/or forming a bispecific antibody with another mAb or antigen-binding fragment thereof with cancer-killing effect.

10. (canceled)

11. The isolated monoclonal antibody or antigen-binding fragment thereof of claim 9, wherein the T cell is selected from the group consisting of a CD4 T-cell, a CD8 T-cell, a Th1 T-cell, a TFH T-cell, an αβ T-cell, and a γδ T-cell.

12-13. (canceled)

14. The isolated monoclonal antibody or antigen-binding fragment thereof of any one of claim 9, wherein the pro-inflammatory cytokine is selected from interferon γ (IFNγ), interleukin-2 (IL-2), C—X—C motif chemokine ligand 9 (CXCL9), C—X—C motif chemokine ligand 10 (CXCL10), interleukin 1β (IL1β), and/or tumor necrosis factor α (TNFα).

15. (canceled)

16. A bispecific antibody or antigen-binding fragment thereof comprising the monoclonal antibody or antigen-binding fragment thereof of claim 1.

17. An isolated nucleic acid encoding the monoclonal antibody or antigen-binding fragment thereof of claim 1.

18. An isolated nucleic acid encoding the bispecific antibody or antigen-binding fragment thereof of claim 16.

19. A vector comprising the isolated nucleic acid of claim 17.

20. A host cell comprising the vector of claim 19.

21. A pharmaceutical composition, comprising the isolated monoclonal antibody or antigen-binding fragment thereof of claim 1 and a pharmaceutically acceptable carrier.

22. A method of targeting BTLA on a cancer cell surface, and/or treating a cancer in a subject in need thereof, comprising administering to the subject the pharmaceutical composition of claim 21, wherein;
(a) the cancer is a solid tumor, or
(b) the cancer is selected from the group consisting of melanoma, lung cancer, renal cell carcinoma, and liver cancer,
wherein the subject comprises BTLA-expressing T cells, B cells, dendritic cells (DCs), or natural killer (NK) cells.

23. The method of claim 22, wherein the solid tumor is:
(a) a solid tumor with infiltrating T cells;
(b) a solid tumor with infiltrating T effector cells;
(c) a solid tumor with T effector cells expressing BTLA; or
(d) a solid tumor with infiltrating T effector cells expressing BTLA and BTLA ligand HVEM highly expressing in the tumor microenvironment.

24-25. (canceled)

26. The method of claim 22, wherein the pharmaceutical composition further comprises a second anti-cancer agent, wherein the second anti-cancer agent is an anti-PD1 antibody or antigen-binding fragment thereof.

27. (canceled)

28. A method of producing the monoclonal antibody or antigen-binding fragment thereof of claim 1, comprising culturing a cell comprising a nucleic acid encoding the monoclonal antibody or antigen-binding fragment thereof under conditions to produce the monoclonal antibody or antigen-binding fragment thereof and recovering the monoclonal antibody or antigen-binding fragment thereof from the cell or culture.

29. A method of producing a pharmaceutical composition comprising the monoclonal antibody or antigen-binding fragment of claim 1, comprising combining the monoclonal antibody or antigen-binding fragment thereof with a pharmaceutically acceptable carrier to obtain the pharmaceutical composition.

30. A method of determining the level of BTLA in a subject, the method comprising;
(a) obtaining a sample from the subject, wherein;
(i) the sample comprises T cells, B cells, dendritic cells (DCs), or natural killer (NK) cells; and/or
(ii) the sample is a tissue sample or a blood sample, preferably wherein the tissue sample is a cancer tissue sample;
(b) contacting the sample with an isolated monoclonal antibody or antigen-binding fragment thereof of claim 1; and
(c) determining the level of BTLA in the subject.

31. The method of claim 30, wherein the tissue sample is a cancer tissue sample.

32. (canceled)
